(19) United States
(12) Patent Application Publication
(10) Pub. No.: US 2022/0378763 A1
(43) Pub. Date: Dec. 1, 2022

(54) COMPOUND FOR TREATMENT OF ACUTE INFLAMMATION IN THE MYOCARDIUM

(71) Applicant: CALPROTECT AB, Lund (SE)

(72) Inventors: Alexandru Schiopu, Malmö (SE); Goran Marinkovic, Bor (RS)

(73) Assignee: CALPROTECT AB, Lund (SE)

(21) Appl. No.: 17/621,772

(22) PCT Filed: Jun. 29, 2020
(86) PCT No.: PCT/EP2020/068243

§ 371 Date: Dec. 22, 2021

(30) Foreign Application Priority Data
Jun. 28, 2019 (EP) 19183290.6

(51) Int. Cl. A61K 31/444; A61P 9/10; A61K 31/50; A61K 31/4436

(52) U.S. Cl. CPC A61K 31/444; A61P 9/10; A61K 31/50; A61K 31/4436

(57) ABSTRACT

The present invention relates to a compound for treatment of a disease or disorder involving inflammation in the myocardium, such as for example myocardial infarction due to myocardial ischemia, myocardial infarction due to myocardial ischemia/reperfusion, myocarditis, sepsis, sepsis-induced myocardial inflammation, and septic cardiomyopathy. The present invention further relates to treatment of a disease or disorder involving inflammation in the myocardium by administration of said compound during the acute phase of said disease or disorder.

### TECHNICAL FIELD

The present invention relates to a compound for treatment of a disease or disorder involving inflammation in the myocardium, such as for example myocardial infarction due to myocardial ischemia, myocardial infarction due to myocardial ischemia/reperfusion, myocarditis, sepsis, sepsis-induced myocardial inflammation and sepsis-induced cardiomyopathy. The present invention further relates to treatment of a disease or disorder involving inflammation in the myocardium by administration of said compound during the acute phase of said disease or disorder.

### BACKGROUND

Myocardial inflammation is a common denominator of various diseases that injure the myocardium, including but not limited to: myocardial infarction due to myocardial ischemia or myocardial ischemia/reperfusion, sepsis, and myocarditis. The role of inflammation is to clear dead or dying cardiomyocytes, infectious agents and cellular debris, and to promote myocardial repair. However, excessive and prolonged inflammatory responses are also known to promote further myocardial damage through production of reactive oxygen species, cytokines, chemokines and proteases. Further, the inflammatory damage leads to loss of myocardial structure and function, followed by complications such as heart failure, arrhythmias, and myocardial rupture. Cellular effectors of innate immunity such as neutrophils, inflammatory monocytes, macrophages, dendritic cells, and eosinophils play important roles in the acute inflammatory response in the myocardium.

Although sharing these common pathways, the initial triggering factor of myocardial damage and inflammation confers particular traits to the different pathologies. In myocardial infarction (MI), the original trigger is coronary artery occlusion (total or subtotal), leading to myocardial ischemia and necrosis. The coronary occlusion can be permanent or temporary. The temporary coronary occlusion is followed by reperfusion, either spontaneous such as in non-ST-elevation myocardial infarction (NSTEMI) or mechanical, by coronary angiography followed by percutaneous coronary interventions or coronary artery by-pass grafting, such as in ST-elevation myocardial infarction (STEMI). Coronary reperfusion leads to further myocardial injury, so called ischemia/reperfusion injury, which amplifies the damage and the subsequent inflammatory response.

The immune response to MI involves two equally important, consecutive phases: the acute or inflammatory phase and the reparatory phase. An adequate balance between the two phases is crucial for efficient myocardial repair, recovery of cardiac function and a positive patient prognosis. During the inflammatory phase, neutrophils and inflammatory Ly6C^{hi} monocytes are recruited into the ischemic myocardium.

Subsequently, inflammatory Ly6C^{hi} monocytes give rise to reparatory Ly6C^{lo} macrophages, with an important role in cardiac recovery. Upregulation of the transcription factor Nur77 and of the efferocytosis receptor MER tyrosine-kinase (MerTK) on the surface of the macrophages are crucial for the transition from inflammatory monocytes to reparatory macrophages. The reparatory macrophages are important effectors of efferocytosis (cleaning of cellular debris, necrotic, and apoptotic cells) and mediate tissue healing.

Alarmins are a group of heterogeneous molecules released from dying cells and activated leukocytes that signal tissue damage and trigger an innate immune response. S100A9 and its dimerization partner S100A8 are pro-inflammatory alarmins that are produced and stored in large amounts in neutrophils. S100A8/A9, also known as calprotectin, constitutes approximately 40% of all cytosolic proteins in neutrophils and is rapidly released extracellularly upon activation. Monocytes/macrophages, endothelial cells and platelets have also been shown to be able to produce and release S100A8/A9, albeit in much smaller amounts. Extracellular S100A8/A9 can functionally be described as a damage-associated molecular pattern (DAMP) molecule which is released by damaged tissues and induces endothelial and immune cell activation by interacting with its receptors RAGE, TLR4 and EMMPRIN, located on the cell surface. During the acute phase of MI, S100A8/A9 is rapidly released from the site of the ischemic myocardial injury and increases in the coronary and systemic circulation before the classic markers of myocardial damage myoglobin and troponin. S100A8/A9 is primarily derived from necrotic cardiomyocytes, neutrophils and monocytes infiltrating the ischemic myocardium, and reaches high levels in the circulation post-MI. A potent S100A8/A9 response seems to be deleterious in this context, as elevated systemic S100A8/A9 has been associated with a negative long-term prognosis in MI patients.

It has earlier been recognized that an excessive inflammatory response to MI amplifies myocardial injury, leading to larger infarctions and loss of function. However, clinical trials testing anti-inflammatory strategies in MI patients have so far led to non-significant or deleterious effects. Ideally, an efficient therapy in MI should inhibit the damaging effects of excessive inflammation, while leaving the repair mechanisms intact. Currently, coronary revascularization, i.e. opening of occluded or significantly stenosed coronary vessels by balloon angioplasty (with or without stenting) or by coronary-artery by-pass grafting, is the cornerstone of patient management in MI according to guidelines. This procedure is performed acutely in STEMI patients, where the coronary artery is still occluded upon hospital presentation in the majority of cases. As spontaneous coronary reperfusion occurs in the majority of NSTEMI patients, coronary angiography and stenting is usually performed within 24-48 hours. Other guidelines-recommended treatments are dual anti-platelet therapy with acetylsalicylic acid and P2Y12 inhibitors to prevent further thrombotic events, lipid-lowering therapy with statins, angiotensin-converting enzyme inhibitors and beta blockers to reduce left ventricular pressure and prevent excessive fibrosis. However, none of these treatments directly address the pathological inflammation that occurs in the myocardium. The myocardial infarction caused by myocardial ischemia and ischemia/reperfusion injury is associated with myocardial inflammation, which further contributes to myocardial damage and to complications such as heart failure, arrhythmias and myocardial rupture. A treatment able to inhibit the excessive inflammation that occurs in the myocardium could potentially reduce the myocardial damage, improve cardiac function, lower the rate of post-MI complications and improve both short-term and long-term prognosis in MI patients.

Sepsis is a severe, life-threatening disease characterized by a potent immune and inflammatory response to an infection, leading to organ dysfunction. Cardiovascular depression, dysfunction of the cardiovascular system, is the leading cause of death in sepsis. The hallmarks of cardiovascular depression in sepsis include peripheral vascular dysfunction as well as cardiac dysfunction, termed septic cardiomyopathy (i.e. sepsis-induced cardiomyopathy). The innate immune system plays a central role in the pathogenesis of sepsis. Bacterial products such as lipopolysaccharide (LPS) act as DAMPs, triggering potent local and systemic immune responses. Bacterial LPS induces inflammation in the myocardium, characterized, among others, by upregulation of the classic pro-inflammatory cytokines TNF-a, IL-1b and IL-6. Markedly increased levels of S100A8 and S100A9 in the heart and blood of animals and humans challenged with bacteria and bacterial LPS have also been observed. Treatment with antibiotics, fluid and vasopressor therapy are the cornerstones of the current treatment of sepsis patients. However, mortality rates in sepsis have not improved during the past years, calling for novel therapies to be developed that are able to inhibit the excessive immune and inflammatory reaction in sepsis.

In myocarditis, the acute myocardial damage is triggered, among others, by viruses, bacteria, autoantibodies or activated leukocytes. Myocardial injury due to myocarditis might occur in systemic autoinflammatory diseases such as systemic lupus erythematosus, rheumatoid arthritis or sarcoidosis. Although unspecific anti-inflammatory regimes using combinations of steroids, intravenous immunoglobulins, azathioprine, and cyclosporine A have been tried in certain forms of myocarditis, currently there are no established guidelines-recommended anti-inflammatory treatments in myocarditis.

As discussed above, currently there are no available treatments able to specifically inhibit the inflammatory damage to the heart that occurs in myocardial infarction secondary to myocardial ischemia or ischemia/reperfusion, myocarditis, sepsis, sepsis-induced myocardial inflammation and septic cardiomyopathy. This is an important clinical problem, as myocardial infarction is the most frequent cause of death among non-communicable diseases, and acute myocarditis leads in 25% of cases to persistent cardiac dysfunction, death or progression to end-stage dilated cardiomyopathy in need of heart transplantation. In addition, septic cardiomyopathy is a major contributor to mortality in sepsis patients. Mortality can occur in up to 30% of patients, making sepsis the second most common cause of death in noncoronary intensive care units.

### SUMMARY

As described above, a treatment capable of modulating myocardial inflammation is highly desirable as it could potentially reduce the myocardial damage and improve cardiac function and thereby prognosis in patients having a disease or disorder involving myocardial inflammation, such as MI patients or patients with myocarditis or sepsis. Furthermore, an adequate balance between the two phases of the immune response to myocardial infarction (MI), the acute or inflammatory phase and the reparatory phase, is crucial for efficient myocardial repair, recovery of cardiac function and a favorable patient prognosis.

The present disclosure relates to a compound according to formula (I) for use in the treatment of a disease or disorder involving myocardial inflammation. The inventors of the present disclosure have surprisingly found that treatment of a disease or disorder involving myocardial inflammation, such as myocardial infarction due to myocardial ischemia or ischemia/reperfusion, myocarditis and sepsis, using said compound results in reduced cardiomyocyte damage, improved myocardial repair and recovery of cardiac function.

More specifically, the inventors have found that a beneficial balance between the two phases of the immune response to MI is obtained by treatment with said compound during the acute phase of the immune response to MI, by reducing the deleterious effects of the inflammatory response induced by the S100A8/A9 complex and/or its components. In addition, the inventors have found that continued treatment with said compound in the reparatory phase reduces the presence of reparatory cells in the myocardium, which negatively interferes with the proper recovery of the heart. Thus, the inventors have found a surprisingly advantageous effect in only administering said compound during the acute phase following an MI and discontinuing the treatment in the reparatory phase, which leads to improved heart function. These findings enable a completely new way to address the treatment of acute inflammation in the myocardium after MI, which could potentially lead to improved patient recovery and reduced risk of heart failure. Additionally, the inventors have found that treatment with the compound of the disclosure in the acute phase of sepsis leads to lower levels of systemic inflammation, significantly improved cardiac function, reduced weight loss and improved survival. Also, treatment with said compound in the acute phase of myocarditis leads to reduced inflammatory cell infiltration into the heart and heart-draining lymph nodes, and improved cardiac function.

The present disclosure thus provides a compound according to formula (I) for use in treatment of acute inflammation in the myocardium. Particularly, the present disclosure relates to a compound according to formula (I) for use in the treatment of a disease or disorder involving myocardial inflammation.

Thus, in one aspect, a compound of formula (I) is provided:

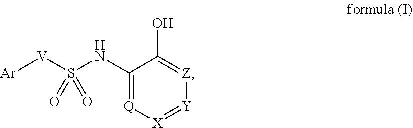

wherein
W is CH or N; X is N or CR¹; Y is CR² or N; Z is CR³ or N; at least one and at most two of W, X, Y, and Z are N;
R¹ is H, halogen, S(O)₂C1-C3 alkyl, cyano, or C1-C3 alkyl optionally substituted with one or more halogen(s);
R² is H, halogen, cyano, C(O)OH, C(O)OC1-C3 alkyl, C1-C3 alkyl optionally substituted with one or more F; hydroxy-C1-C3 alkyl, S(O)₂C1-C3 alkyl, S(O)₂C3-C6 cycloalkyl or S(O)₂C1-C3 hydroxyalkyl;
R³ is H, halogen or cyano;
V is (CHR⁴)ₘ;
m is 0 or 1;
R⁴ is H or C1-C3 alkyl optionally substituted with one or more halogen(s);

### Ar is

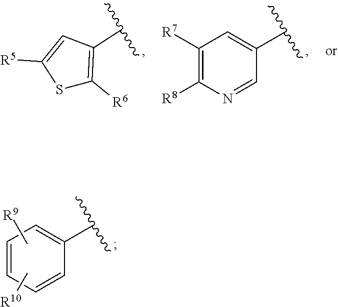

R⁵ is halogen,
H, or cyano;
R⁶ is halogen, or H;
R⁷ is halogen, H, C1-C3 alkyl, cyano, S(O)₂C1-C3 alkyl, or phenyl;
R⁸ is C1-C3 alkoxy optionally substituted with one or more F, C1-C3 alkyl optionally substituted with one or more F, H, halogen, phenoxy, NHR¹¹, or NR¹¹R¹²;
R⁹ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹⁰ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹¹ is C1-C3 alkyl;
R¹² is C1-C3 alkyl; or
R¹¹ and R¹², together with the nitrogen atom to which they are both attached, form a ring of formula

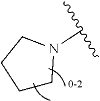

R¹³ is H or C1-C3 alkyl; and
R¹⁴ is H or C1-C3 alkyl;
or a pharmaceutically acceptable salt thereof, for use in the treatment of a disease or disorder involving myocardial inflammation.

More particularly, a compound for formula (Ia), formula (Ib), formula (Ic), formula (Id), formula (Ie), or formula (If) is provided:

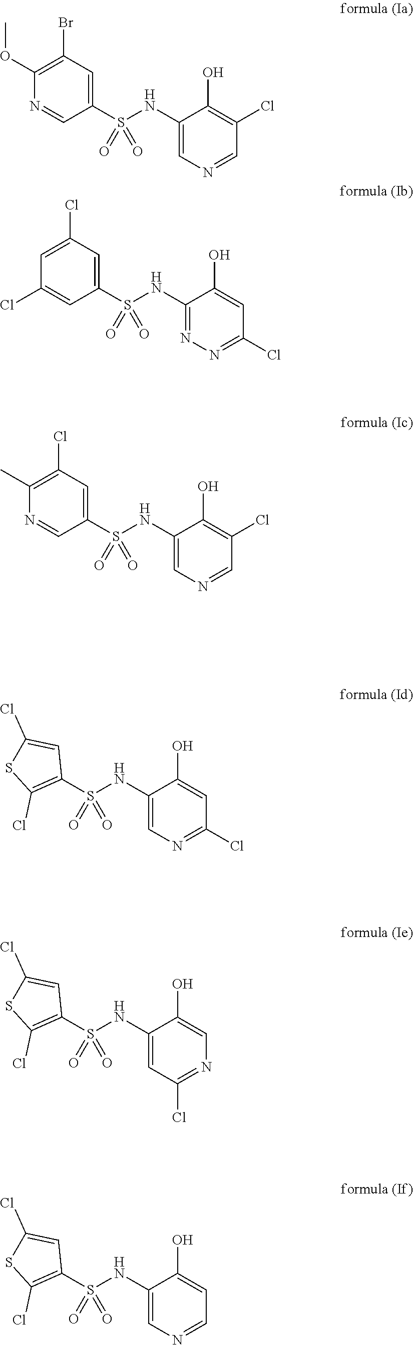 [SMILES: COc1ncc(S(=O)(=O)Nc2cncc(Cl)c2O)cc1Br ; O=S(=O)(Nc1nnc(Cl)cc1O)c1cc(Cl)cc(Cl)c1 ; Cc1ncc(S(=O)(=O)Nc2cncc(Cl)c2O)cc1Cl ; O=S(=O)(Nc1cnc(Cl)cc1O)c1cc(Cl)sc1Cl ; O=S(=O)(Nc1cc(Cl)ncc1O)c1cc(Cl)sc1Cl ; O=S(=O)(Nc1cnccc1O)c1cc(Cl)sc1Cl]

or a pharmaceutically acceptable salt thereof, for use in the treatment of a disease or disorder involving myocardial inflammation.

In one embodiment, the present invention relates to treatment of a disease or disorder involving myocardial inflammation by administration of said compound during the acute phase of the disease or disorder.

### DESCRIPTION OF DRAWINGS

FIG. 1: Short-term S100A9 blockade during the inflammatory phase improves cardiac function post-MI.

(A) Experimental layout: C57BL/6 mice with MI induced by permanent coronary artery ligation received three daily i.p. injections of the S100A9 blocker ABR-238901 (ABR, 5-bromo-N-(5-chloro-4-hydroxypyridin-3-yl)-6-methoxypyridine-3-sulfonamide, formula (Ia)) or PBS, starting immediately after the MI and administered on days 0, 1 and 2 after the coronary artery ligation. Cardiac function was assessed by echocardiography at baseline and on days 3, 7 and 21 in the Sham (n=5), MI (n=9) and MI+3d ABR (n=12) groups. (B) Left ventricular ejection fraction (EF) was significantly improved by the ABR treatment at all considered time points, in the MI+ABR 3d group compared to the MI group. Cardiac function was not affected in the Sham group. (C) Micrographs showing lower scar size in the ABR-treated group compared to untreated mice, determined by Van Gieson collagen staining of heart slices collected at day 21 post-MI. Scar size was quantified as percentage of left ventricle volume. (D) Representative magnetic resonance (MR) short-axis and long-axis still images of the left ventricle in mice from the MI and MI+3d ABR groups, recorded at 3, 7 and 21 days post-MI. The experiment included three groups: sham (n=9), MI (n=8) and MI+3d ABR (n=8). (E) Left ventricular ejection fraction and (F) Cardiac output were significantly improved in mice receiving ABR treatment for 3 days post-MI. (G) End-systolic volume, and (H) End-diastolic volume were lower in ABR-treated mice, suggesting lower cardiac remodeling. (1) Infarction size was lower at 3 and 7 days post-MI in the MI+3d ABR group compared to the MI group, measured by the TTC method and expressed as percentage of the area-at-risk (AAR). (J) TUNEL staining showing decreased myocardial presence of apoptotic cells in the ABR-treated mice on day 3 post-MI. The P-values for the repeated measurement experiments refer to the difference between the MI and the MI+3d ABR groups, analysed by 2-way ANOVA with Fisher LSD post-hoc evaluation. Data is presented as mean±SD. The differences in scar and infarction size between the groups were assessed with the Student's T-test. Only the significant P-values are shown.

FIG. 2: Short-term S100A9 blockade blunts the inflammatory response to MI.

(A-C) Neutrophil counts in blood, spleen and bone marrow in unoperated and untreated mice (Steady state) and in mice with induced MI treated for 2 days i.p. with PBS (MI) or ABR (MI+ABR). (D-F) Total monocyte counts in blood, spleen and bone marrow in the Steady state, MI and MI+ABR groups. The ABR treatment led to lower numbers of neutrophils in blood, and increased presence of neutrophils and monocytes in the bone marrow, compared with mice with MI treated with PBS. The Student's T-test was used to analyse statistical differences between the groups. (G) Gating strategy and numbers of proliferating BrdU⁺ Lin⁻Sca⁺Kit⁺CD150⁺CD48⁻ hematopoietic stem cell (HSC) in the bone marrow. BrdU was injected i.v. 24 h prior to the analysis. The ABR treatment inhibited HSC proliferation in the bone marrow post-MI. The Student's T-test was used to analyse statistical differences between the groups. Total numbers of neutrophils (H), monocytes (I), and CD11b⁺F4/80⁺ macrophages (J) in the heart in steady-state, MI and MI+3d ABR groups. The ABR treatment reduced the infiltration of neutrophils and monocyte/macrophages in the heart on day 2 post-MI. N=5 in all groups. Data is presented as mean±SD. The Student's T-test was used to analyse statistical differences between the groups.

FIG. 3: Short-term S100A9 blockade tips the inflammation/repair balance towards a reparatory environment in the ischemic myocardium.

Myocardial mRNA expression of pro-inflammatory (A-D) and reparatory (E-I) genes measured at 3 days after MI in the sham, MI, and MI+3d ABR groups. (j) Cardiac mRNA expression of the chemotactic factor CCL5. mRNA values for the respective genes have been normalized to the expression of hypoxanthine-guanine phosphoribosyltransferase (HPRT) in the tissue. (k) Fold change of gene expression in the myocardium of ABR-treated mice compared to MI controls at 3 days after coronary artery ligation. ABR treatment induced lower transcription of inflammatory genes and higher transcription of reparatory genes in the myocardium compared to PBS-treated mice with MI. (I) S100A8/A9 levels in plasma at 3 days post-MI in the sham (n=3), MI (n=11) and MI+3d ABR (n=12) groups. (m) S100A8/A9 presence in the myocardium 3 days after MI, determined by immunohistochemical staining. S100A9 blockade with ABR reduced the levels of S100A8/A9 both in plasma and in the myocardium. N=5 mice per group, except for (I). Data is presented as mean±SD. The Student's T-test was used to analyse statistical differences between the groups.

FIG. 4: Long-term S100A9 blockade impairs myocardial repair, leading to increased cardiac remodelling and loss of function.

(A) C57BL/6 mice with permanent coronary artery ligation were treated with ABR or buffer for 21 days. (B-D) Cardiac function and volumes determined by echocardiography (n=3 in the sham groups and n=14-18 in the MI groups) demonstrated decreased left ventricular ejection fraction and increased end-systolic and end-diastolic volumes in mice with MI receiving long-term ABR treatment beyond the acute phase of the disease. (E) Mason trichrome collagen staining of heart slices at day 21 post-MI, showing increased scar size in ABR-treated mice with MI. The 2-tailed Student's T-test was used for statistical calculations. Data are presented as mean±SEM, *P<0.05, **P<0.01, ***P<0.001. (F-H) Neutrophil counts in blood, spleen and bone marrow and (I) monocyte counts in blood in mice with induced MI treated with ABR or PBS i.p. for 7 days. The 2-tailed Student's T-test was used for statistical calculations. Data are presented as mean±SEM, *P<0.05, **P<0.01, ***P<0.001. (J, K) Monocyte counts in spleen and bone marrow in mice with induced MI treated with ABR or PBS i.p. for 7 days. Numbers of neutrophils (I), monocytes in the heart after 7 days of ABR or PBS treatment post-MI. ABR treatments led to lower neutrophil numbers in blood. Monocyte numbers were lower in blood and heart, but higher in the spleen of mice receiving ABR. The 2-tailed Student's T-test was used for statistical calculations. Data are presented as mean±SEM, *P<0.05, **P<0.01, ***P<0.001. Total CD11b⁺F4/80⁺ macrophages (N), and reparatory MerTK^{hi}Ly6C^{lo} macrophages (O) in the heart after 7 days of ABR or PBS treatment post-MI. (P) Percentage of reparatory macrophages out of total macrophages in the heart. (Q-R) Numbers of mCherry+ macrophages and mCherry mean-fluorescence intensity (MFI) in macrophages collected from the hearts of transgenic mice expressing the fluorescent protein mCherry in cardiomyocytes, treated with ABR or PBS for 7 days post-MI. The ABR treatment reduced the presence of total macrophages in the heart, as well as the numbers and percentages of reparatory macrophages and of reparatory macrophages having phagocytosed dead cardiomyocytes. (S-T) The levels of the pro-reparatory transcription factor Nur77, measured by flow cytometry MFI, was lower in blood Ly6C^{hi/int} monocytes collected from C57BL/6 mice reconstituted with C57BL/6 Nur77-EGFP bone marrow and treated with ABR (n=6) for 7 days post-MI, as compared to PBS-treated mice (n=6). The 2-tailed Student's T-test was used for statistical calculations. Data are presented as mean±SEM, *P<0.05, **P<0.01, ***P<0.001. (U) Nur77 MFI in cultured bone marrow-derived monocytes 24h after stimulation with S100A9 in the presence or absence of ABR (n=5). (V) Renilla luciferase reporter activity in mouse RAW264.7 macrophages transfected with either of the two main Nur77 response elements, NBRE or NurRE, and treated with S100A9 and/or ABR (n=6). Stimulation with S100A9 increased the levels and activity of Nur77 in monocytes and macrophages, and ABR reversed these effects. (W) The numbers of reparatory MerTK^{hi}Ly6C^{lo} macrophages in the heart 7 days post-MI were significantly reduced in mice treated with ABR for 7 days, but were unaffected in MI mice receiving short-term S100A9 blockade for only 3 days. The 2-tailed Student's T-test was used for statistical calculations. Data are presented as mean±SEM, *P<0.05, **P<0.01, ***P<0.001.

FIG. 5: Short-term S100A9 blockade improves cardiac function after myocardial ischemia/reperfusion.

(a) C57BL/6 mice with MI induced by coronary artery ligation for 60 minutes followed by reperfusion received three daily i.p. injections of ABR or PBS, starting at the time of reperfusion and administered on days 0, 1 and 2 post-MI. Cardiac function was assessed by echocardiography the day before surgery and on days 3, 7, 14 and 21 in the Sham (n=10), Sham+ABR (n=10), MI (n=22) and MI+3d ABR (n=13) groups. (B) Left ventricular ejection fraction (EF) and (C) Cardiac output were significantly improved by the short-term ABR treatment. (D) End-systolic volume and (E) End-diastolic volume were not significantly affected by the treatment. (F) Neutrophil and (G) monocyte counts in the bone marrow were increased 2 days after myocardial ischaemia/reperfusion in the MI+3d ABR group (n=6) compared to the MI group treated with PBS (n=6). (H) Neutrophil, (I) monocyte, and (J) macrophage numbers in the myocardium in the MI and MI+3d ABR groups on Day 2 post-MI. Macrophage presence in the heart was significantly reduced by the S100A9 blockade with ABR. The P-values refer to the difference between the MI and the MI+3d ABR groups. The P-values for the repeated measurement experiments refer to the difference between the MI and the MI+3d ABR groups, analysed by 2-way ANOVA with Fisher LSD post-hoc evaluation. Only the significant P-values are shown. Data is presented as mean±SD.

FIG. 6: Incident heart failure post-acute coronary syndrome, by baseline S100A8/A9 tertiles.

Kaplan-Meier diagram of incident heart failure during follow-up in a population of 524 acute coronary syndrome patients divided by baseline S100A8/A9 tertiles measured within 24 hours after hospital admission. The patient cohort included 474 patients (90.5%) with myocardial infarction, and 50 patients (9.5%) with unstable angina. The data demonstrate highly increased risk to develop heart failure in patients with S100A8/A9 levels in the highest tertile. The P value for the trend was calculated with an unadjusted log-rank test.

FIG. 7: ABR-238901 inhibits binding of S100A9 to RAGE and TLR4.

(a) The molecular structure of ABR-238901. (b-c) Surface plasmon resonance measurement of S100A9 binding to immobilized RAGE (b) and TLR4 (c) in the presence of increasing ABR-238901 concentrations. The magnitude of the response for each ABR-238901 concentration (BABR-238901), measured at the late dissociation phase (t 350s), was expressed as percentage of the response in the absence of competitor (BO), and plotted against the concentration of ABR-238901.

FIG. 8: Gating strategy for flow cytometry analyses of cellular populations in heart (A), blood (B), spleen (C), and bone marrow (D).

LSKs (Lin⁻Sca-1⁺c-kit⁺) hematopoietic stem and progenitor cells; HSCs, hematopoietic stem cells; MPPs, multipotent progenitor cells; LRPs, lineage-restricted progenitors.

FIG. 9: Effects of S100A9 blockade on monocyte subpopulations 2 days post-MI.

Number of Ly6C^{hi/int} and Ly6C^{lo} monocyte subpopulations in the blood (A), heart (B) and spleen (C) of C57BL/6 mice with induced MI after 2 days treatment with ABR-238901 (MI+ABR) or PBS (MI). The number of Ly6C^{hi} monocytes in the bone marrow were increased by the ABR treatment (D). The treatment was started at the time of the MI (n=5 per group). Data are represented as mean±SEM. * P<0.05.

FIG. 10: Effects of S100A9 blockade on monocytes subpopulations in different tissues 7 days following myocardial infarction.

Number of Ly6C^{hi/int} and Ly6C^{lo} monocyte subpopulations in the blood (A), heart (B) and spleen (C), and number of Ly6Chi monocytes in the bone marrow (D) of C57BL/6 mice with induced MI after 7 days treatment with ABR-238901 (MI+ABR) or PBS (MI). Both monocyte subpopulations were reduced in blood and heart, and were increased in the spleen of mice receiving S100A9 blockade with ABR. The treatment was started at the time of the MI (n=5 per group). Data are represented as mean±SEM. * P<0.05.

FIG. 11: Effects of S100A9 blockade on heart macrophages at 7 days post-MI in C57BL/6 mice reconstituted with Nur77-EGFP bone marrow.

(a) The numbers of F4/80⁺Ly6C^{lo}MerTK^{hi} reparatory macrophages in the heart were reduced by the ABR treatment. (b) Nur77 MFI in heart F4/80⁺Ly6C^{lo}MerTK^{hi} reparatory macrophages was unchanged.

FIG. 12: The role of S100A9 in the innate immune response involved in post-MI inflammation and repair.

(a) In the inflammatory phase, S100A9 stimulates HSC proliferation in the bone marrow, as well as neutrophil and monocyte egression into the blood stream and recruitment into the ischemic myocardium. Short-term blockade of S100A9 dampens the inflammatory response to MI, leading to smaller infarctions and improved cardiac function. (b) In the reparatory phase, S100A9 promotes monocyte egression from the spleen and their recruitment into the myocardium. S100A9 also stimulates the transition of Ly6C^{hi} monocytes into reparatory Ly6C^{lo} macrophages by activating the transcription factor Nur77. Long-term S100A9 blockade reduces the numbers of reparatory MerTK^{hi}Ly6C^{lo} macrophages in the myocardium, finally leading to impaired cardiac repair and function.

FIG. 13: S100A9 blockade with ABR-238901 improves cardiac function in sepsis.

(A) Experimental layout: Sepsis was induced in female C57BL/6 mice (10 weeks of age) by intraperitoneal (i.p.) injection of bacterial lipopolysaccharide (LPS), 5 mg/kg. The mice were subsequently treated with two doses of 30 mg/kg ABR-238901 or with an equivalent volume of PBS, administered i.p. immediately after the LPS injection and 6 h later (n=5 mice per group). Cardiac function was evaluated by echocardiography at baseline and at 6, 12 and 24 hours after the induction of sepsis. (B) Left ventricular ejection fraction was significantly improved by S100A9 blockade (12 h—P=0.0157; 24 h—P=0.0008). (C) Cardiac output was also improved in mice receiving ABR-238901 treatment (24 h—P=0.064). Abbreviations: Echo, Echocardiography; LV, Left Ventricle. * P<0.05, *** P<0.001. A two-tailed unpaired Student's T-test was used to compare left ventricular ejection fraction and cardiac output between the groups at a given timepoint. Data is presented as mean±SEM.

FIG. 14: S100A9 blockade with ABR-238901 reduces mortality in sepsis.

(A) Experimental layout: Sepsis was induced in female C57BL/6 mice (10 weeks of age) by intraperitoneal (i.p.) injection of bacterial lipopolysaccharide (LPS), 5 mg/kg. The mice were subsequently treated with two doses of 30 mg/kg ABR-238901 or with an equivalent volume of PBS, administered i.p. immediately after the LPS injection and 6 h later (n=9 mice in the “Sepsis+ABR-238901” group; n=10 mice in the “Sepsis” group treated with PBS). The mice were followed for 7 days to evaluate mortality. (B) Kaplan-Meier curves demonstrating reduced animal mortality in the sepsis group receiving ABR-238901 (P=0.038). A log-rank (Mantel Cox) test was performed to compare survival in the 2 groups during the 7 days follow-up period.

FIG. 15: S100A9 blockade with ABR-238901 ameliorates weight loss during sepsis.

(A) Experimental layout: Sepsis was induced in female C57BL/6 mice (10 weeks of age) by intraperitoneal (i.p.) injection of bacterial lipopolysaccharide (LPS), 5 mg/kg. The mice were subsequently treated with two doses of 30 mg/kg ABR-238901 or with an equivalent volume of PBS, administered i.p. immediately after the LPS injection and 6 h later (n=5 mice per group). The weight of the mice was registered daily for 7 days. (B) Graph illustrating weight loss expressed as percent of baseline weight during the experiment. The mice treated with ABR-238901 had a significantly lower decline in bodyweight following induction of sepsis (Day 1, P=0.0007; Day 2, P=0.0145; Day 3, P=0.00002; Day 4, P=0.034). * P<0.05; *** P<0.001; **** P<0.0001. A two-tailed unpaired Student's T-test was used to compare weight loss between the groups at a given time point. Data are presented as mean±standard error of the mean (SEM).

FIG. 16: S100A9 blockade with ABR-238901 lowers plasma concentration of pro-inflammatory cytokines in sepsis.

(A) Experimental layout: Sepsis was induced in female C57BL/6 mice (10 weeks of age) by intraperitoneal (i.p.) injection of bacterial lipopolysaccharide (LPS), 5 mg/kg. The mice were subsequently treated with two doses of 30 mg/kg ABR-238901 or with an equivalent volume of PBS, administered i.p. immediately after the LPS injection and 6 h later (n=5 mice per group). The mice were sacrificed at 24 h after sepsis induction and blood was collected through cardiac puncture. Plasma was isolated for measurement of soluble factors. (B) Graphs showing the levels of pro-inflammatory cytokines in plasma of mice with sepsis treated with ABR-238901 or PBS. The soluble mediators have been measured by a multiplex cytokine assay and expressed in pg/mL in all graphs. Compared with the control PBS group, treatment with ABR-238901 lowered a wide range of pro-inflammatory cytokines and chemokines in septic mice (IL-1b, P=0.031; IL-6, P=0.037; IL17-A, P=0.02; IL-22, P=0.0092; IFN-γ, P=0.03; MCP-1, P=0.04; LIX, P=0.01; LIF, P=0.03; GM-CSF, P=0.04). Dark grey bars represent mice in the sepsis group treated with PBS, light grey bars represent mice in the sepsis group treated with ABR-238901. * P<0.05: ** P<0.01. A two-tailed unpaired Student's T-test was used to compare mediator levels between groups. Data are presented as mean±standard deviation (SD).

FIG. 17: S100A9 blockade with ABR-238901 improves cardiac function in myocarditis.

(A) Experimental layout: myocarditis was induced in female BALB/c mice (8 weeks of age) by subcutaneous administration of 100 μg cardiac α-myosin peptide emulsified in complete Freund's adjuvant at day 0 and day 7. ABR-238901 was administered orally ad-libitum diluted in meglumine buffer at a dose of 30 mg/kg/day, starting on day 7 and throughout the course of the experiment (n=9). Fluid intake was closely monitored during the experiment to ensure an appropriate drug dosage. Mice with induced myocarditis drinking meglumine buffer alone were used as controls (n=12). Cardiac function was evaluated by echocardiography at baseline, 21, 28 and 35 days. (B) ABR-238901 treatment led to improved left ventricular ejection fraction in mice with myocarditis as compared to controls receiving meglumine buffer alone (Day 21, P=0.056; Day 28, P=0.013; Day 35, P=0.035). (C) Cardiac output was also significantly improved in mice receiving ABR-238901 (Day 21 P=0.077; Day 28, P=0.027; Day 35, P=0.034). Abbreviations: Echo, Echocardiography; LV, Left Ventricle. * P<0.05. A two-tailed unpaired Student's t-test was used to compare parameters of cardiac function in the two groups at a given timepoint. Data is presented as mean±SEM.

FIG. 18: S100A9 blockade with ABR-238901 inhibits the infiltration of inflammatory cells in the heart and heart-draining lymph nodes during myocarditis.

(A) Experimental layout: myocarditis was induced in female BALB/c mice (8 weeks of age) by subcutaneous administration of 100 μg cardiac α-myosin peptide emulsified in complete Freund's adjuvant at day 0 and day 7. ABR-238901 was administered orally ad-libitum diluted in meglumine buffer at a dose of 30 mg/kg/day, starting on day 7 and throughout the course of the experiment (n=10 mice per group). The mice were sacrificed on day 21, and hearts and mediastinal heart-draining lymph nodes were isolated. Single cell suspensions were obtained from the organs and stained with fluorescently-conjugated antibodies for analysis by flow cytometry. (B) We found reduced myocardial infiltration of conventional dendritic cells (cDCs; P=0.008) and of eosinophils (P=0.03, FIG. 18C), expressed as percent of live CD45⁺ leukocytes, in mice receiving ABR-238901 treatment. (D-F) The presence of pro-inflammatory pathogenic T cells in the heart-draining lymph nodes of mice with myocarditis was markedly reduced by the ABR-238901 treatment. (D) Total CD3⁺ T cells, expressed as percentage of live CD45⁺ leukocytes (P=0.0518). (E) Effector memory CD4⁺ T cells (defined as CD4⁺CD44⁺CD62L⁻ cells) expressed as percent of live CD45⁺CD3⁺ T cells (P<0.0001). (F) Effector memory CD8⁺ T cells (defined as CD8⁺CD44⁺CD62L⁻ cells) expressed as percent of live CD45⁺CD3⁺ T cells (P=0.0001). Abbreviations: cDCs, conventional dendritic cells; Myo, Myocarditis; ABR, ABR-238901; EM, Effector Memory. * P<0.05; ** P<0.01; *** P<0.001. A two-tailed unpaired Student's T-test was used for the statistical analyses. Data are presented as mean±SD.

### DETAILED DESCRIPTION

The present disclosure relates to a compound according to formula (I) for use in the treatment of a disease or disorder involving myocardial inflammation, such as for example myocardial infarction due to myocardial ischemia or myocardial ischemia/reperfusion, myocarditis, sepsis, sepsis-induced myocardial inflammation and/or septic cardiomyopathy. Treatment of such diseases by administration of said compound is herein demonstrated to provide reduced systemic and cardiac inflammation, improved cardiac function, reduced myocardial damage and/or improved myocardial repair. More specifically, treatment of myocardial infarction by administration of said compound during the acute phase of the disease, while discontinuing the treatment during the reparatory phase, is demonstrated to provide reduced systemic and cardiac inflammation, improved myocardial repair and recovery of cardiac function. Furthermore, administration of said compound during the acute phase of sepsis improves cardiac function, inhibits inflammation, and improves mortality. In myocarditis, administration of said compound during the acute phase of the disease reduces inflammation in the myocardium and in the draining lymph nodes, and improves cardiac function.

### Definitions

The term “alkyl” as used herein, refers to straight or branched chain alkyl of the general formula CₙH₂ₙ₊₁

The term “C1-C3 alkyl” as used herein, refers to a straight or branched alkyl, such as for example methyl, ethyl, n-propyl and isopropyl.

The term “halogen” as used herein, refers to F, Cl, Br or I.

The term “hydroxyl” as used herein, refers to a radical of the formula —OH.

The term “hydroxy-C1-C3 alkyl” as used herein refers to an alkyl radical substituted with a hydroxy, e.g. 1-hydroxypropan-2-yl.

The term “C1-C3 alkylthio” as used herein refers to a radical of the formula —SR, wherein R is C1-C3 alkyl.

The term “C1-C3 alkoxy” as used herein, refers to a radical of the formula —OR, wherein R is C1-C3 alkyl.

The term “phenoxy” as used herein, refers to a radical of the formula —OR wherein R is phenyl.

The term “cyano” as used herein, refers to a radical of formula —C≡N (i.e. —CN).

The term S(O)₂C1-C3 alkyl refers to a radical of formula

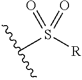

wherein R is C1-C3 alkyl.

The term S(O)₂C3-C6 cycloalkyl refers to a radical of formula

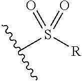

wherein R is C3-C6 cycloalkyl.

The term S(O)₂C1-C3 hydroxyalkyl refers to a radical of formula

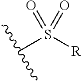

wherein R is C1-C3 hydroxyalkyl.

The term C(O)OC1-C3 alkyl refers to a radical of formula

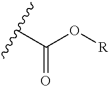

wherein R is C1-C3 alkyl.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

The term “pharmaceutically acceptable salt” of a compound refers to a salt that is pharmaceutically acceptable, as defined herein, and that possesses the desired pharmacological activity of the parent compound. Pharmaceutically acceptable salts include acid addition salts formed with inorganic acids, e.g. hydrochloric acid, hydrobromic acid, sulfuric acid, nitric acid, phosphoric acid; or formed with organic acids, e.g. acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, ptoluenesulfonic acid, trimethylacetic acid; or salts formed when an acidic proton present in the parent compound either is replaced by a metal ion, e.g., an alkali metal ion, an alkaline earth ion, or an aluminum ion; or coordinates with an organic or inorganic base. Acceptable organic bases include e.g. diethanolamine, ethanolamine, N-methylglucamine, triethanolamine, morpholine, and tromethamine. Acceptable inorganic bases include e.g. ammonia, aluminum hydroxide, calcium hydroxide, potassium hydroxide, sodium carbonate and sodium hydroxide.

Whenever a chiral carbon is present in a chemical structure, it is intended that all stereoisomers associated with that chiral carbon are encompassed by the structure, unless otherwise specified. Using the Cahn-Ingold-Prelog RS notational system, any asymmetric carbon atom may be present in the (R)- or (S)-configuration, and the compound may be present as a mixture of its stereoisomers, e.g. a racemic mixture, or one stereoisomer only.

Some of the compounds of the invention may exist in tautomeric forms, e.g. 2-hydroxypyridine and its tautomer 2-pyridone. Any such tautomer is contemplated to be within the scope of the invention.

Also, in a compound of formula (I) as defined herein, any hydrogen atom may be replaced by a deuterium (²H), and any such deuterated compound of formula (I), comprising one or more deuteriums in place of the corresponding number of hydrogen atoms, is considered to be within the scope of the invention.

The compounds of formula (I) carry a hydroxy group on the ring containing W, X, Y and Z. It has been found that the corresponding alkoxy compounds, i.e. where said hydroxyl group is alkylated, are prodrugs of the compounds of formula (I). “Therapeutically effective amount” means an amount of a compound that, when administered to a subject for treating a disease state, is sufficient to effect such treatment for the disease state. The “therapeutically effective amount” will vary depending on the compound, the disease state being treated, the severity of the disease treated, the age and relative health of the subject, the route and form of administration, the judgment of the attending medical or veterinary practitioner, etc. As used herein the terms “treatment” or“treating” is an approach for obtaining beneficial or desired results including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total) whether detectable or undetectable. The term can also mean prolonging survival as compared to expected survival without the treatment.

By “Type 1 myocardial infarction” is meant a myocardial infarction characterized by detection of a rise and/or fall of cardiac troponin values with at least one value above the 99^{th} percentile upper reference limit, and with at least one of the following: symptoms of acute myocardial ischemia; new ischemic ECG changes; development of pathological Q waves; imaging evidence of new loss of viable myocardium or new regional wall motion abnormality in a pattern consistent with an ischemic aetiology; or identification of a coronary thrombus by angiography including intracoronary imaging or by autopsy.

By “Type 2 myocardial infarction” is meant a myocardial infarction characterized by detection of a rise and/or fall of cardiac troponin values with at least one value above the 99^{th} percentile upper reference limit, and evidence of an imbalance between myocardial oxygen supply and demand unrelated to acute coronary atherothrombosis, requiring at least one of the following: symptoms of acute myocardial ischemia; new ischemic ECG changes; development of pathological Q waves; or imaging evidence of new loss of viable myocardium or new regional wall motion abnormality in a pattern consistent with ischemic aetiology.

By “Type 4 myocardial infarction” is meant a coronary intervention-related myocardial infarction characterized by an elevation of cardiac troponin values more than five times the 99^{th} percentile upper reference limit in patients with normal baseline values. In patients with elevated pre-procedure cardiac troponin in whom the cardiac troponin levels are stable (s 20% variation) or falling, the post-procedure cardiac troponin must rise by >20%. The absolute post-procedural value must still be at least five times the 99^{th} percentile upper reference limit. In addition, one of the following elements is required: new ischemic ECG changes; development of new loss of viable myocardium or new regional wall motion abnormality in a pattern consistent with an ischemic aetiology; angiographic findings consistent with a procedural flow-limiting complication such as coronary dissection, occlusion of a major epicardial artery or a side branch occlusion/thrombus, disruption of collateral flow, or distal embolization.

By “Type 5 myocardial infarction” is meant a CABG-related myocardial infarction characterized as an elevation of cardiac troponin values >10 times the 99^{th} percentile upper reference limit in patients with normal baseline cardiac troponin values. In patients with elevated pre-procedure cardiac troponin in whom cardiac troponin levels are stable (s 20% variation) or falling, the post-procedure cardiac troponin must rise by >20%. However, the absolute post-procedural value must still be >10 times the 99^{th} percentile upper reference limit. In addition, one of the following elements is required: development of new pathological Q waves; angiographic documented new graft occlusion or new native coronary artery occlusion; imaging evidence of new loss of viable myocardium or new regional wall motion abnormality in a pattern consistent with an ischemic aetiology.

By “ST-elevation myocardial infarction” or “STEMI” is meant a myocardial infarction characterized by an abnormally high ST-segment on the ECG. A STEMI may be linked to the myocardium having suffered a transmural infarction. In some cases, a STEMI may be linked to the myocardium having suffered a subendocardial infarction.

By “non-ST-elevation myocardial infarction” or “NSTEMI” is meant a myocardial infarction which is in part characterized by for example the absence of an ST-segment elevation in the ECG. NSTEMI may be linked to the myocardium having suffered a subendocardial infarction.

By “revascularization” is meant the surgical or medical restoration of blood perfusion to a body part or organ that has suffered ischemia. Vascular bypass surgery and vascular angioplasty are the two primary means of surgical restoration of blood perfusion. For the medical restoration of blood perfusion, the administered drugs are thrombolytics and fibrinolytics, which are used in a process called thrombolysis. Revascularization may be performed as a combination of surgical and medical restoration of blood perfusion to a body part or organ that has suffered ischemia.

The term “coronary revascularization” as used herein refers to the surgical or medical restoration of blood flow in blocked or stenosed coronary arteries. Surgeries performed may be minimally-invasive endovascular procedures such as a percutaneous coronary intervention (PCI), followed by a coronary angioplasty. The angioplasty uses the insertion of a balloon to open up the artery, with the possible additional placement of one or more stents. Other surgery performed is the more invasive coronary artery bypass surgery (CABG) that grafts arteries around coronary blockages or stenoses. For the medical restoration of blood perfusion, the administered drugs are thrombolytics and fibrinolytics, which are used in a process called thrombolysis. Coronary revascularization may be performed as a combination of surgical and medical restoration of blood flow in blocked coronary arteries.

The term “reperfusion” or “coronary reperfusion” as used herein refers to restoration of blood perfusion to a body part or organ that has suffered ischemia. Reperfusion may occur spontaneously or through revascularization, which may include medical or surgical treatment. “Coronary reperfusion” refers to restoration of blood flow in blocked or stenosed coronary arteries.

The term “myocardial ischemia/reperfusion” as used herein refers to restoration of blood perfusion to ischemic myocardium, such as for example the case of a myocardial ischemia that has reperfused.

The term “myocardial ischemia/reperfusion injury” as used herein refers to tissue damage caused when blood supply returns to myocardial tissue after a period of ischemia. The absence of oxygen and nutrients during the ischemic period creates a condition in which the restoration of circulation results in inflammation and oxidative damage through the induction of oxidative stress.

The term “sepsis” as used herein refers to a disease characterized by an infection that triggers an immune response leading to inflammation and organ dysfunction.

The term “sepsis-induced myocardial inflammation” as used herein refers to cellular or humoral inflammation in the myocardium associated with sepsis.

The term “septic cardiomyopathy” as used herein refers to cardiac dysfunction associated with sepsis.

The term “myocarditis” as used herein refers to a disease characterized by an inflammation of the myocardium induced by, but not limited to, viruses, bacteria, autoantibodies, activated leukocytes, toxins, drugs, or associated with systemic autoinflammatory diseases such as systemic lupus erythematosus, rheumatoid arthritis or sarcoidosis.

The term infectious myocarditis as used herein refers to myocarditis induced by an infectious agent.

The term autoimmune myocarditis as used herein refers to myocarditis induced by an autoimmune response against the myocardium or associated with an autoimmune disorder.

The term isolated myocarditis as used herein refers to myocarditis of unknown or undiagnosed cause.

As used herein, whenever a binding event between a compound of the disclosure and any one of S100A9, S100A8, the S100A8/A9 dimer, or the S100A8/A9 tetramer is described, such as for example inhibition or blocking of any one of S100A9, S100A8, the S100A8/A9 dimer, or the S100A8/A9 tetramer, it is to be understood that the binding event can be between the compound of the disclosure and any of S100A9, S100A8, the S100A8/A9 dimer, or the S100A8/A9 tetramer, even if the binding partner is not explicitly mentioned. For example, mention of a binding event between a compound of the disclosure and S100A9 is also taken to mean a binding event of the compound of the disclosure and S100A8, between a compound of the disclosure and the S100A8/A9 dimer, or between a compound of the disclosure and the S100A8/A9 tetramer.

### Compounds for Use

In one embodiment of the present disclosure, a compound of formula (I) is provided,

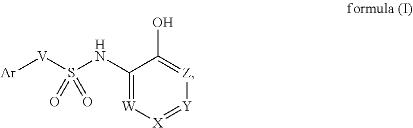

wherein

### W is CH or N;

### X is N or CR¹;

### Y is CR² or N;

### Z is CR³ or N;

at least one and at most two of W, X, Y, and Z are N;
R¹ is H, halogen, S(O)₂C1-C3 alkyl, cyano, or C1-C3 alkyl optionally substituted with one or more halogen(s);
R² is H, halogen, cyano, C(O)OH, C(O)OC1-C3 alkyl, C1-C3 alkyl optionally substituted with one or more F; hydroxy-C1-C3 alkyl, S(O)₂C1-C3 alkyl, S(O)₂C3-C6 cycloalkyl or S(O)₂C1-C3 hydroxyalkyl;
R³ is H, halogen or cyano;
V is (CHR⁴)ₘ;
m is 0 or 1;
R⁴ is H or C1-C3 alkyl optionally substituted with one or more halogen(s);

### Ar is

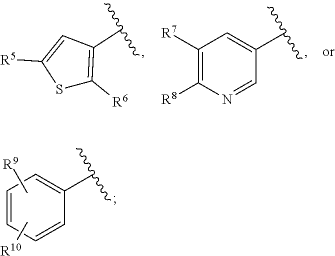

R⁵ is halogen, H, or cyano;
R⁶ is halogen, or H;
R⁷ is halogen, H, C1-C3 alkyl, cyano, S(O)₂C1-C3 alkyl, or phenyl;
R⁸ is C1-C3 alkoxy optionally substituted with one or more F, C1-C3 alkyl optionally substituted with one or more F, H, halogen, phenoxy, NHR¹¹, or NR¹¹R¹²;
R⁹ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹⁰ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹¹ is C1-C3 alkyl;
R¹² is C1-C3 alkyl; or
R¹¹ and R¹², together with the nitrogen atom to which they are both attached, form a ring of formula

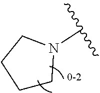

R¹³ is H or C1-C3 alkyl; and
R¹⁴ is H or C1-C3 alkyl;
or a pharmaceutically acceptable salt thereof, for use in the treatment of a disease or disorder involving myocardial inflammation.

In a further embodiment of the present disclosure, a compound of formula (I) is provided, wherein R⁹ is H or halogen and R¹⁰ is halogen or cyano, for use in the treatment of a disease or disorder involving myocardial inflammation.

In a further embodiment of the present disclosure, a compound of formula (I) is provided, wherein R⁵ and R⁶ are halogens, for use in the treatment of a disease or disorder involving myocardial inflammation.

In an embodiment of the present disclosure, a compound of formula (I) is provided, wherein Ar is

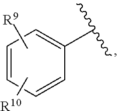

for use in the treatment of a disease or disorder involving myocardial inflammation.

In yet another embodiment of the present disclosure, a compound of formula (I) is provided, wherein Ar is

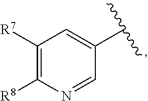

for use in the treatment of a disease or disorder involving myocardial inflammation.

In yet another embodiment of the disclosure, a compound of formula (I) is provided, wherein Ar is

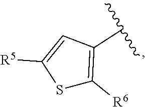

for use in the treatment of a disease or disorder involving myocardial inflammation.

In some embodiments of the present disclosure, a compound of formula (I) is provided, wherein Y is CR², for use in the treatment of a disease or disorder involving myocardial inflammation.

In one embodiment of the present disclosure, a compound of formula (I) is provided, wherein W is N, for use in the treatment of a disease or disorder involving myocardial inflammation. In another embodiment, X is N, for use in the treatment of a disease or disorder involving myocardial inflammation. In yet another embodiment, a compound of formula (I) is provided, wherein W and X are both N, for use in the treatment of a disease or disorder involving myocardial inflammation.

In one embodiment of the present disclosure, a compound of formula (I) is provided, wherein Y and Z are both CH, for use in the treatment of a disease or disorder involving myocardial inflammation. In another embodiment, a compound of formula (I) is provided, wherein Y is CH, Z is CR³, and R³ is halogen, for use in the treatment of a disease or disorder involving myocardial inflammation. In yet another embodiment, a compound of formula (I) is provided, wherein Y is CR², Z is CH, and R² is halogen or S(O)₂C1-C3 alkyl, for use in the treatment of a disease or disorder involving myocardial inflammation. In still another embodiment, a compound of formula (I) is provided, wherein Z is N, for use in the treatment of a disease or disorder involving myocardial inflammation.

In a preferred embodiment of the present disclosure, a compound of formula (I) is provided, wherein m is 0, for use in the treatment of a disease or disorder involving myocardial inflammation.

In an embodiment of the present disclosure, the compound for use as disclosed herein is selected from:
5-bromo-N-(5-chloro-4-hydroxypyridin-3-yl)-6-methoxypyridine-3-sulfonamide;
3,5-dichloro-N-(6-chloro-4-hydroxypyridazin-3-yl)benzene-1-sulfonamide;
2,5-dichloro-N-(4-hydroxypyridin-3-yl)thiophene-3-sulfonamide;
2,5-dichloro-N-(6-chloro-4-hydroxypyridin-3-yl)thiophene-3-sulfonamide;
2,5-dichloro-N-(5-chloro-2-hydroxypyridin-3-yl)thiophene-3-sulfonamide;
5-chloro-N-(5-chloro-4-hydroxypyridin-3-yl)-6-methylpyridine-3-sulfonamide;
5-bromo-6-chloro-N-(5-chloro-2-hydroxypyridin-3-yl)pyridine-3-sulfonamide;
N-(4-hydroxypyridin-3-yl)benzenesulfonamide;
N-(4-hydroxypyridin-3-yl)-4-(trifluoromethyl)benzene-1-sulfonamide;
N-(4-hydroxypyridin-3-yl)-4-(trifluoromethoxy)benzene-1-sulfonamide;
N-(5-chloro-3-hydroxypyridin-2-yl)-1-phenylmethanesulfonamide;
N-(5-chloro-3-hydroxypyridin-2-yl)-6-(pyrrolidin-1-yl)pyridine-3-sulfonamide;
N-(5-chloro-3-hydroxypyridin-2-yl)pyridine-3-sulfonamide;
6-chloro-N-(5-chloro-3-hydroxypyridin-2-yl)pyridine-3-sulfonamide;
N-(5-chloro-3-hydroxypyridin-2-yl)-1-(3,5-dichlorophenyl)methanesulfonamide;
N-(5-chloro-3-hydroxypyridin-2-yl)-6-[(propan-2-yl)amino]pyridine-3-sulfonamide;
5-bromo-6-chloro-N-[3-hydroxy-5-(propan-2-yl)pyridin-2-yl]pyridine-3-sulfonamide;
N-(5-chloro-3-hydroxypyridin-2-yl)-1-(3-cyanophenyl)methanesulfonamide;
(+)-5-bromo-6-chloro-N-[3-hydroxy-5-(1-hydroxypropan-2-yl)pyridin-2-yl]pyridine-3-sulfonamide;
(−)-5-bromo-6-chloro-N-[3-hydroxy-5-(1-hydroxypropan-2-yl)pyridin-2-yl]pyridine-3-sulfonamide;
5-bromo-6-chloro-N-(3-hydroxypyridin-2-yl)pyridine-3-sulfonamide;
N-(5-chloro-3-hydroxypyridin-2-yl)-1-(2,4-dichlorophenyl)methanesulfonamide;
N-(5-chloro-3-hydroxypyridin-2-yl)-1-(4-cyanophenyl)methanesulfonamide;
N-(5-chloro-3-hydroxypyridin-2-yl)-1-pyridin-3-ylmethanesulfonamide;
5-bromo-N-(5-chloro-3-hydroxypyridin-2-yl)-6-(pyrrolidin-1-yl)pyridine-3-sulfonamide;
N-(5-chloro-3-hydroxypyridin-2-yl)-1-(3,5-difluorophenyl)methanesulfonamide;
N-(5-chloro-3-hydroxypyridin-2-yl)-1-(2,5-dichlorothiophen-3-yl)methanesulfonamide;
N-(5-chloro-3-hydroxypyridin-2-yl)-1-(3,4-dichlorophenyl)methanesulfonamide;
N-(5-chloro-3-hydroxypyridin-2-yl)-1-(3-chloro-5-fluorophenyl)methanesulfonamide;
1-(2,4-dichlorophenyl)-N-(4-hydroxypyridin-3-yl)methanesulfonamide;
1-(3,5-dichlorophenyl)-N-(4-hydroxypyridin-3-yl)methanesulfonamide:
3,5-dichloro-N-(5-chloro-3-hydroxypyridin-2-yl)benzene-1-sulfonamide;
3,4-dichloro-N-(5-chloro-3-hydroxypyridin-2-yl)benzene-1-sulfonamide;
N-(5-chloro-3-hydroxypyridin-2-yl)-1-(3-chlorophenyl)methanesulfonamide;
N-(5-chloro-3-hydroxypyridin-2-yl)-1-(4-chlorophenyl)methanesulfonamide;
N-(5-chloro-3-hydroxypyridin-2-yl)-1-(2-chlorophenyl)methanesulfonamide;
N-(5-chloro-3-hydroxypyridin-2-yl)-1-(2,5-dichlorophenyl)methanesulfonamide;
N-(5-chloro-3-hydroxypyridin-2-yl)-1-(3,4-difluorophenyl)methanesulfonamide;
1-(3,5-dichlorophenyl)-N-(3-hydroxy-5-methanesulfonylpyridin-2-yl)methanesulfonamide;
N-(5-chloro-3-hydroxypyridin-2-yl)-1-(3-chloro-5-cyanophenyl)methanesulfonamide;
3-chloro-5-{[(5-chloro-3-hydroxypyridin-2-yl)sulfamoyl]methyl}benzamide;
1-(5-chloro-2-fluorophenyl)-N-(5-chloro-3-hydroxypyridin-2-yl)methanesulfonamide;
N-(6-chloro-4-hydroxypyridazin-3-yl)-1-(3,5-dichlorophenyl)methanesulfonamide;
N-(5-chloro-3-hydroxypyridin-2-yl)-1-(2,3-dichlorophenyl)methanesulfonamide;
N-(5-chloro-3-hydroxypyridin-2-yl)-1-(2,6-dichlorophenyl)methanesulfonamide;
(1R)-N-(5-chloro-3-hydroxypyridin-2-yl)-1-(3,5-dichlorophenyl)ethane-1-sulfonamide;
(1S)-N-(5-chloro-3-hydroxypyridin-2-yl)-1-(3,5-dichlorophenyl)ethane-1-sulfonamide;
5-bromo-N-(5-chloro-3-hydroxypyridin-2-yl)pyridine-3-sulfonamide;
N-(5-chloro-3-hydroxypyridin-2-yl)-6-(trifluoromethyl)pyridine-3-sulfonamide;
N-(5-chloro-3-hydroxypyridin-2-yl)-3-(trifluoromethoxy)benzene-1-sulfonamide;
N-(5-bromo-3-hydroxypyridin-2-yl)benzene sulfonamide;
N-(5-bromo-3-hydroxypyridin-2-yl)-2,5-dichlorothiophene-3-sulfonamide;
N-(5-bromo-3-hydroxypyridin-2-yl)-3-(trifluoromethoxy)benzene-1-sulfonamide;
5-bromo-N-(5-bromo-3-hydroxypyridin-2-yl)-6-chloropyridine-3-sulfonamide;
N-(5-bromo-3-hydroxypyridin-2-yl)-6-(trifluoromethyl)pyridine-3-sulfonamide;
5-bromo-N-(5-bromo-3-hydroxypyridin-2-yl)-6-methoxypyridine-3-sulfonamide;
N-(3-hydroxypyridin-2-yl)-6-(trifluoromethyl)pyridine-3-sulfonamide;
methyl 6-(2,5-dichlorothiophene-3-sulfonamido)-5-hydroxypyridine-3-carboxylate;
methyl 6-benzenesulfonamido-5-hydroxypyridine-3-carboxylate;
4-bromo-3-fluoro-N-(4-hydroxypyridin-3-yl)benzene-1-sulfonamide;
N-(5-chloro-2-hydroxypyridin-3-yl)-6-(trifluoromethyl)pyridine-3-sulfonamide;
5-bromo-N-(5-chloro-2-hydroxypyridin-3-yl)-6-methoxypyridine-3-sulfonamide;
2,5-dichloro-N-(5-chloro-3-hydroxypyridin-2-yl)thiophene-3-sulfonamide;
N-(5-chloro-4-hydroxypyridin-3-yl)-6-(trifluoromethyl)pyridine-3-sulfonamide;
5-bromo-6-chloro-N-(5-chloro-4-hydroxypyridin-3-yl)pyridine-3-sulfonamide;
5-bromo-N-(5-chloro-4-hydroxypyridin-3-yl)pyridine-3-sulfonamide;
5-bromo-6-chloro-N-(4-hydroxypyridin-3-yl)pyridine-3-sulfonamide;
5-bromo-N-(4-hydroxypyridin-3-yl)-6-methoxypyridine-3-sulfonamide;
N-(4-hydroxypyridin-3-yl)-6-(trifluoromethyl)pyridine-3-sulfonamide;
3,4-difluoro-N-(4-hydroxypyridin-3-yl)benzene-1-sulfonamide;
3,4-dichloro-N-(4-hydroxypyridin-3-yl)benzene-1-sulfonamide;
N-(2-hydroxypyridin-3-yl)-6-(trifluoromethyl)pyridine-3-sulfonamide;
5-bromo-6-chloro-N-(2-hydroxypyridin-3-yl)pyridine-3-sulfonamide;
2,5-dichloro-N-(2-hydroxypyridin-3-yl)thiophene-3-sulfonamide;
N-(6-chloro-4-hydroxypyridin-3-yl)-1-(3,4-dichlorophenyl)methanesulfonamide;
5-bromo-N-(5-chloro-3-hydroxypyridin-2-yl)-6-methoxypyridine-3-sulfonamide;
3,4-dichloro-N-(3-hydroxypyridin-4-yl)benzene-1-sulfonamide;
N-(5-bromo-3-hydroxypyrazin-2-yl)-1-(3,4-dichlorophenyl)methanesulfonamide;
N-(6-chloro-4-hydroxypyridazin-3-yl)-1-(3,4-difluorophenyl)methanesulfonamide;
1-(3,5-dichlorophenyl)-N-[3-hydroxy-5-(propane-2-sulfonyl)pyridin-2-yl]methanesulfonamide;
N-(5-chloro-3-hydroxypyrazin-2-yl)-1-(3,5-dichlorophenyl)methanesulfonamide;
5-bromo-N-(5-chloro-4-hydroxypyridin-3-yl)-6-phenoxypyridine-3-sulfonamide;
N-(5-bromo-3-hydroxypyrazin-2-yl)-1-(3,5-dichlorophenyl)methanesulfonamide;
N-(6-chloro-4-hydroxypyridazin-3-yl)-1-(2,4-dichlorophenyl)methanesulfonamide;
1-(3,5-dichlorophenyl)-N-(4-hydroxy-6-iodopyridazin-3-yl)methanesulfonamide;
N-(6-bromo-4-hydroxypyridazin-3-yl)-1-(3,5-dichlorophenyl)methanesulfonamide;
3-bromo-N-(5-bromo-4-hydroxypyridin-3-yl)-4-methoxybenzene-1-sulfonamide;
N-(6-chloro-4-hydroxypyridazin-3-yl)-1-(2-chloro-5-cyanophenyl)methane-sulfonamide;
N-(5-chloro-4-hydroxypyridin-3-yl)-6-phenoxypyridine-3-sulfonamide;
N-(6-chloro-4-hydroxypyridin-3-yl)-1-(3,5-dichlorophenyl)methanesulfonamide;
1-(3-chlorophenyl)-N-[5-(ethanesulfonyl)-3-hydroxypyrazin-2-yl]methane-sulfonamide;
3,5-dichloro-N-(5-chloro-4-hydroxypyridin-3-yl)benzene-1-sulfonamide;
N-(6-chloro-4-hydroxypyridazin-3-yl)-1-(2-chlorophenyl)methanesulfonamide;
5-bromo-N-(5-bromo-4-hydroxypyridin-3-yl)-6-methoxypyridine-3-sulfonamide;
N-(6-bromo-5-chloro-3-hydroxypyridin-2-yl)-1-(3,5-dichlorophenyl)methanesulfonamide
1-(2-chlorophenyl)-N-(4-hydroxy-6-methanesulfonylpyridazin-3-yl)methanesulfonamide
N-(6-chloro-4-hydroxypyridazin-3-yl)-1-(3-chloro-5-fluorophenyl)methane-sulfonamide;
N-(6-chloro-4-hydroxypyridazin-3-yl)-1-(3-chlorophenyl)methanesulfonamide;
N-(5-bromo-4-hydroxypyridin-3-yl)-1-(3,5-dichlorophenyl)methanesulfonamide;
3-[(5-chloro-4-hydroxypyridin-3-yl)sulfamoyl]-N,N-diethylbenzamide;
1-(3,4-difluorophenyl)-N-(4-hydroxy-6-methanesulfonylpyridazin-3-yl)methanesulfonamide;
3-chloro-N-(5-chloro-4-hydroxypyridin-3-yl)-4-methylbenzene-1-sulfonamide;
5-bromo-N-(5-chloro-4-hydroxypyridin-3-yl)-6-(propan-2-yloxy)pyridine-3-sulfonamide;
3-chloro-N-(5-chloro-4-hydroxypyridin-3-yl)-4-(trifluoromethoxy)benzene-1-sulfonamide
N-(5-cyano-3-hydroxypyrazin-2-yl)-1-(3,5-dichlorophenyl)methanesulfonamide;
1-(3,5-dichlorophenyl)-N-[4-hydroxy-6-(propane-1-sulfonyl)pyridazin-3-yl]methane-sulfonamide;
N-(5-chloro-3-hydroxypyridin-2-yl)-1-(3,5-dimethoxyphenyl)methanesulfonamide;
5-chloro-N-(5-chloro-3-hydroxypyridin-2-yl)-6-(dimethylamino)pyridine-3-sulfonamide;
N-(2-chloro-4-hydroxypyrimidin-5-yl)-1-(3,5-dichlorophenyl)methanesulfonamide;
1-(3,5-dichlorophenyl)-N-[5-(ethanesulfonyl)-3-hydroxypyridin-2-yl]methane-sulfonamide;
3,4-dichloro-N-(6-chloro-4-hydroxypyridazin-3-yl)benzene-1-sulfonamide;
N-(5-chloro-4-hydroxypyridin-3-yl)-1-(3,5-dichlorophenyl)methanesulfonamide;
1-(3,5-dichlorophenyl)-N-[6-(ethanesulfonyl)-4-hydroxypyridazin-3-yl]methanesulfonamide;
1-(3,5-dichlorophenyl)-N-(3-hydroxy-5-methanesulfonylpyrazin-2-yl)methane-sulfonamide;
2,5-dichloro-N-(6-chloro-4-hydroxypyridazin-3-yl)thiophene-3-sulfonamide;
5-bromo-N-(6-chloro-4-hydroxypyridazin-3-yl)-6-methoxypyridine-3-sulfonamide;
N-(6-chloro-4-hydroxypyridazin-3-yl)-1-(2,3-dichlorophenyl)methanesulfonamide;
N-(6-chloro-4-hydroxypyridazin-3-yl)-3-(trifluoromethoxy)benzene-1-sulfonamide;
6-(azetidin-1-yl)-5-bromo-N-(5-chloro-4-hydroxypyridin-3-yl)pyridine-3-sulfonamide;
1-(3,5-dichlorophenyl)-N-[4-hydroxy-6-(propane-2-sulfonyl)pyridazin-3-yl]methanesulfonamide;
5-bromo-N-(5-chloro-4-hydroxypyridin-3-yl)-6-ethoxypyridine-3-sulfonamide;
5-bromo-N-(5-chloro-3-hydroxypyridin-2-yl)-6-(dimethylamino)pyridine-3-sulfonamide;
N-(6-chloro-4-hydroxypyridazin-3-yl)-3-cyanobenzene-1-sulfonamide;
3-chloro-N-(5-chloro-4-hydroxypyridin-3-yl)-4-methoxybenzene-1-sulfonamide;
3-bromo-N-(5-chloro-4-hydroxypyridin-3-yl)-4-methoxybenzene-1-sulfonamide;
1-(3,5-dichlorophenyl)-N-[3-hydroxy-5-(propane-1-sulfonyl)pyridin-2-yl]methanesulfonamide;
N-(6-chloro-4-hydroxypyridazin-3-yl)-1-(3,4-dichlorophenyl)methanesulfonamide;
1-(3,5-dichlorophenyl)-N-(4-hydroxy-6-methanesulfonylpyridazin-3-yl)methanesulfonamide;
N-(5-chloro-3-hydroxypyridin-2-yl)-1-(5-cyanothiophen-3-yl)methanesulfonamide;
5-bromo-6-chloro-N-[4-hydroxy-6-(trifluoromethyl)pyridin-3-yl]pyridine-3-sulfonamide;
5-bromo-N-(5-chloro-4-hydroxypyridin-3-yl)-6-(piperidin-1-yl)pyridine-3-sulfonamide;
N-(5-chloro-6-cyano-3-hydroxypyridin-2-yl)-1-(3,5-dichlorophenyl)methanesulfonamide;
N-[5-(cyclopentanesulfonyl)-3-hydroxypyridin-2-yl]-1-(3,5-dichlorophenyl)methanesulfonamide;
N-[4-hydroxy-6-(trifluoromethyl)pyridin-3-yl]-6-methylpyridine-3-sulfonamide;
1-(3,5-dichlorophenyl)-N-[4-hydroxy-6-(trifluoromethyl)pyridin-3-yl]methanesulfonamide;
N-(5-chloro-3-hydroxy-6-methanesulfonylpyridin-2-yl)-1-(3,5-dichlorophenyl)methanesulfonamide;
1-(3,4-dichlorophenyl)-N-[4-hydroxy-6-(trifluoromethyl)pyridin-3-yl]methanesulfonamide;
N-(5-chloro-3-hydroxypyridin-2-yl)-6-(dimethylamino)-5-methanesulfonylpyridine-3-sulfonamide;
5-bromo-N-[4-hydroxy-6-(trifluoromethyl)pyridin-3-yl]-6-methoxypyridine-3-sulfonamide;
N-(5-cyano-4-hydroxypyridin-3-yl)-1-(3,5-dichlorophenyl)methanesulfonamide;
1-(3,5-dichlorophenyl)-N-[4-hydroxy-6-(3-hydroxypropanesulfonyl)pyridazin-3-yl]methanesulfonamide;
N-[6-(cyclopentanesulfonyl)-4-hydroxypyridazin-3-yl]-1-(3,5-dichlorophenyl)methane-sulfonamide;
(+/−)-N-(5-bromo-3-hydroxypyrazin-2-yl)-1-(3,5-dichlorophenyl)-2,2,2-trifluoro-ethane-1-sulfonamide;
3-[(5-bromo-3-hydroxypyrazin-2-yl)sulfamoyl]-N,N-diethylbenzamide;
N-(2-chloro-5-hydroxypyrimidin-4-yl)-1-(3,5-dichlorophenyl)methanesulfonamide;
3,4-dichloro-N-(2-chloro-5-hydroxypyrimidin-4-yl)benzene-1-sulfonamide;
3,5-dichloro-N-(2-chloro-5-hydroxypyrimidin-4-yl)benzene-1-sulfonamide;
3-[(6-chloro-4-hydroxypyridazin-3-yl)sulfamoyl]-N, N-diethylbenzamide;
N-(6-chloro-4-hydroxypyridazin-3-yl)-1-(4-cyanophenyl)methanesulfonamide;
N-(6-chloro-4-hydroxypyridazin-3-yl)-1-(5-cyano-2-fluorophenyl)methane-sulfonamide;
N-(6-chloro-4-hydroxypyridazin-3-yl)-1-(3-cyano-5-fluorophenyl)methane-sulfonamide;
1-(2-chloro-5-cyanophenyl)-N-(2-chloro-5-hydroxypyrimidin-4-yl)methane-sulfonamide;
2-chloro-N-(6-chloro-4-hydroxypyridazin-3-yl)-4-cyanobenzene-1-sulfonamide;
3-chloro-N-(6-chloro-4-hydroxypyridazin-3-yl)-4-fluorobenzene-1-sulfonamide;
3,5-dichloro-N-(5-cyano-4-hydroxypyridin-3-yl)benzene-1-sulfonamide;
3-chloro-N-(6-chloro-4-hydroxypyridazin-3-yl)-5-fluorobenzene-1-sulfonamide;
3,5-dichloro-N-(4-hydroxy-6-methanesulfonylpyridin-3-yl)benzene-1-sulfonamide;
3,5-dichloro-N-(6-chloro-4-hydroxypyridin-3-yl)benzene-1-sulfonamide;
1-(3,5-dichlorophenyl)-N-[5-hydroxy-2-(trifluoromethyl)pyrimidin-4-yl]methanesulfonamide;
3-chloro-5-fluoro-N-[5-hydroxy-2-(trifluoromethyl)pyrimidin-4-yl]benzene-1-sulfonamide;
3,5-dichloro-N-(3-hydroxy-5-methanesulfonylpyrazin-2-yl)benzene-1-sulfonamide;
3-chloro-4-[(6-chloro-4-hydroxypyridazin-3-yl)sulfamoyl]-N,N-diethylbenzamide;
3-chloro-5-{[(6-chloro-4-hydroxypyridazin-3-yl)sulfamoyl]methyl}-N,N-diethylbenzamide
N-(6-chloro-4-hydroxypyridazin-3-yl)-3-cyano-5-fluorobenzene-1-sulfonamide;
3-chloro-5-fluoro-N-[4-hydroxy-6-(trifluoromethyl)pyridazin-3-yl]benzene-1-sulfonamide;
1-(3,4-dichlorophenyl)-N-[5-hydroxy-2-(trifluoromethyl)pyrimidin-4-yl]methanesulfonamide;
N-(6-chloro-4-hydroxypyridazin-3-yl)-1-(2,5-dichlorothiophen-3-yl)methanesulfonamide;
3,5-dichloro-N-(4-hydroxy-6-methanesulfonylpyridazin-3-yl)benzene-1-sulfonamide;
1-(3,5-dichlorophenyl)-N-(3-hydroxypyridin-4-yl)methanesulfonamide;
6-(2,5-dichlorothiophene-3-sulfonamido)-5-hydroxypyridine-3-carboxylic acid;
N-(2-chloro-3-hydroxypyridin-4-yl)-1-(3,5-dichlorophenyl)methanesulfonamide;
3,5-dichloro-N-(2-chloro-3-hydroxypyridin-4-yl)benzene-1-sulfonamide;
N-(3-hydroxypyridin-2-yl)-3-(trifluoromethyl)benzene-1-sulfonamide;
N-(5-chloro-2-hydroxypyridin-3-yl)-1-(3,4-dichlorophenyl)methanesulfonamide;
N-(5,6-dichloro-2-hydroxypyridin-3-yl)-1-(3,5-dichlorophenyl)methanesulfonamide;
N-(6-chloro-2-hydroxypyridin-3-yl)-1-(3,4-dichlorophenyl)methanesulfonamide;
N-(6-chloro-2-hydroxypyridin-3-yl)-1-(3,5-dichlorophenyl)methanesulfonamide;
5-bromo-N-(6-chloro-2-hydroxypyridin-3-yl)-6-methoxypyridine-3-sulfonamide;
N-(5,6-dibromo-3-hydroxypyrazin-2-yl)-1-(3,4-dichlorophenyl)methanesulfonamide;
N-(6-chloro-2-hydroxypyridin-3-yl)-1-[4-(trifluoromethyl)phenyl]methane-sulfonamide;
N-(6-chloro-2-hydroxypyridin-3-yl)-4-propylbenzene-1-sulfonamide;
3,4-dichloro-N-(6-chloro-2-hydroxypyridin-3-yl)benzene-1-sulfonamide;
N-(6-chloro-2-hydroxypyridin-3-yl)-1-(5,6-dichloropyridin-3-yl)methane-sulfonamide;
N-(6-chloro-2-hydroxypyridin-3-yl)-1-(5-chloro-6-methoxypyridin-3-yl)methanesulfonamide;
N-(5-chloro-4-hydroxypyridin-3-yl)-1-(5,6-dichloropyridin-3-yl)methane-sulfonamide;
N-(5-chloro-4-hydroxypyridin-3-yl)-1-(5-chloro-6-methoxypyridin-3-yl)methanesulfonamide;
N-(6-chloro-2-hydroxypyridin-3-yl)-1-[5-chloro-6-(pyrrolidin-1-yl)pyridin-3-yl]methanesulfonamide;
N-(6-chloro-2-hydroxypyridin-3-yl)-1-[3-chloro-5-(ethylsulfanyl)phenyl]methane-sulfonamide;
3,5-dichloro-N-[6-(ethanesulfonyl)-2-hydroxypyridin-3-yl]benzene-1-sulfonamide;
1-(3,5-dichlorophenyl)-N-[6-(ethanesulfonyl)-2-hydroxypyridin-3-yl]methanesulfonamide;
N-(5-cyano-3-hydroxypyridin-2-yl)-1-(3,5-dichlorophenyl)methanesulfonamide;
N-(5-chloro-4-hydroxypyridin-3-yl)-5-cyano-6-methoxypyridine-3-sulfonamide;
N-(5-chloro-4-hydroxypyridin-3-yl)-6-methoxy-5-phenylpyridine-3-sulfonamide;
N-(5-chloro-3-hydroxypyridin-2-yl)-5-phenylpyridine-3-sulfonamide;
N-(6-chloro-4-hydroxypyridazin-3-yl)-1-(3-chloro-5-cyanophenyl)methane sulfonamide
5-bromo-6-chloro-N-(6-chloro-4-hydroxypyridazin-3-yl)pyridine-3-sulfonamide; and
N-(5-bromo-4-hydroxypyridin-3-yl)-3,5-dichlorobenzene-1-sulfonamide;
   or a pharmaceutically acceptable salt thereof.

In one embodiment, a compound according to formula (I) is provided, wherein

### W is N or CH;

### X is N or CR¹;

### Y is CR²

### Z is N or CR³;

at least one and at most two of W, X, Y, and Z are N;
R¹ is halogen;
R² is H or halogen;
R³ is H or halogen;
V is (CHR⁴)ₘ;
m is 0;
R⁴ is H or C1-C3 alkyl optionally substituted with one or more halogen(s);

### Ar is

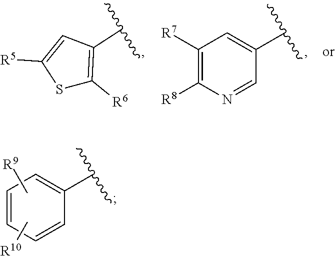

R⁵ is halogen;
R⁶ is halogen;
R⁷ is halogen;
R³ is C1-C3 alkoxy or C1-C3 alkyl;
R⁹ is halogen; and
R¹⁰ is halogen,
or a pharmaceutically acceptable salt thereof, for use in the treatment of a disease or disorder involving myocardial inflammation.

In a preferred embodiment, the compound for use as disclosed herein is selected from the group consisting of:
5-bromo-N-(5-chloro-4-hydroxypyridin-3-yl)-6-methoxypyridine-3-sulfonamide;
3,5-dichloro-N-(6-chloro-4-hydroxypyridazin-3-yl)benzene-1-sulfonamide;
2,5-dichloro-N-(4-hydroxypyridin-3-yl)thiophene-3-sulfonamide;
2,5-dichloro-N-(6-chloro-4-hydroxypyridin-3-yl)thiophene-3-sulfonamide;
2,5-dichloro-N-(5-chloro-2-hydroxypyridin-3-yl)thiophene-3-sulfonamide; and
5-chloro-N-(5-chloro-4-hydroxypyridin-3-yl)-6-methylpyridine-3-sulfonamide
   or a pharmaceutically acceptable salt thereof.

In a more preferred embodiment, the compound for use as disclosed herein is 5-bromo-N-(5-chloro-4-hydroxypyridin-3-yl)-6-methoxypyridine-3-sulfonamide or a pharmaceutically acceptable salt thereof.

In another preferred embodiment, the compound the compound for use as disclosed herein is 3,5-dichloro-N-(6-chloro-4-hydroxypyridazin-3-yl)benzene-1-sulfonamide or a pharmaceutically acceptable salt thereof.

In yet another preferred embodiment, the compound the compound for use as disclosed herein is 2,5-dichloro-N-(4-hydroxypyridin-3-yl)thiophene-3-sulfonamide or a pharmaceutically acceptable salt thereof.

In yet another preferred embodiment, the compound the compound for use as disclosed herein is 2,5-dichloro-N-(6-chloro-4-hydroxypyridin-3-yl)thiophene-3-sulfonamide or a pharmaceutically acceptable salt thereof.

In yet another preferred embodiment, the compound the compound for use as disclosed herein is 2,5-dichloro-N-(6-chloro-4-hydroxypyridin-3-yl)thiophene-3-sulfonamide or a pharmaceutically acceptable salt thereof.

In still another more preferred embodiment, the compound the compound for use as disclosed herein is 5-chloro-N-(5-chloro-4-hydroxypyridin-3-yl)-6-methylpyridine-3-sulfonamide or a pharmaceutically acceptable salt thereof.

In one embodiment, a method of treating a disease or disorder involving myocardial inflammation is provided, said method comprising administering a therapeutically effective amount of a compound according to formula (I)

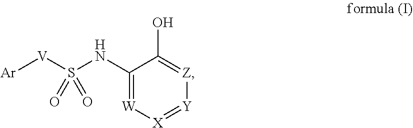

wherein

### W is CH or N;

### X is N or CR¹;

### Y is CR² or N;

### Z is CR³ or N;

at least one and at most two of W, X, Y, and Z are N;
R¹ is H, halogen, S(O)₂C1-C3 alkyl, cyano, or C1-C3 alkyl optionally substituted with one or more halogen(s);
R² is H, halogen, cyano, C(O)OH, C(O)OC1-C3 alkyl, C1-C3 alkyl optionally substituted with one or more F; hydroxy-C1-C3 alkyl, S(O)₂C1-C3 alkyl, S(O)₂C3-C6 cycloalkyl or S(O)₂C1-C3 hydroxyalkyl;
R³ is H, halogen or cyano;
V is (CHR⁴)ₘ;
m is 0 or 1;
R⁴ is H or C1-C3 alkyl optionally substituted with one or more halogen(s);

### Ar is

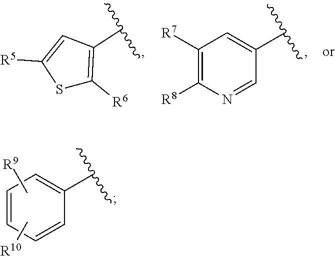

R⁵ is halogen, H, or cyano;
R⁶ is halogen, or H;
R⁷ is halogen, H, C1-C3 alkyl, cyano, S(O)₂C1-C3 alkyl, or phenyl;
R⁸ is C1-C3 alkoxy optionally substituted with one or more F, C1-C3 alkyl optionally substituted with one or more F, H, halogen, phenoxy, NHR¹¹, or NR¹¹R¹²;
R⁹ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹⁰ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹¹ is C1-C3 alkyl;
R¹² is C1-C3 alkyl; or
R¹¹ and R¹², together with the nitrogen atom to which they are both attached, form a ring of formula

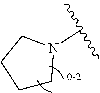

R¹³ is H or C1-C3 alkyl; and
R¹⁴ is H or C1-C3 alkyl;
or a pharmaceutically acceptable salt thereof to a subject in need thereof.

In one embodiment, the present disclosure relates to use of a compound according to formula (I)

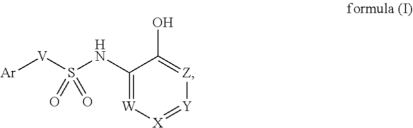

wherein

### W is CH or N;

### X is N or CR¹;

### Y is CR² or N;

### Z is CR³ or N;

at least one and at most two of W, X, Y, and Z are N;
R¹ is H, halogen, S(O)₂C1-C3 alkyl, cyano, or C1-C3 alkyl optionally substituted with one or more halogen(s);
R² is H, halogen, cyano, C(O)OH, C(O)OC1-C3 alkyl, C1-C3 alkyl optionally substituted with one or more F; hydroxy-C1-C3 alkyl, S(O)₂C1-C3 alkyl, S(O)₂C3-C6 cycloalkyl or S(O)₂C1-C3 hydroxyalkyl;
R³ is H, halogen or cyano;
V is (CHR⁴)ₘ;
m is 0 or 1;
R⁴ is H or C1-C3 alkyl optionally substituted with one or more halogen(s);

### Ar is

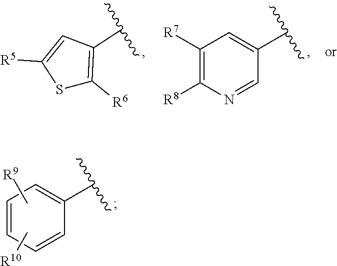

R⁵ is halogen, H, or cyano;
R⁶ is halogen, or H;
R⁷ is halogen, H, C1-C3 alkyl, cyano, S(O)₂C1-C3 alkyl, or phenyl;
R⁸ is C1-C3 alkoxy optionally substituted with one or more F, C1-C3 alkyl optionally substituted with one or more F, H, halogen, phenoxy, NHR¹¹, or NR¹¹R¹²;
R⁹ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹⁰ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹¹ is C1-C3 alkyl;
R¹² is C1-C3 alkyl; or
R¹¹ and R¹², together with the nitrogen atom to which they are both attached, form a ring of formula

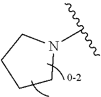

R¹³ is H or C1-C3 alkyl; and
R¹⁴ is H or C1-C3 alkyl;
or a pharmaceutically acceptable salt thereof for the manufacture of a medicament for the treatment of a disease or disorder involving a myocardial inflammation.

### S100A8/A9 Inhibition

Compounds of the present disclosure have been shown to interact with the S100A9 protein and to inhibit the activity of said protein. S100A9 and its dimerization partner S100A8 are pro-inflammatory alarmins that are produced and stored in large amounts in neutrophils. During the acute phase of the MI, sepsis and myocarditis, S100A8/A9 is rapidly released extracellularly from damaged tissues and activated immune cells, and amplifies the inflammatory response by interacting with its receptors advanced glycation end products (RAGE), toll-like receptor 4 (TLR4), and the extracellular matrix metalloproteinase inducer (EMMPRIN), located on the cell surface.

Thus, in one embodiment, the compound for use according to the present disclosure associates with the S100A9 protein, the S100A8 protein, the S100A8/A9 dimer and/or the S100A8/A9 tetramer.

In one embodiment, association of the compound for use of the present disclosure with the S100A9 protein, the S100A8 protein, the S100A8/A9 dimer and/or the S100A8/A9 tetramer result in inhibition of the association partner.

Inhibition of the extracellular S100A9 protein, the S100A8 protein, the S100A8/A9 dimer and/or the S100A8/A9 tetramer may result in reduced activation of the cell-surface receptors RAGE, TLR4, and EMMPRIN, which may lead to reduced inflammation.

Thus, in one embodiment, the present invention relates to a method of treating a disease or disorder involving myocardial inflammation, said method comprising administering an S100A9 inhibitor.

### Acute Inflammation in the Myocardium

The present disclosure relates to treatment of a disease or disorder involving myocardial inflammation.

Myocardial inflammation may result from diseases or disorders such as myocardial infarction due to myocardial ischemia or myocardial ischemia/reperfusion; myocarditis; or sepsis. Thus, in one embodiment, the disease or disorder is selected from the group consisting of myocardial infarction due to myocardial ischemia, myocardial infarction due to myocardial ischemia/reperfusion; myocarditis; sepsis; sepsis-induced myocardial inflammation; or septic cardiomyopathy.

Myocardial ischemia or myocardial ischemia/reperfusion leading to ischemia/reperfusion injury may result from totally or partially reduced blood flow to the myocardium, in some cases followed by reperfusion. Myocardial ischemia or myocardial ischemia/reperfusion can lead to myocardial damage and to myocardial infarction. Thus, in one embodiment, the disease or disorder of the present disclosure is myocardial infarction.

In one embodiment, the disease or disorder is an acute ischemic heart disease (ICD-10, I21-I24), such as for example acute myocardial infarction (ICD-10, I21), subsequent myocardial infarction (ICD-10, I22), other forms of acute ischemic heart disease (ICD-10, I24.8) or acute ischemic heart disease, unspecified (ICD-10, I24.9).

In one embodiment, the acute myocardial infarction (ICD-10, I21) is selected from acute transmural myocardial infarction (STEMI equivalent) (ICD-10, I21.0-21.3), acute subendocardial myocardial infarction (NSTEMI equivalent) (ICD-10, I21.4), or acute myocardial infarction, unspecified (ICD-10, I21.9).

In one embodiment, the acute myocardial infarction is acute transmural myocardial infarction (STEMI).

In one embodiment, the acute myocardial infarction is acute subendocardial myocardial infarction (NSTEMI).

In one embodiment, the acute myocardial infarction is acute myocardial infarction, unspecified.

In one embodiment, the other forms of acute ischemic heart disease (ICD-10, I24.8) is selected from coronary failure or coronary insufficiency.

In one embodiment, the MI is selected from type 1, type 2, type 4 or type 5 MI.

In one embodiment, the MI is selected from type 1 or type 2 MI.

In one embodiment, the MI is caused by a total or a sub-total coronary artery occlusion.

Thus, in one embodiment, the MI is a type 1 MI.

In one embodiment, the myocardial infarction is selected from the group consisting of ST-segment elevation myocardial infarction (STEMI) and non-ST-segment elevation myocardial infarction (NSTEMI).

In one embodiment, the myocardial infarction is ST-segment elevation myocardial infarction (STEMI). Depending on the treatment timing and severity of STEMI, it may result in a subendocardial or a transmural myocardial infarction.

In one embodiment, the myocardial infarction is non-ST-segment elevation myocardial infarction (NSTEMI). NSTEMI usually results in a subendocardial myocardial infarction.

Myocarditis is inflammation of the myocardium induced by, but not limited to, virus infections, bacterial infections, medications, toxins, and autoimmune disorders. Myocardial inflammation may lead to myocardial injury, cardiac dysfunction and cardiac dilation. Serum S100A8/A9 has been found to be increased in patients with myocarditis, and closely reflects the degree of myocardial inflammation and dysfunction. Thus, S100A8/A9 might be involved in the pathogenesis of myocarditis. Blockade of S100A9, S100A8, S100A8/A9 or of the S100A8/A9 tetramer with the compounds described in the invention reduce myocardial inflammation and prevent cardiac dysfunction in myocarditis.

Accordingly, in one embodiment, the disease or disorder is myocarditis.

In one embodiment, the disease or disorder is acute myocarditis (ICD-10, I40), such as for example infective myocarditis (ICD-10, I40.0), isolated myocarditis (ICD-10, I40.1), other acute myocarditis (ICD-10, I40.8), or acute myocarditis, unspecified (ICD-10, I40.9).

In one embodiment, the acute myocarditis is selected from the group consisting of infective myocarditis, autoimmune myocarditis and isolated myocarditis.

In one embodiment, the acute myocarditis is infective myocarditis.

In one embodiment, the acute myocarditis is autoimmune myocarditis.

In one embodiment, the acute myocarditis is isolated myocarditis.

In one embodiment, the disease or disorder is myocarditis in diseases classified elsewhere (ICD-10, I41), such as for example bacterial (ICD-10, I41.0), viral (ICD-10, I41.1), other infectious and parasitic diseases classified elsewhere (ICD-10, I41.2) or myocarditis in other diseases classified elsewhere (ICD-10, I41.8), such as rheumatoid myocarditis or sarcoid myocarditis.

During sepsis, the myocardium might become inflamed due to immune cell infiltration or to infiltration of pro-inflammatory mediators such as alarmins, pro-inflammatory cytokines, bacterial products, and others. Also, a direct inhibitory effect on cardiomyocyte activity by such mediators may lead to septic cardiomyopathy. S100A8/A9 is highly increased in the blood of sepsis patients, and may mediate both sepsis-induced myocardial inflammation and sepsis-induced cardiomyopathy. Blockade of S100A8/A9 with the compounds of the invention inhibits cardiac inflammation and improves sepsis-induced cardiomyopathy.

Accordingly, in one embodiment of the disclosure, the disease or disorder is sepsis.

In one embodiment, the disease or disorder is septic cardiomyopathy or sepsis-induced myocardial inflammation.

In one embodiment, the disease or disorder is septic cardiomyopathy.

In one embodiment, the disease or disorder is sepsis-induced myocardial inflammation.

### Treatment

The present disclosure relates to treatment of a disease or disorder involving myocardial inflammation.

Thus, in one embodiment, the compound as disclosed herein is administered to a subject in need thereof to provide treatment of a disease or disorder involving myocardial inflammation.

In one embodiment of the present disclosure, the compound as disclosed herein is administered to a patient in need thereof, together with at least one pharmaceutically acceptable excipient.

Administration of the compound as disclosed herein may be combined with coronary revascularization. In a preferred embodiment, a patient suffering from inflammation of the myocardium is treated by a combination of revascularisation and administration of the compound as disclosed herein. Thus, in one embodiment, treatment by administration of the compound as disclosed herein is performed in combination with coronary revascularization.

In a preferred embodiment, a patient suffering from myocardial ischemia is treated by a combination of revascularisation and administration of the compound as disclosed herein.

In one embodiment, treatment by administration of the compound as disclosed herein is not combined with coronary revascularization.

In a preferred embodiment, a patient suffering from acute myocardial infarction is treated by a combination of revascularisation and administration of the compound as disclosed herein.

In one embodiment, the administration of the compound as disclosed herein is performed during the acute phase of the disease or disorder.

In one embodiment, the coronary revascularization and administration of the compound as disclosed herein takes place during the acute phase of the disease or disorder.

In one embodiment, the administration of the compound of the present disclosure is discontinued after the acute phase of the disorder, such as discontinued before onset of the reparatory phase of the disease or disorder.

In one embodiment, the compound as described herein is administered during the acute phase of the disease or disorder, such as acute myocardial infarction, while the administration is discontinued before onset of the reparatory phase of the disease or disorder, e.g. the myocardial infarction.

In one embodiment of the disclosure, administration of the compound of the present disclosure to a subject is initiated once the level of S100A8/A9 measured in the blood of the subject is elevated compared to the normal concentration range of S100A8/A9 in the blood of healthy subjects.

In another embodiment of the disclosure, administration of the compound of the present disclosure is continued for as long as the level of S100A8/A9 in the blood of the subject is elevated compared to the normal concentration range of S100A8/A9 in the blood of healthy subjects.

In one embodiment of the disclosure, administration of the compound of the present disclosure is no longer continued once a normal level of S100A8/A9 is found in the blood of the subject.

In a specific embodiment of the disclosure, administration of the compound of the present disclosure is initiated once the level of S100A8/A9 measured in the blood of the subject is elevated compared to the normal concentration range of S100A8/A9 in the blood of healthy subjects, and administration is continued until the level of S100A8/A9 is within the normal concentration range of S100A8/A9 in the blood of healthy subjects.

In one embodiment, the compound is administered at least once daily, such as at least twice daily, such as at least three times daily. In one embodiment, the compound is administered twice daily. In a preferred embodiment, the compound is administered once daily.

In one embodiment of the present disclosure, the compound as disclosed herein is administered to a patient in need thereof for one, two, three, four, or five days after the onset of the disease or disorder. In a preferred embodiment, the compound as disclosed herein is administered to the patient for one, two, or three days after the onset of the disease or disorder. In one embodiment, the compound as disclosed herein is administered to the patient for three days after the onset of the disease or disorder. In one embodiment, the compound as disclosed herein is administered to the patient for two days after the onset of the disease or disorder. In one embodiment, the compound as disclosed herein is administered to the patient for one day after the onset of the disease or disorder.

In one embodiment of the present disclosure, the compound as disclosed herein is administered to a patient suffering from myocardial infarction for one, two, three, four, or five days after the onset of the myocardial infarction. In a preferred embodiment, the compound as disclosed herein is administered to the patient for one, two, or three days after the onset of the myocardial infarction. In a preferred embodiment, the compound as disclosed herein is administered to the patient for three days following onset of the myocardial infarction. In another preferred embodiment, the compound as disclosed herein is administered to the patient for two days following onset of the myocardial infarction. In yet another preferred embodiment, the compound as disclosed herein is administered to the patient for one day following onset of the myocardial infarction.

In one embodiment of the present disclosure, the compound as disclosed herein is administered to a patient suffering from sepsis.

In one embodiment of the present disclosure, the compound as disclosed herein is administered to a patient suffering from sepsis for one, two, three, four, or five days after the onset of the sepsis. In a preferred embodiment, the compound as disclosed herein is administered to a patient suffering from sepsis for three, four, or five days after the onset of the sepsis. In another embodiment of the disclosure, the compound as disclosed herein is administered to a patient suffering from sepsis for as long as the level of S100A8/A9 in the blood of said patient is elevated, as compared to the normal concentration range of S100A8/A9 in the blood of healthy subjects.

In one embodiment of the present disclosure, the compound as disclosed herein is administered to a patient suffering from myocarditis for one week, two weeks, three weeks, or four weeks after the onset of the myocarditis. In a specific embodiment of the disclosure, the compound is administered to a patient suffering from myocarditis for one week. In another specific embodiment of the disclosure, the compound is administered to a patient suffering from myocarditis for two weeks. In yet another specific embodiment of the disclosure, the compound is administered to a patient suffering from myocarditis for three weeks. In a final specific embodiment of the disclosure, the compound is administered to a patient suffering from myocarditis for four weeks.

In one embodiment of the disclosure, the level of S100A8/A9 in the blood of a patient is measured and compared to the level of S100A8/A9 in a healthy subject, wherein a level of S100A8/A9 in the blood of the patient higher than that of a healthy subject is indicative that administration of a compound of the disclosure is beneficial, and a level of S100A8/A9 in the blood of the patient similar to that of a healthy subject is indicative that a compound of the invention need not be administered.

In one embodiment of the present disclosure, the compound as disclosed herein is administered to a patient suffering from septic cardiomyopathy.

In one embodiment of the present disclosure, the compound as disclosed herein is administered to a patient suffering from sepsis-induced myocardial inflammation.

In one embodiment of the present disclosure, the compound as disclosed herein is administered to a patient suffering from myocarditis.

In one embodiment of the present disclosure, the compound as disclosed herein is administered to a patient suffering from viral myocarditis.

In one embodiment of the present disclosure, the compound as disclosed herein is administered to a patient suffering from autoimmune myocarditis.

In one embodiment of the present disclosure, a compound disclosed herein is administered to a patient in need thereof via an oral route, intravenous route, subcutaneous route, or an intramuscular route.

In one embodiment, the compound as disclosed herein is administered via oral administration.

In one embodiment, the compound as disclosed herein is administered via intravenous (i.v.) administration.

In one embodiment, the compound as disclosed herein is administered via subcutaneous administration.

In one embodiment, the compound as disclosed herein is administered via intramuscular administration.

### EXAMPLES

### Example 1: High S100A8/A9 Levels in Acute Coronary Syndrome (ACS) Patients are Associated with Cardiac Remodeling, Left Ventricular Dysfunction and Heart Failure

### Materials and Methods

### Study Population and Outcome

S100A8/A9 was measured in plasma collected within 24 h after an acute coronary event from a population of 524 patients consecutively admitted for suspected ACS to the Coronary Care Unit of Skane University Hospital Malmö between October 2008 and December 2012. ACS was defined as unstable angina or MI, diagnosed according to the universal definition of MI. Out of the initial 605 patients recruited into the study, 50 did not fulfil the diagnostic criteria for ACS and 31 were further excluded due to missing samples. Baseline information on smoking, diabetes, hypertension, and previous history of heart failure and ACS have been collected from the national Swedish Web-based system for Enhancement and Development of Evidence-based care in Heart disease Evaluated According to Recommended Therapies (SWEDEHEART). All patients provided a written informed consent. Incident hospitalization with a main HF diagnosis was recorded prospectively during follow-up by using data from the Swedish Hospital Discharge Register. Heart failure was identified by ICD-10 code 150. The study has been approved by the local ethics committee for human research in Lund, Sweden and was conducted according to the ethical guidelines of the Declaration of Helsinki. Informed consent was obtained from all patients upon inclusion into the study.

### Statistics

The Mann Whitney test for continuous variables and the chi-square test for dichotomous variables were used to compare patient characteristics between the different outcome groups. The associations between S100A8/A9 and incident HF were assessed in multivariate Cox proportional hazards analyses with 2 different adjustment models: Model 1 included age and gender; Model 2 included age, gender, and the CV risk factors that differed significantly between the different outcome groups at baseline. These factors included hypertension, diabetes, eGFR, previous HF, and previous ACS (Table 1). Skewed variables were logarithmically transformed before analysis. The correlations between biomarkers and echocardiographic parameters were analysed with the Spearman rank test. P values <0.05 were considered to be significant. The statistical analysis was performed using SPSS 23.0 (IBM software, Armonk N.Y.) or Prism (version 7, GraphPad Software Inc., CA, USA).

### Patient Echocardiography

A subgroup of 113 patients older than 75 years of age completed a follow-up echocardiography 1 year after inclusion. All examinations have been analysed using the Xcelera software (Philips) by a single examiner blinded to the clinical data. Left ventricle ejection fraction (LVEF) was measured according to Simpson's biplane method in the apical 4-chamber and 2-chamber views. Due to poor image quality and missing frames, measurements of LVEF at 1-year post-ACS were performed in 99 patients, and measurements of the left ventricle end-systolic volume (LVESV) and left ventricle end-diastolic volume (LVEDV) at 1 year in 108 patients.

### Biochemical Analysis

Plasma samples were collected in EDTA-coated tubes within 24 hours after admission. The blood was centrifuged at 3000 g for 10 minutes, and plasma was aliquoted and stored at −80° C. before analysis. Troponin T (TnT) and cystatin C in plasma were measured by the certified clinical laboratory of Skane University Hospital Malmö. The Chronic Kidney Disease Epidemiology Collaboration (CKD-EPI) formula was used to calculate eGFR based on cystatin C, age and sex. S100A8/A9 analysis was performed by using an ELISA kit, according to manufacturer's instructions (BMA Biomedicals, Augst, Switzerland).

### Results

As elevated S100A8/A9 post-MI has previously been related to an increased risk for recurrent MI and cardiovascular death, we investigated whether the S100A8/A9 is also associated with the development of heart failure (HF) in ACS patients. We measured S100A8/A9 in plasma collected within 24 hours after the acute event from 524 patients presenting with an ACS. During follow-up, 41 of these patients developed clinical HF, defined as hospitalization with a main HF diagnosis after discharge. The patients who suffered an HF event were older and had a higher prevalence of hypertension, diabetes, renal dysfunction, previous ACS or a previous HF diagnosis (Table 1). In a Kaplan Meyer analysis with log-rank test, patients with S100A8/A9 in the highest tertile had a significantly higher risk for incident HF compared to the first two tertiles (FIG. 6; P for trend=0.006). These results were further confirmed in a Cox proportional regression analysis adjusted for age and sex, showing a 1.34 (95% CI 1.05-1.69) higher risk for HF hospitalization for each SD increase in baseline S100A8/A9 values (Table 2, Model 1). The relationship narrowly lost significance after further adjustment for potential clinical confounders that differed between the two groups at baseline (Table 2, Model 2). Follow-up echocardiographic examinations were available in 113 of these patients. In these patients, higher S100A8/A9 at baseline was correlated with lower left-ventricular ejection fraction (EF) and larger end-systolic volume (ESV) at 1 year after the ACS (Table 2).

TABLE 1
Differences in clinical characteristics at baseline between ACS patients with and without
incident HF during follow-up.

| | | Patients with | Patients without | |
|---|---|---|---|---|
| | All patients | incident HF | incident HF | |
| Characteristics | N = 524 | N = 41 | N = 483 | P |
| Age (years) | 67 (59-77) | 77 (71.5-84.5) | 66 (58.0-75.8) | <0.001 |
| Male gender, n (%) | 384 (73.1%) | 28 (68.3%) | 356 (73.6%) | n.s. |
| Hypertension, n (%) | 285 (54.3%) | 34 (82.9%) | 251 (51.9%) | <0.001 |
| Smoking, n (%) | 132 (25.1%) | 10 (24.4%) | 122 (25.2%) | n.s. |
| Diabetes, n (%) | 126 (24.0%) | 20 (48.8%) | 106 (21.9%) | <0.001 |
| BMI (kg/m²) | 26.9 (24.3-29.8) | 27.2 (23.9-29.8) | 26.9 (24.3-29.8) | n.s. |
| eGFR (mL/min/1.73 m²) | 72.0 (53.1-94.6) | 46.0 (31.6-64.2) | 73.9 (55.8-96.3) | <0.001 |
| Index cardiac event | | | | |
| STEMI | 180 (34.4%) | 11 (26.8%) | 169 (34.9%) | n.s. |
| NSTEMI | 295 (56.3%) | 24 (58.5%) | 271 (56.0%) | n.s. |
| UA | 50 (9%) | 6 (14.63%) | 44 (9.1%) | n.s. |
| Previous cardiac event | | | | |
| HF, n (%) | 54 (10.3%) | 15 (36.6%) | 39 (8.0 %) | <0.001 |
| ACS, n (%) | 152 (29.0%) | 19 (46.3%) | 133 (27.5%) | <0.001 |
| TnT (ng/L) | 364 (62-1290) | 496 (94-1248) | 356 (54-1300) | n.s. |
| S100A8/A9 (μg/mL) | 4.9 (3.4-7.9) | 6.9 (4.2-11.9) | 4.9 (3.3-7.5) | 0.002 |

STEMI, ST elevation myocardial infarction; NSTEMI, non-ST elevation myocardial infarction; UA, unstable angina; HF, heart failure; ACS, acute coronary syndrome; BMI, body mass index; TnT, Troponin T; eGFR, estimated glomerular filtration rate.

TABLE 2
Correlation between plasma S100A8/A9 during the acute post-ischemic
phase, hospitalization for heart failure, and cardiac function at 1 year after an acute
coronary syndrome.

| | | | | | LVEF | | ESV | | EDV | |
|---|---|---|---|---|---|---|---|---|---|---|
| | | Heart Failure | | | 1 year post- | | 1 year post- | | 1 year post- | |
| | | (N = 41)* | | | ACS | | ACS | | ACS | |
| Biomarker | Models | HR^{‡} | CI for HR | P | r^{†} | P | r^{†} | P | r^{†} | P |
| S100A8/A9 | 1 | 1.34 | 1.05-1.69 | 0.019 | −0.3322 | 0.001 | 0.201 | 0.037 | 0.11 | n.s. |

| | | | |
|---|---|---|---|
| 2 | 1.30 | 0.98-1.72 | 0.067 |

*Incident hospitalization with a clinical diagnosis of heart failure during follow-up
^{‡}Multivariate Cox proportional hazards analyses of the relationship between S100A8/A9 at inclusion and incident heart failure. The hazard ratio (HR) is expressed per one standard deviation increase in S100A8/A9 values.
^{†}Spearman correlation coefficient
Adjustment models:
Model 1: age and gender;
Model 2: age, gender and CV risk factors that differed at baseline between the patients with and without incident HF during follow-up (hypertension, diabetes mellitus, eGFR, previous HF, previous ACS)
ACS, acute coronary syndrome; Cl, confidence interval; EDV, end-diastolic volume;
eGFR, estimated glomerular filtration rate; ESV, end-systolic volume; HR, hazard ratio;
hsCRP, high sensitivity C-reactive protein; LVEF, left ventricle ejection fraction.

### Conclusion

Our data reveals that high S100A8/A9 levels in acute coronary syndrome (ACS) patients are associated with cardiac remodeling, left ventricular dysfunction and heart failure during follow-up. Patients with S100A8/A9 values in the highest tertile (>6.7 μg/mL in our cohort) had a much higher incidence of heart failure, while the risk for heart failure in the first two tertiles was similarly low.

### Example 2: Short-Term S100A9 Blockade Significantly Improves Cardiac Function after Permanent Myocardial Ischemia

### Materials and Methods

### Synthesis of the Compounds of the Present Disclosure

The syntheses of the compounds of the present disclosure have been published in WO 2014/184234.

### Inhibition of S100A9 Binding to RAGE and TLR4 by ABR-238901

The S100A9 inhibitor ABR-238901 was a gift from Active Biotech, Lund, Sweden. ABR-238901 is a heteroaryl-sulfonamide derivative identified in a high-throughput screening program to potently inhibit soluble S100A9 binding to RAGE and TLR4. The screening was performed in several steps and yielded S100A9 inhibitors that were non-toxic, metabolically stable and orally available. The structure of the molecule is presented in FIG. 7. Inhibition of S100A9 binding to RAGE and TLR4/MD2 by ABR-238901 was tested by surface plasmon resonance in a Biacore 3000 system (GE Healthcare, Uppsala, Sweden). Briefly, 50 nM of recombinant human S100A9 was injected over immobilized RAGE and TLR4 (2 min at 30 μL/min) in the presence of increasing concentrations of ABR-238901, ranging from 0.0488 to 100 μM. The magnitude of the responses measured at the late dissociation phase (t 350 s) in the presence of ABR-238901 (B_{ABR-238901}) were expressed as percentage of response in the absence of competitor (Bo), and plotted against the concentration of ABR-238901 (FIG. 7b-c). The curves were fit to a sigmoidal dose-response model with variable slope in Prism GraphPad for calculation of IC₅₀. The analysis confirmed that ABR-238901 inhibits the binding of S100A9 to both RAGE and TLR4, with IC₅₀ values of 2.7 μM and 2.1, respectively.

### Animal Models and Treatment

All mouse studies were approved by the Animal Research Ethics Committee of Lund University. C57BL/6NRJ mice were purchased from Janvier Labs, C57BL/6-Tg(Nr4a1-EGFP/cre)820Khog/J (Nur77-EGFP) and B6;D2-Tg(Myh6*-mCherry)2Mik/J (α-MHC-mCherry) were purchased from the Jackson Laboratory. Nur77-EGFP mice express the fluorescent EGFP protein under the Nur77 promoter. α-MHC-mCherry mice express the red fluorescent Cherry protein under the mouse alpha myosin heavy-chain promoter in cardiomyocytes. We used 8-12 weeks old female mice in all experiments. MI was induced by left coronary artery ligation using a minimal thoracotomy model. Mice were initially anesthetized with 4% isoflurane and the surgical procedure was performed under 1.7% isoflurane. Following heart exposure outside of the thorax through a small thoracic incision, the left coronary artery was permanently ligated using silk 6.0 suture, the heart repositioned, and the thorax was closed with 6.0 prolene monofilament suture.

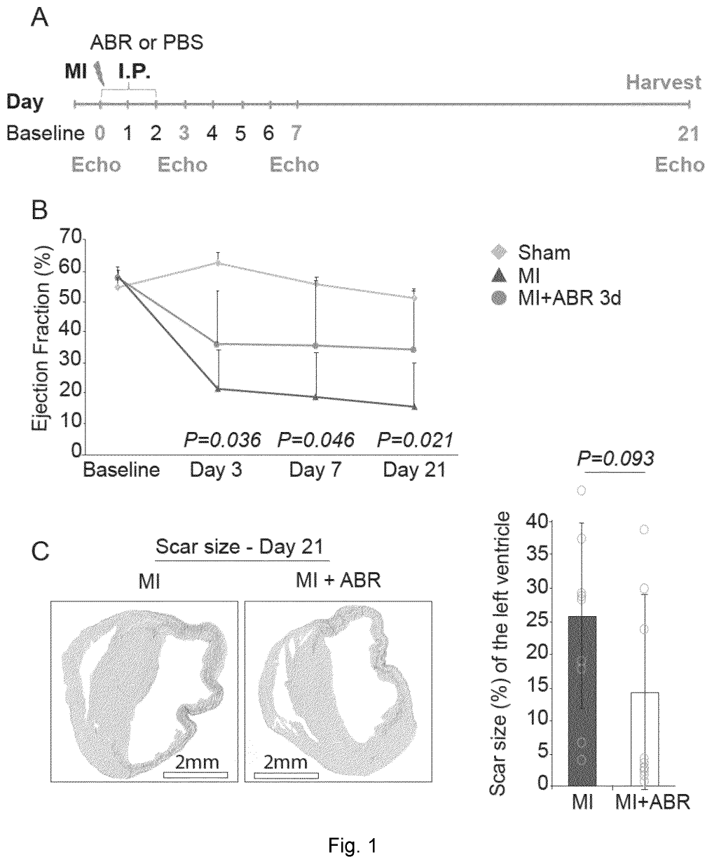

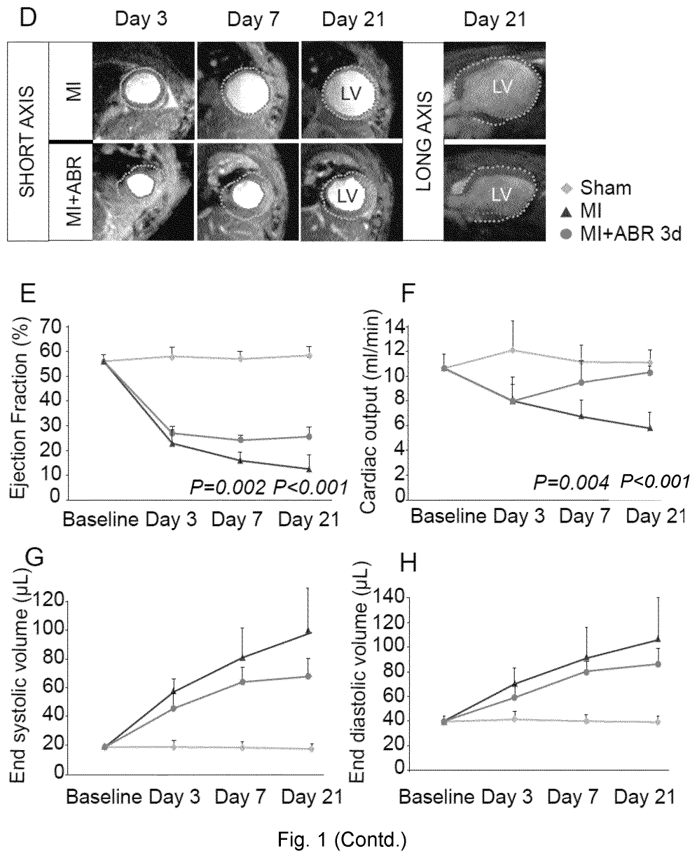

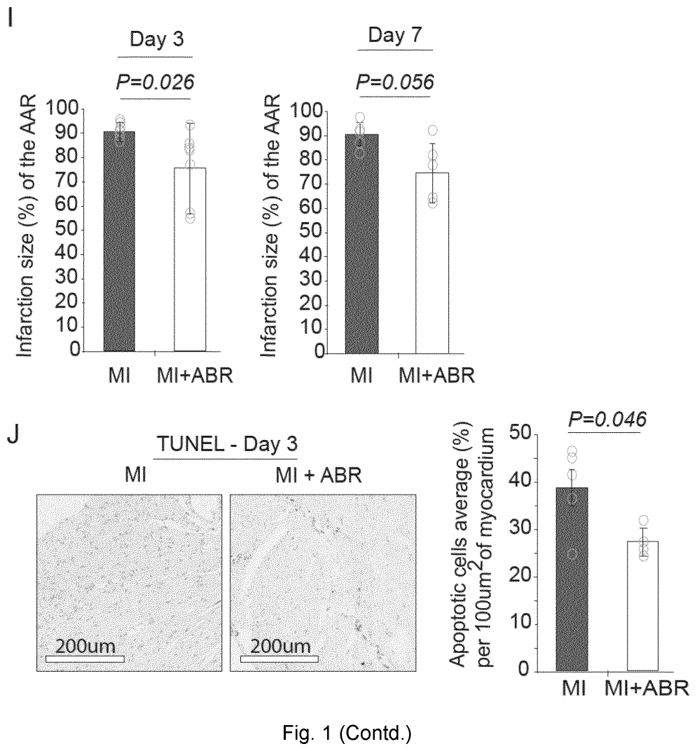

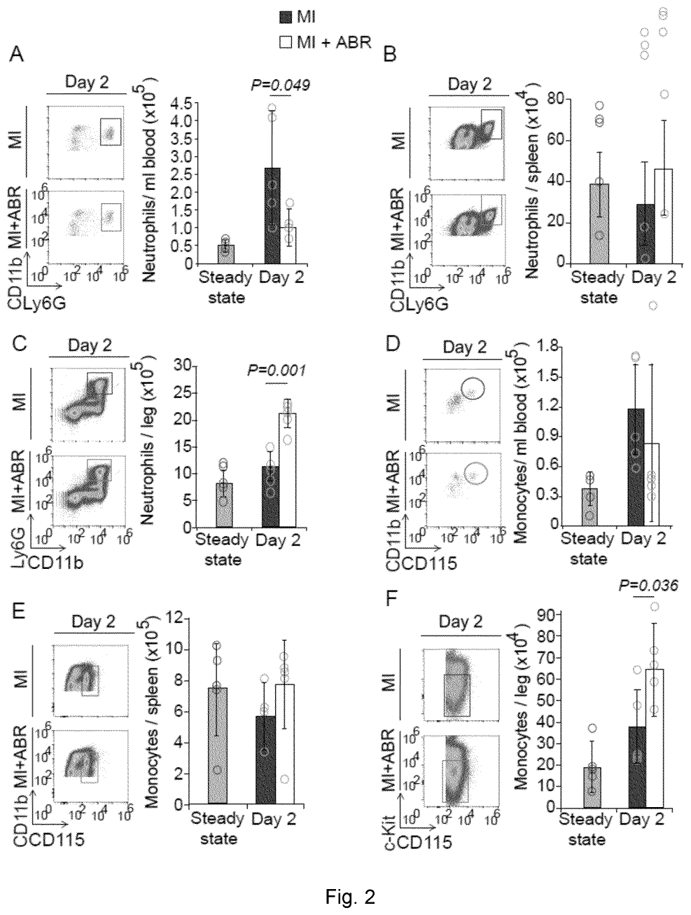

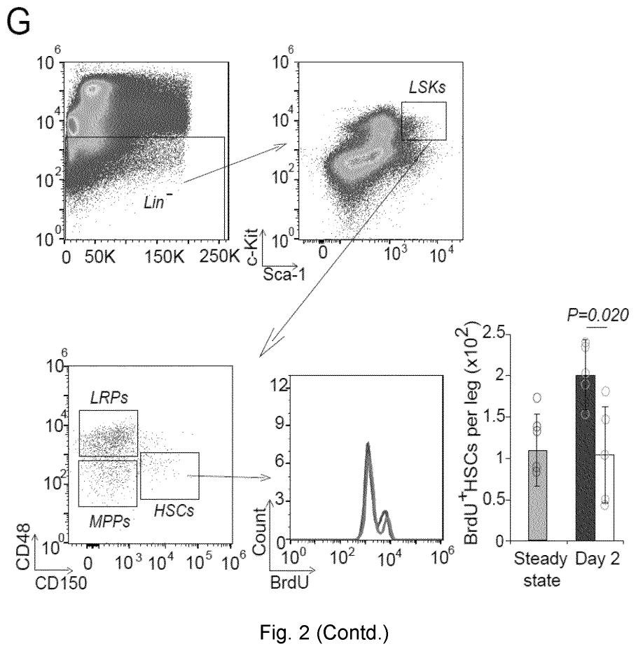

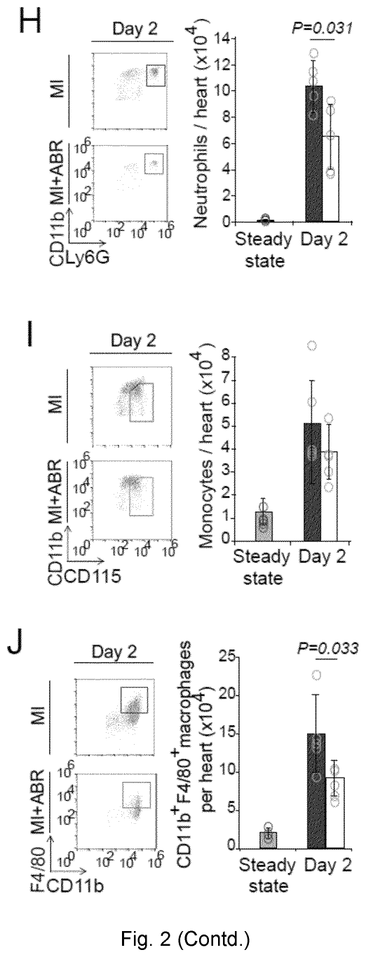

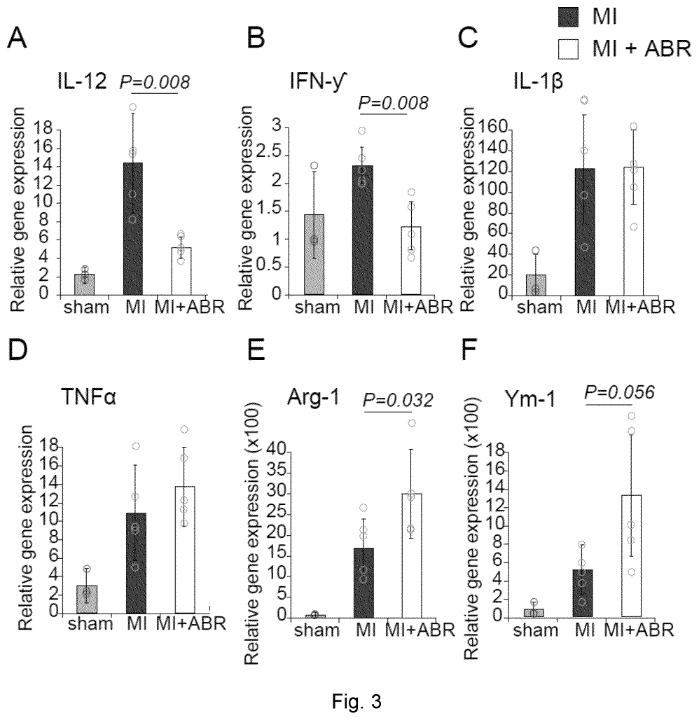

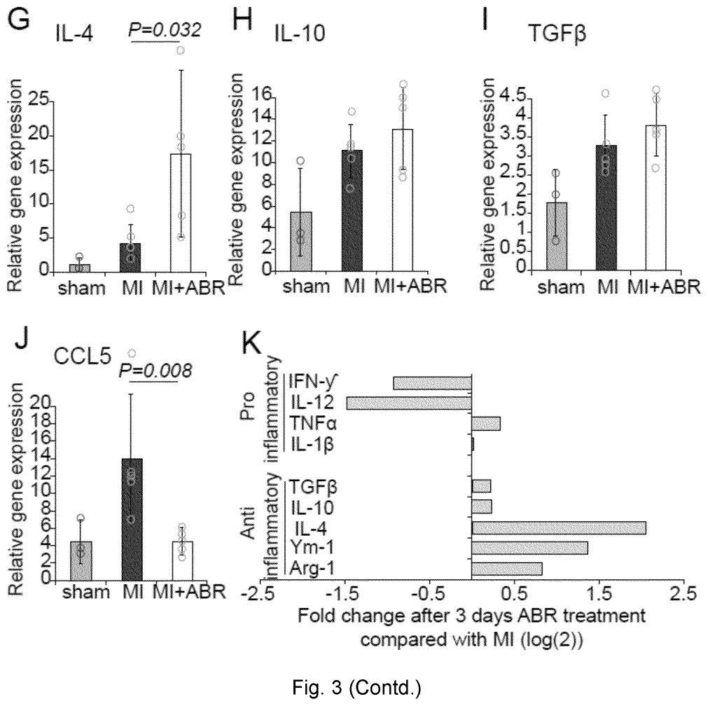

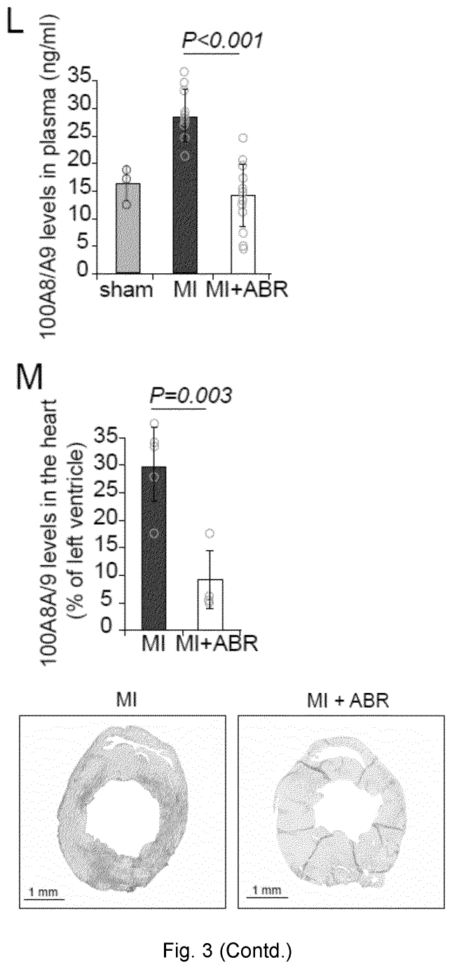

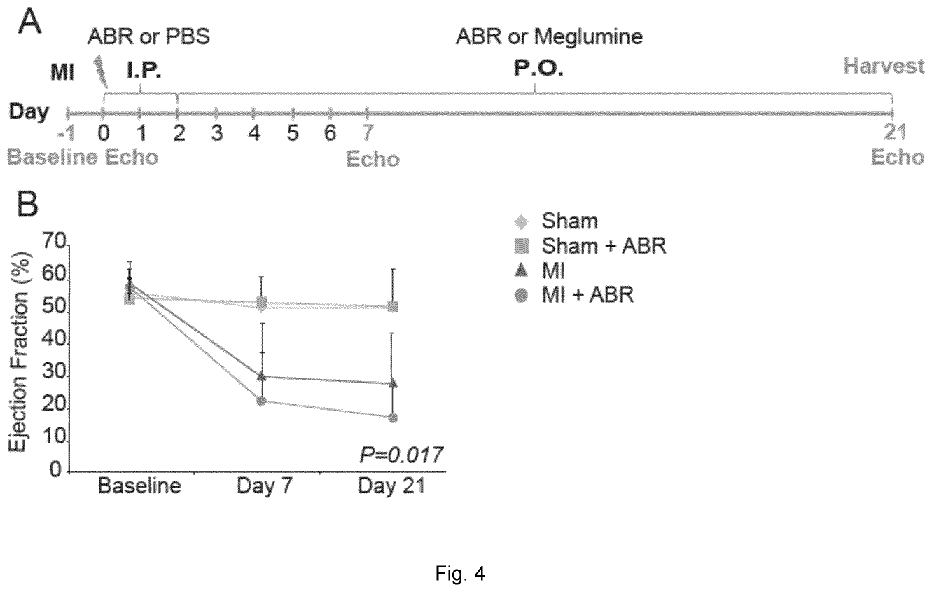

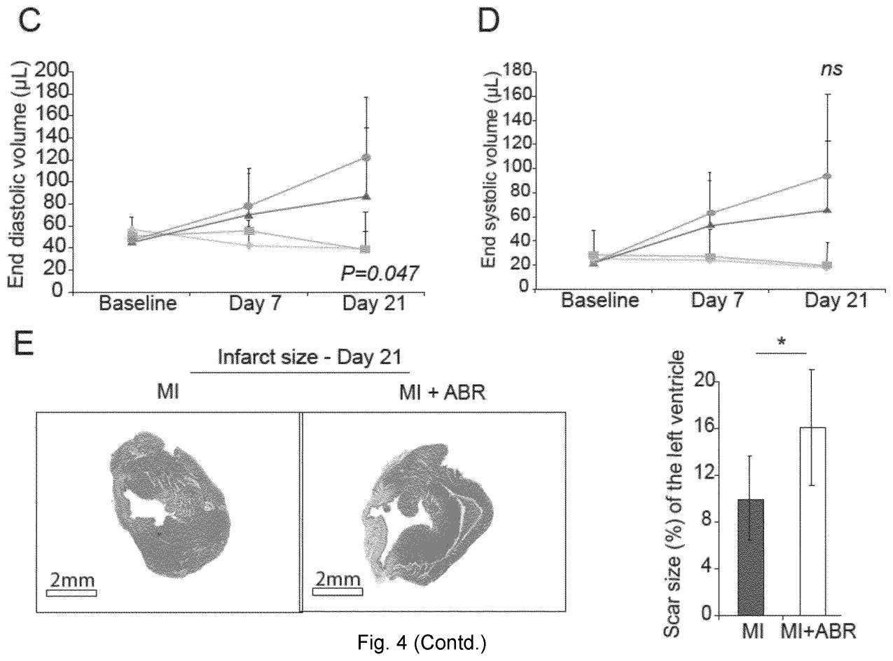

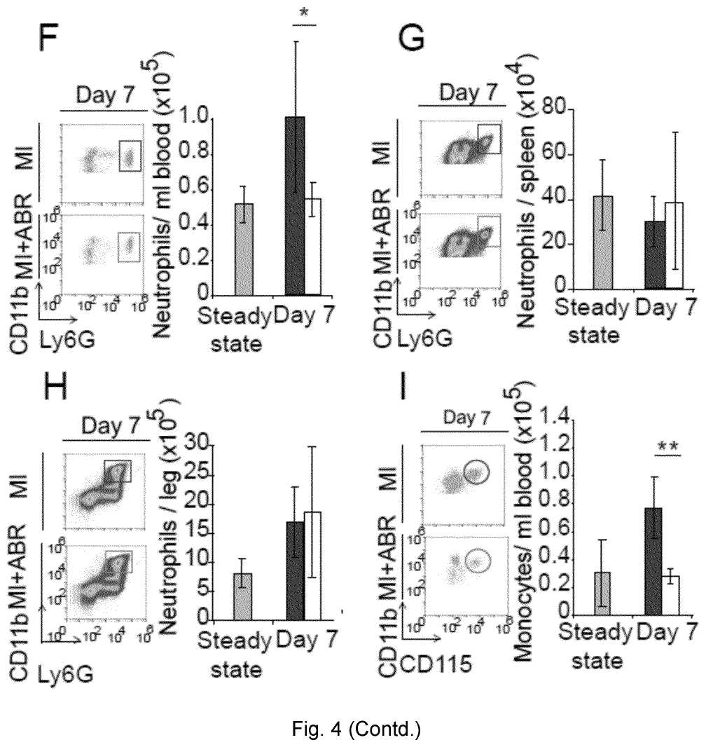

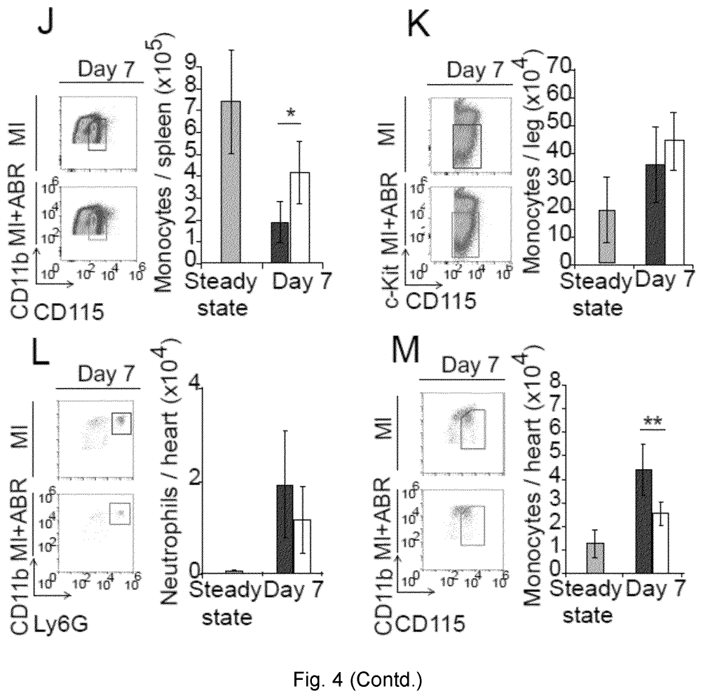

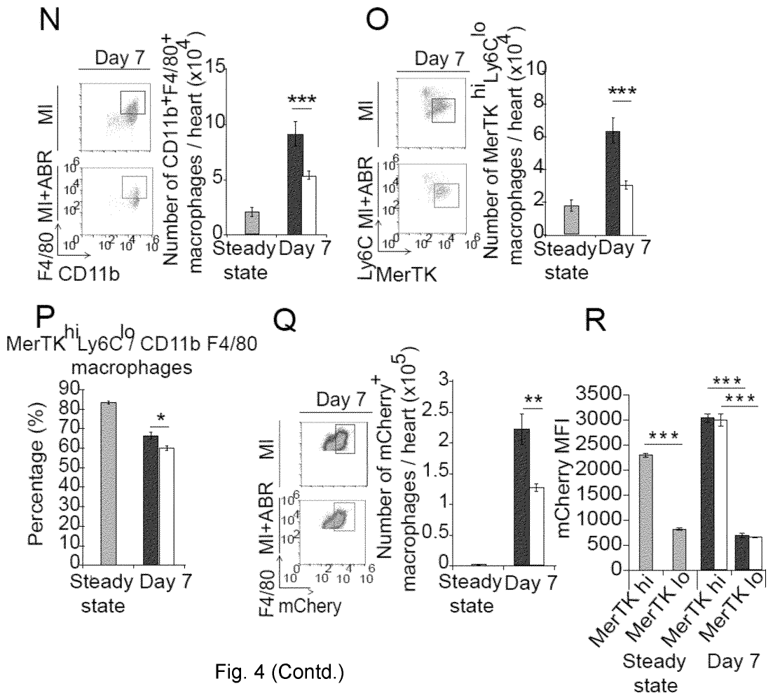

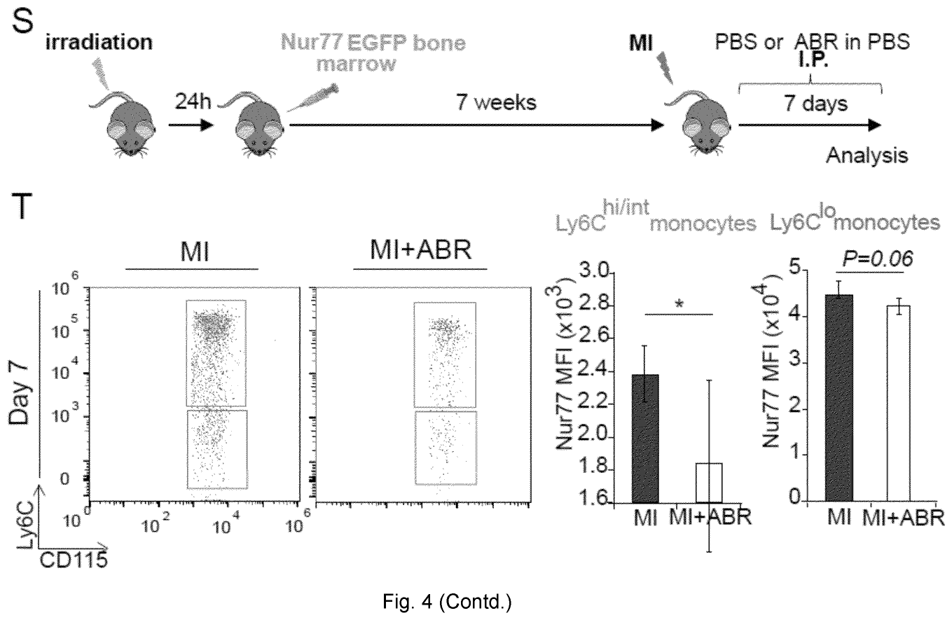

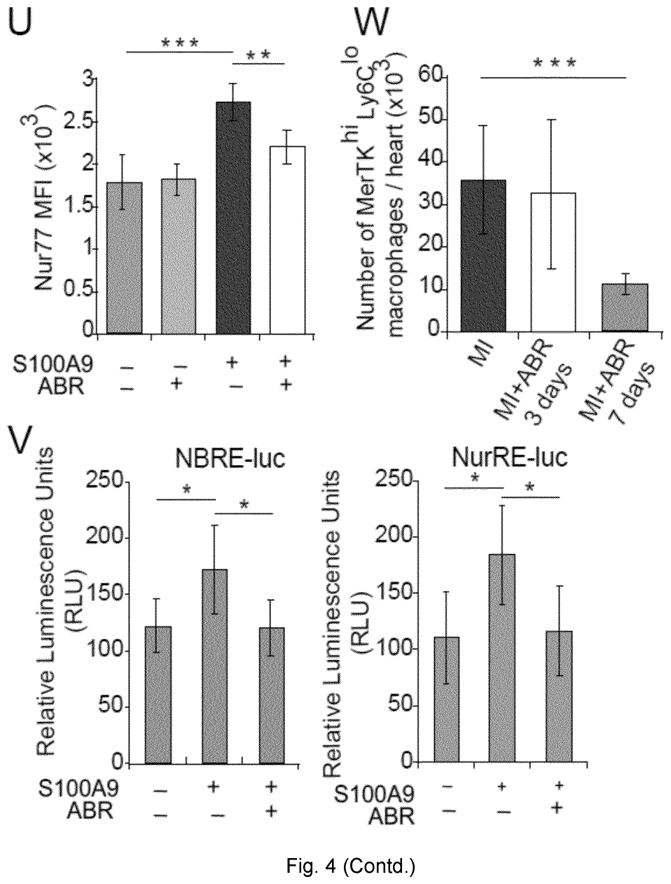

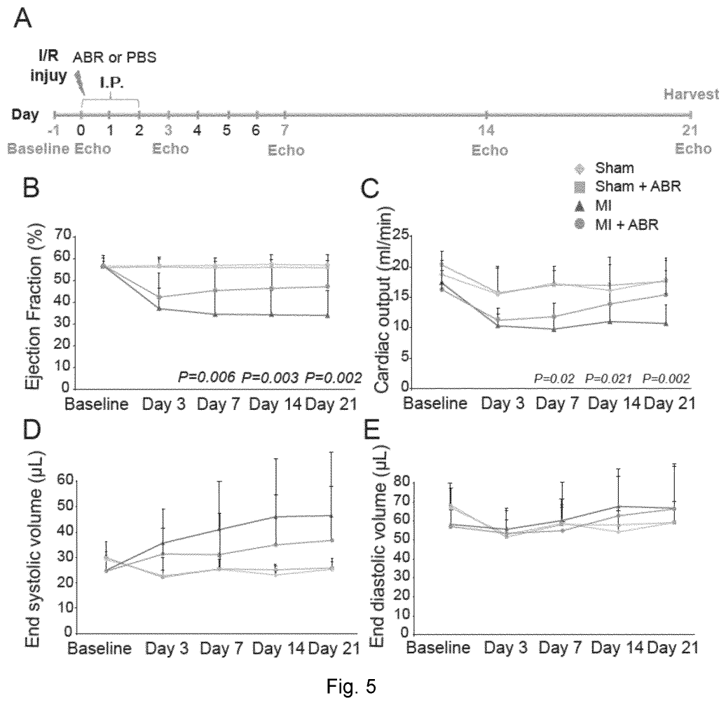

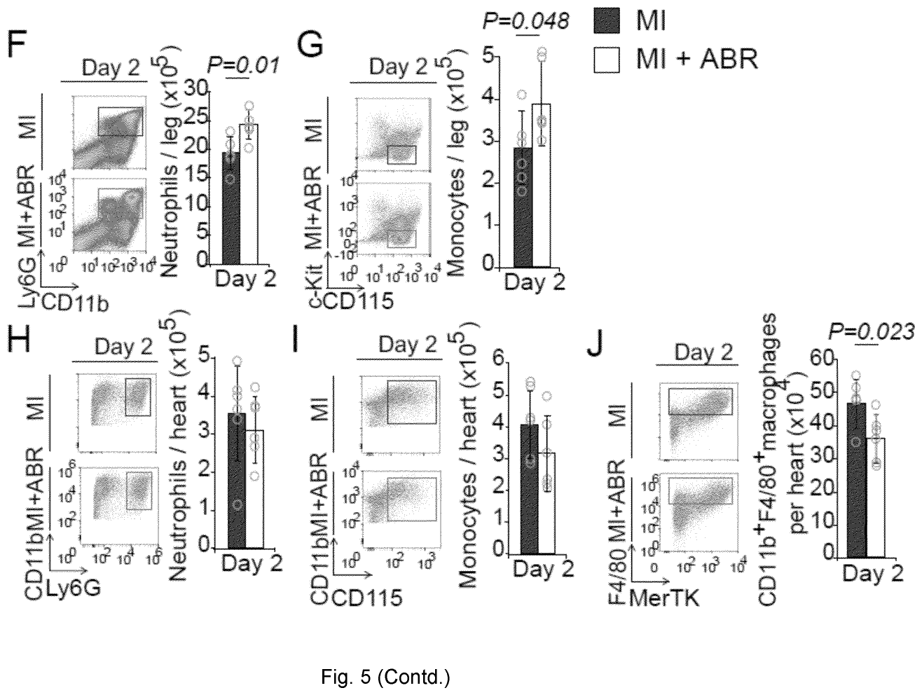

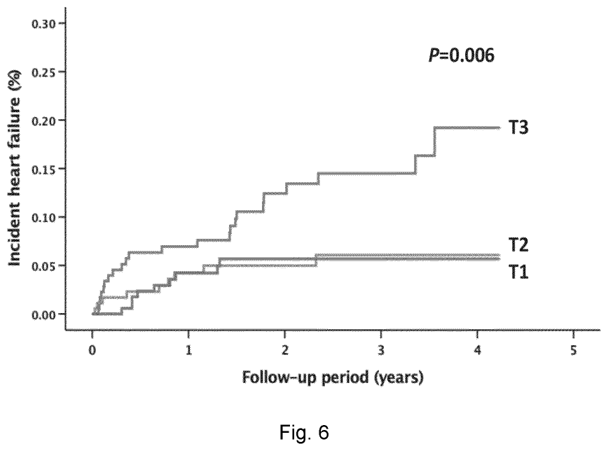

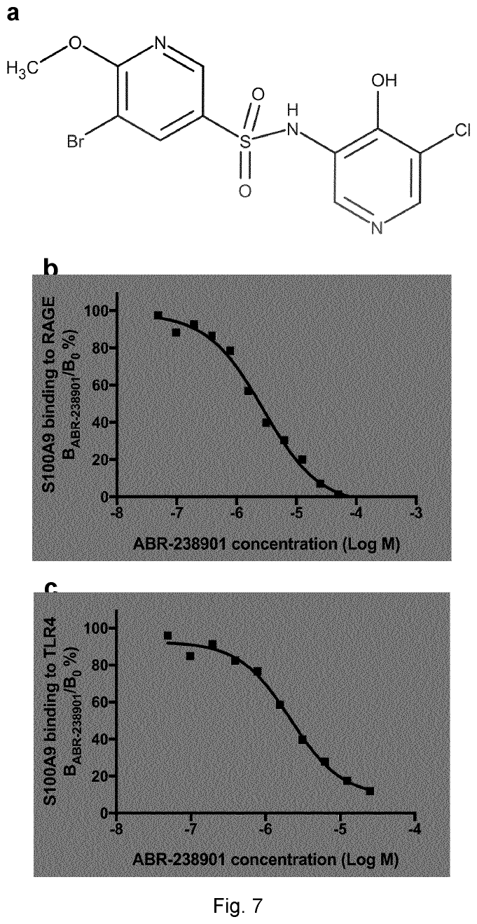

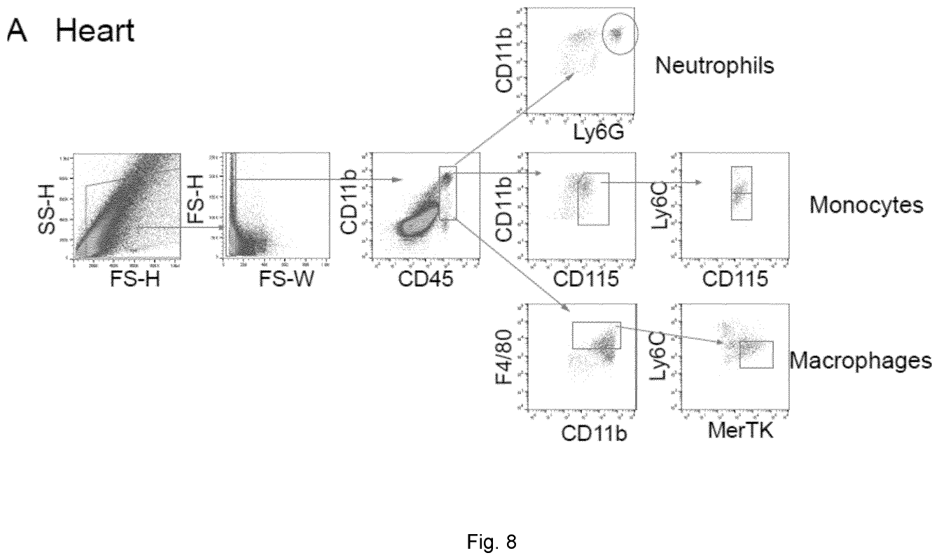

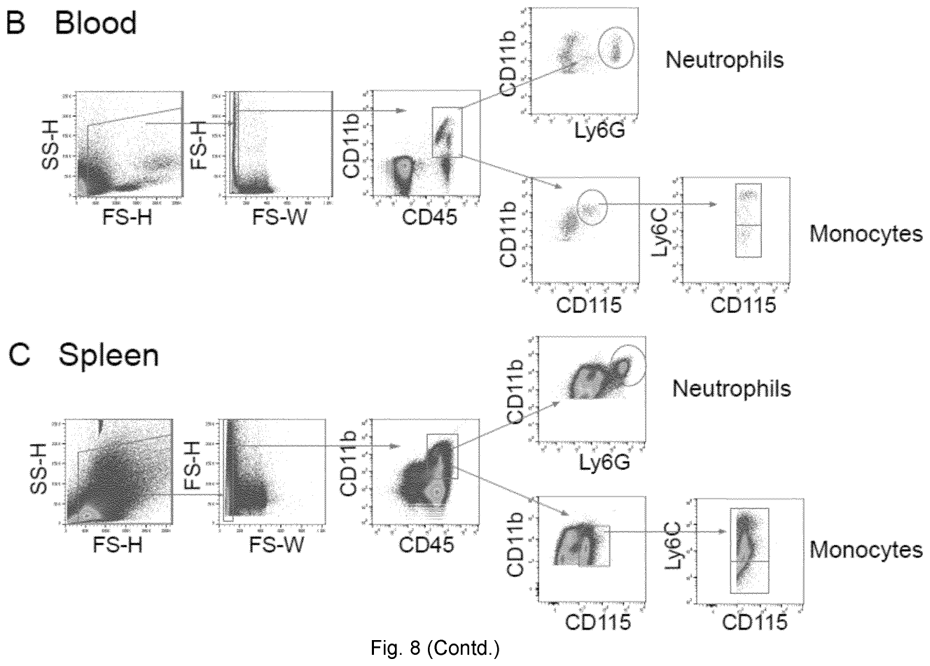

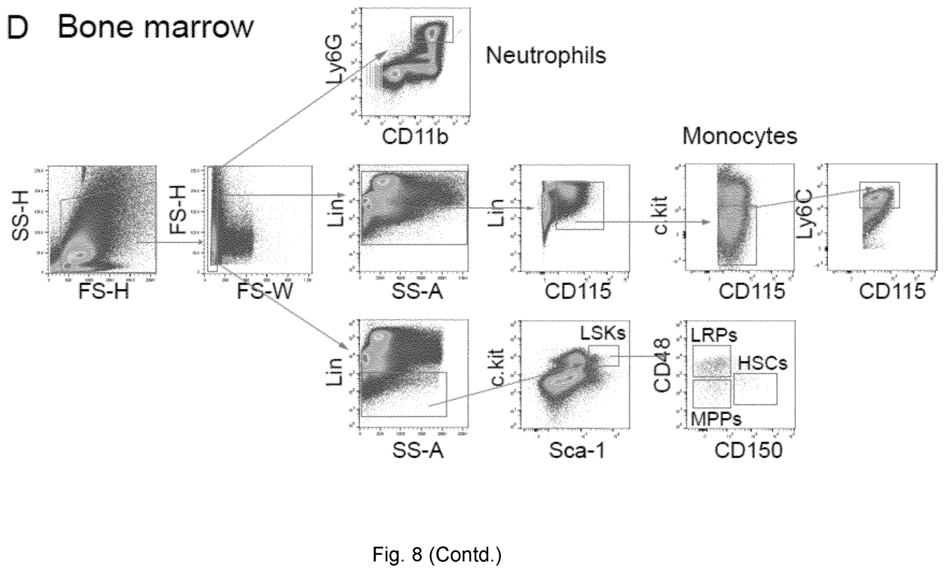

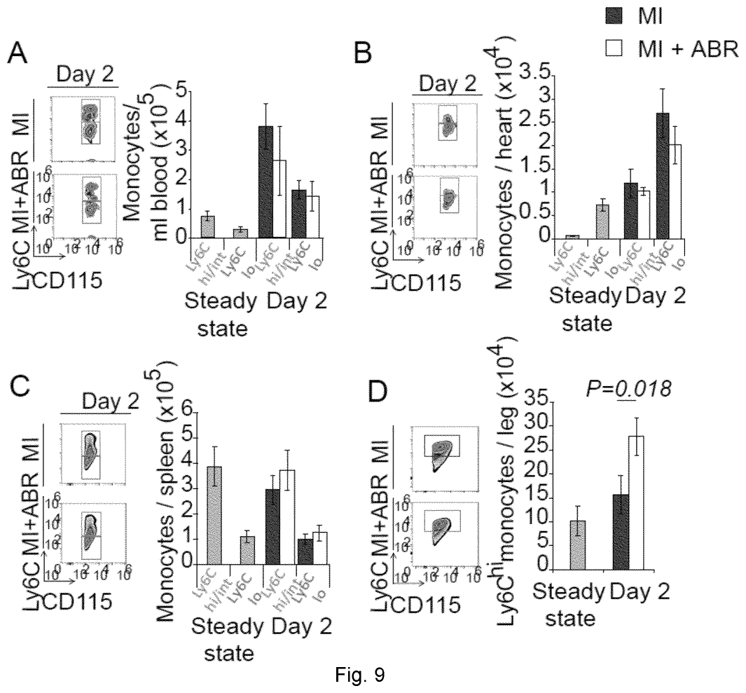

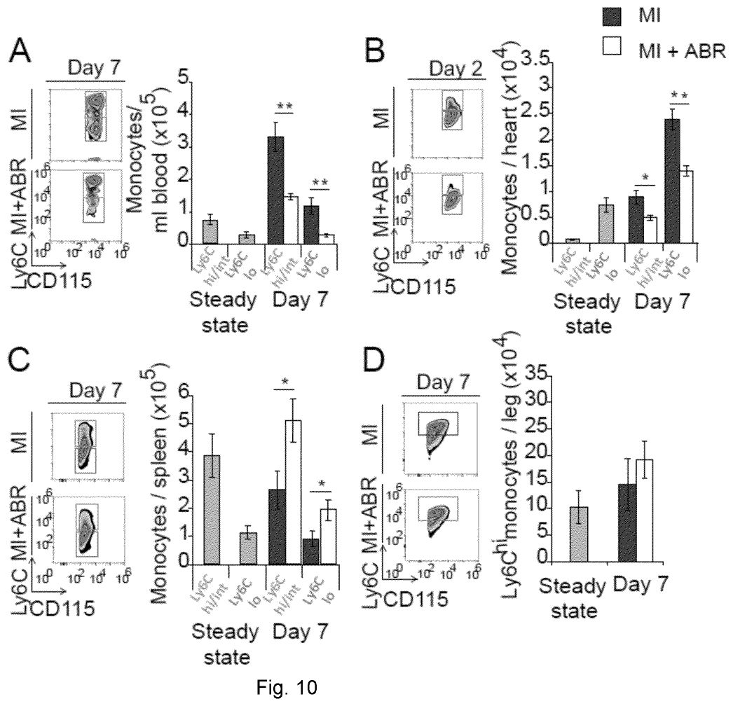

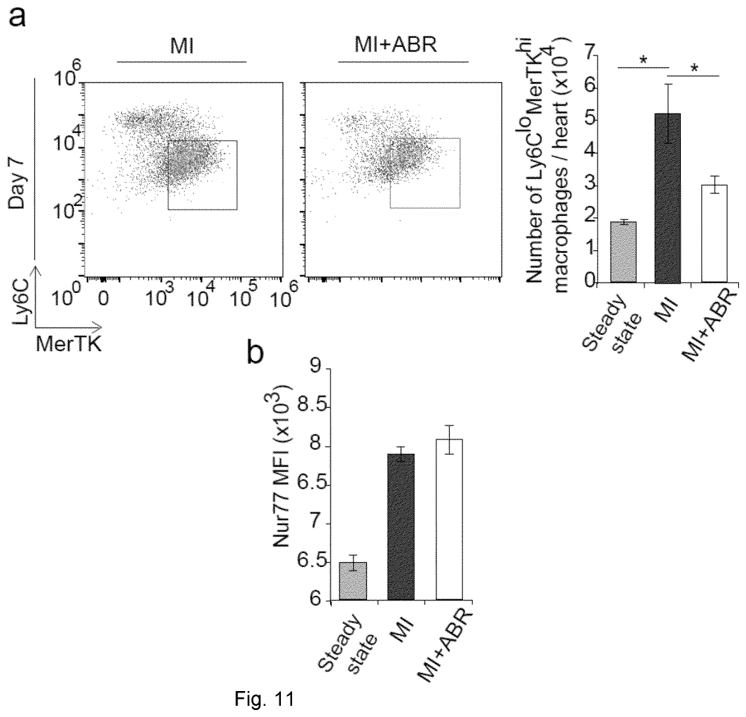

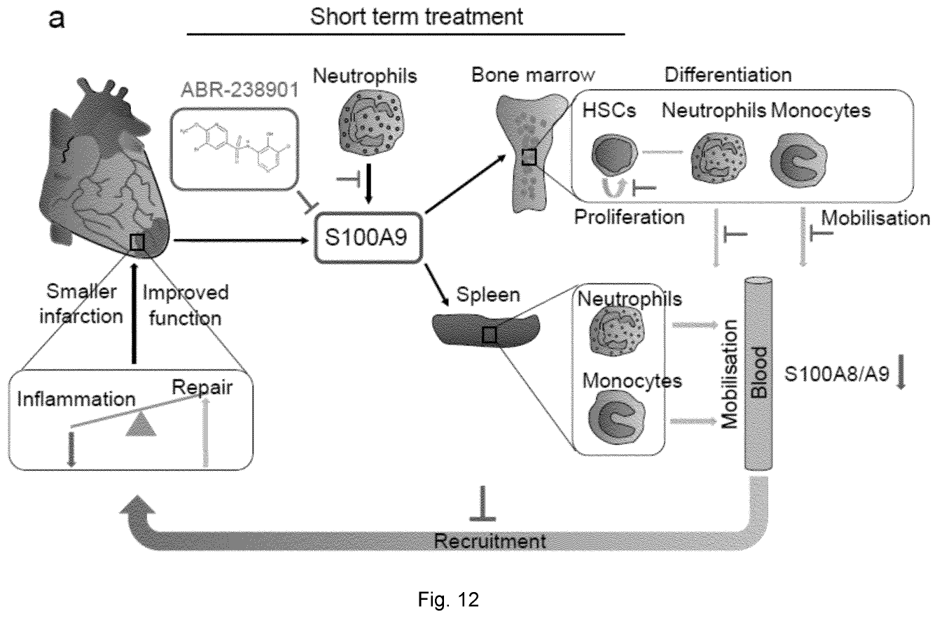

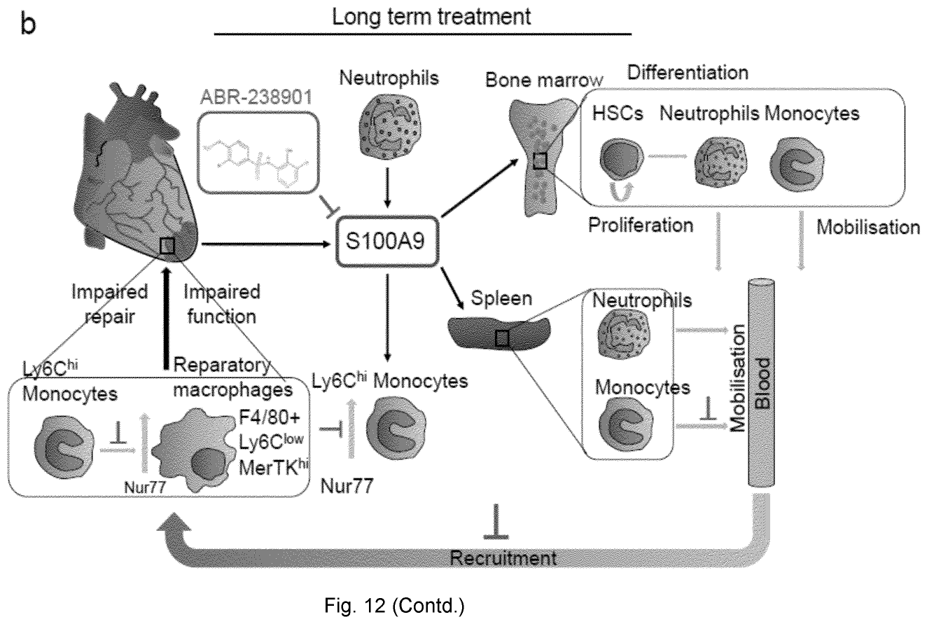

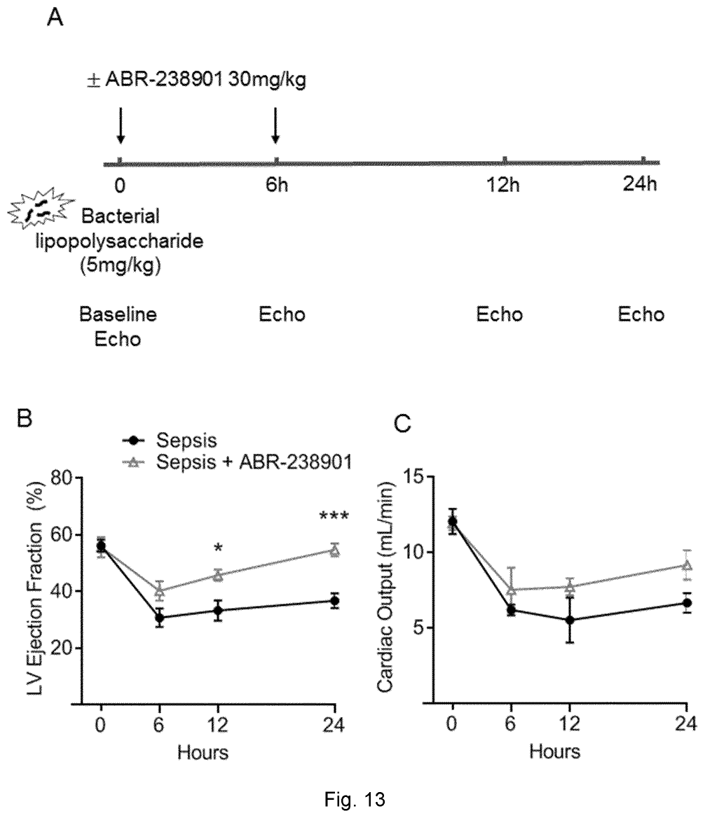

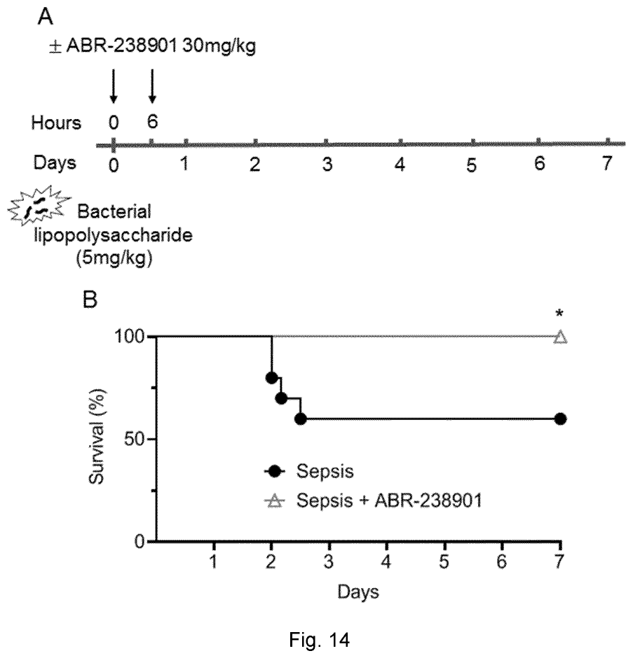

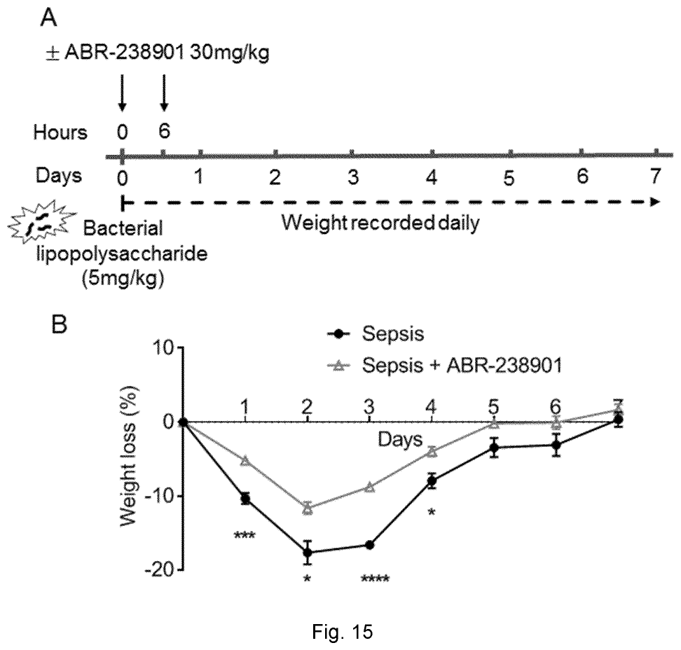

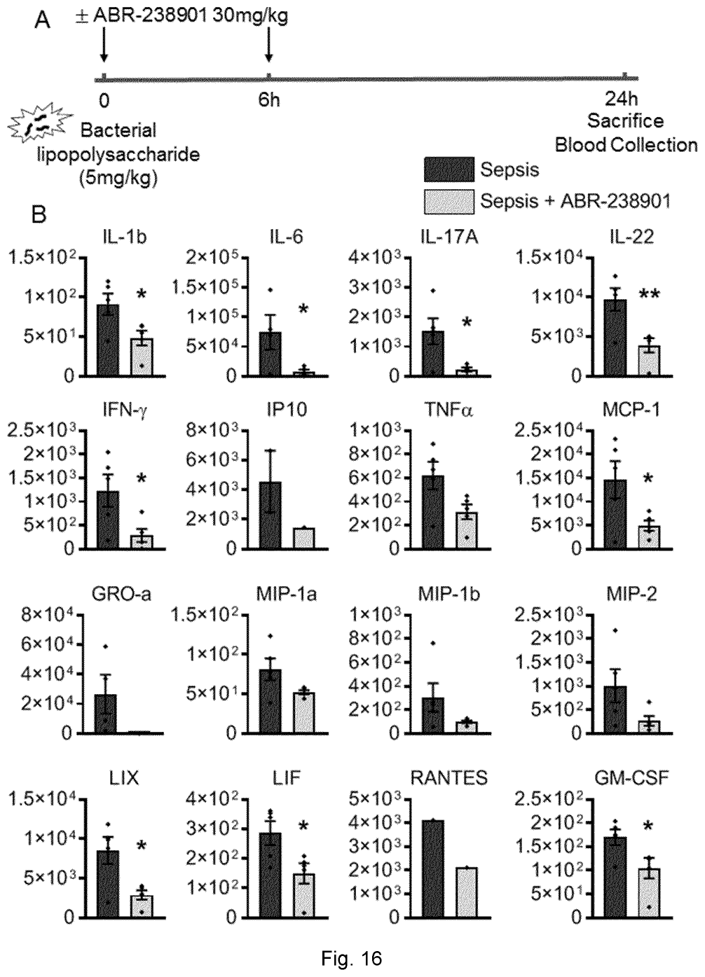

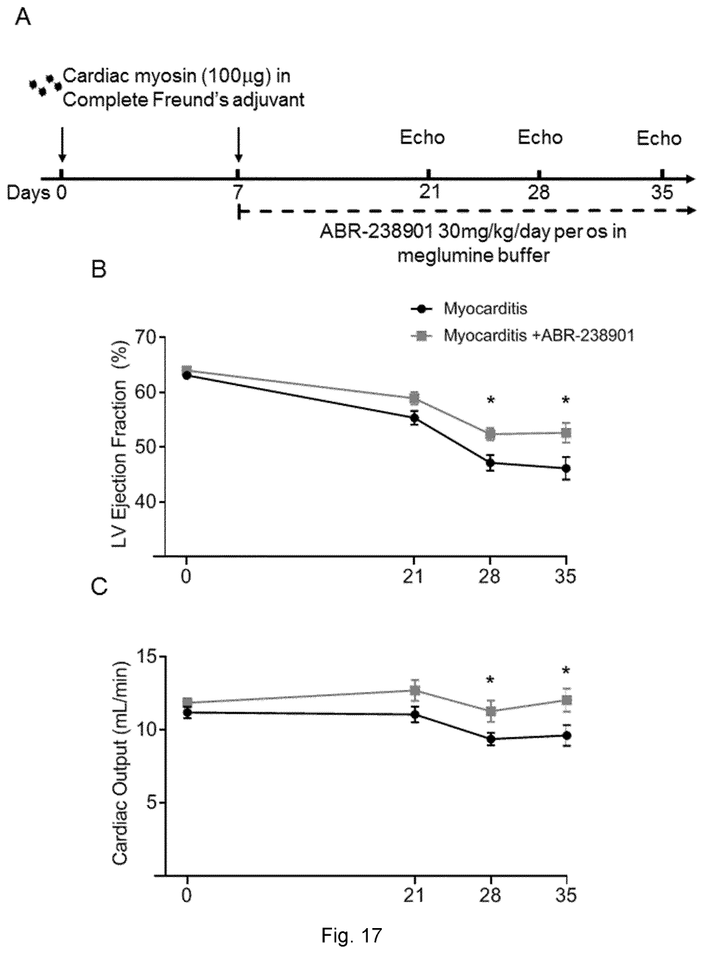

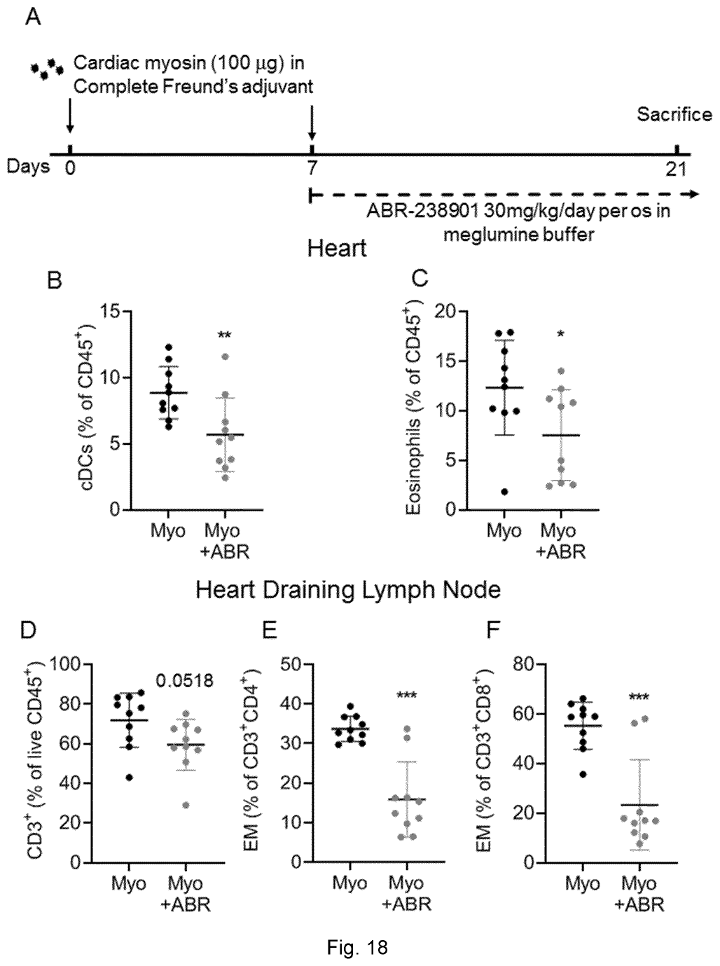

For short-term experiments, a daily dose of 30 mg/Kg ABR-238901 was diluted in PBS and administered by daily i.p. injections for the first 3 days post-MI, starting immediately after ischemia induction (days 0, 1 and 2) (FIG. 1A). Since the S100A9 receptors RAGE, TLR4 and EMMPRIN are located on the cell surface, ABR-238901 (and similar compounds described in the present invention) is thought to mainly inhibit the effects of extracellular S100A9.

### Bone Marrow Transplantation

Eight-week-old C57BL/6NRJ female mice were lethally irradiated with a total dose of 910 cGy. One day after irradiation, 1×10⁶ total bone marrow cells harvested from donor C57BL/6-Tg(Nr4a1-EGFP/cre)820Khog/J (Nur77-EGFP) mouse and re-suspended in PBS were administered to each recipient mouse via the tail vein. Recipient mice were treated with neomycin in drinking water (2 g/L) for two weeks. Blood samples were collected from all the recipient mice at four weeks after bone marrow transfer, to confirm the success of bone marrow reconstitution by flow cytometry. The mice were used for experiments 6 weeks after bone marrow transfer (FIG. 4S).

### Mouse Echocardiography

Ultrasound measurements were performed at day −1 (baseline), 7, and 21 to assess cardiac function using a Vevo 770 echocardiographic imaging system with a 707B probe (VisualSonics, Toronto, Canada). In selected experiments, additional assessment was performed on days 3 and 14. The mice were anesthetized using 1-3% isoflurane and hair was carefully removed using depilatory cream. To maintain body temperature, the mice were placed in supine position on a heated surface (37° C.). Two-dimensional M-mode images were recorded in the parasternal short-axis view at the mid-papillary level. Five complete cardiac cycles were traced to calculate fractional shortening, ejection fraction, left ventricular systolic volume, and left ventricular diastolic volume by using the VisualSonics software.

### Histology

At 21 days after MI, the hearts were perfused with ice cold PBS and excised under ketamine/xylazine anesthesia. Subsequently the hearts were fixed in 4% (w/v) paraformaldehyde and embedded in paraffin. The hearts were sectioned transversely from the apex to the level of the suture, and 6 μm thick sections were collected at 390 μm intervals. The sections were mounted on glass slides and stained with Van Gieson or Masson trichrome stain for quantitative analysis of scar size. Alarmin S100A8/A9 was detected using mouse anti-S100A8/A9 IgG1 antibody (Abcam, ab 130234, 1:100 dilution, O/N 4° C.). Goat anti-mouse IgG1 HRP-conjugated secondary antibody (Abcam, ab 97240, 1 h RT) was used and the staining was developed with ImmPACT™ DAB (Vector, SK-4105) chromogen as substrate. The slides were subsequently scanned using an Aperio digital pathology slide scanner (Leica Biosystems), and images were captured with the ImageScope software (Leica Biosystems). Left ventricle area and the infarcted area were measured on each section by using the BioPix iQ 2.5.0 software (Biopix AB). The size of the post-MI myocardial scar was expressed as percentage of the left ventricle volume below the coronary ligature.

### Magnetic Resonance Imaging (MRI)

Magnetic resonance measurements were performed on a 9.4 T horizontal magnet (Agilent, Santa Clara, USA) equipped with Bruker BioSpec AVIII electronics operating with ParaVision 6.0.1 (Bruker, Ettlingen, Germany). The experiments were conducted under general anaesthesia using a mixture of isoflurane (2%) and 98% O₂ via a nose cone. Rectal temperature, ECG signal, and breathing rate were monitored throughout the measurements (SA instruments, Stony Brook, USA). During the experiment, the mouse was positioned prone in an animal holder and kept warm to maintain constant body temperature at 36-37° C. ECG and respiration triggered FLASH sequence was used to acquire cinematographic MR image series of the beating heart. To ensure full coverage of the cardiac cycle, 24 frames were acquired. Nine contiguous left ventricular (LV) short-axis slices (slice thickness 1 mm) were acquired, ensuring a full coverage of the ventricles from the base to the apex. Short-axis slices were complemented with 4- and 2-chamber long-axis views. The imaging parameters were field of view, matrix 192×192, echo time 2.1 ms, repetition time 6 ms and flip angle 15 degrees, 1 average. All MR image analysis was done using the freely available software Segment version 2.2 R6887.

### Mouse S100A8/A9 Heterodimer ELISA

Plasma levels of S100A8/A9 heterodimer was measured by DuoSet®ELISA (R&D Systems, DY8596-05), according to manufacturer instructions. All the samples were diluted 1:100 prior to analysis. Biotinylated goat anti-mouse S100A8/A9 antibody was used as capturing antibody and streptavidin-HRP was used for detection. Colour was developed using 1-Step™ Ultra-TMB-ELISA (Thermo Scientific, Cat. No. 34028).

### Cell Isolation and In-Vitro Murine Macrophage and Monocyte Culture

To obtain bone marrow-derived monocytes, bone marrow cells were isolated from femur and tibia of wild-type mice (C57BL/6NRJ) (n=3). Cells were cultured in RPMI-1640 (GIBCO Invitrogen) with 100 U/ml penicillin/streptomycin (GIBCO Invitrogen), 10% heat-inactivated fetal calf serum (FCS; GIBCO Invitrogen) and 20 ng/ml M-CSF (Peprotech) for 3 days. On day 3, unattached cells were collected, and the monocyte phenotype was confirmed by flow cytometry as CD45⁺CD11b⁺CD115⁺ cells. The cells were re-suspended in medium and seeded at a density of 1×10⁵ cells/ml. Extracellular S100A9 (20 μg/mL) with or without the S100A9 antagonist ABR-238901 at a concentration of 100 μM were incubated in the presence of 10 μM ZnCl₂ (Sigma) and 2 mM CaCl₂ for 15 minutes and added to the monocyte culture. Recombinant human endotoxin-free S100A9 protein was provided by Active Biotech AB. Following 24 h stimulation, intracellular Nur77 expression was analysed by flow cytometry.

### Nur77 Activity Measurement by Luciferase Assay

Low-passage RAW264.7 mouse macrophages (ATCC #TIB-71) were seeded at 125.000 cells/mL in DMEM medium containing 2 mM L-glutamine (Gibco), 10% Fetal Calf Serum (Gibco), and 1% penicillin/streptomycin (Invitrogen). The next day, cells were transfected with pXP1-NurRE-fluc or pGL2-4xNBRE-fluc firefly luciferase Nur77 reporter constructs using Lipofectamine LTX with PLUS reagent (Invitrogen) according to manufacturer's instructions. pRluc-N3, which constitutively expresses Renilla luciferase from the CMV promoter, was co-transfected as an internal control to verify transfection efficiency. Extracellular S100A9 (20 μg/mL) was incubated in the presence of 10 μM ZnCl₂ (Sigma) for 15 minutes with or without 100 μM of ABR-238901, and added to the cells 4 hours after transfection. After 16 hours of stimulation, the cells were washed and firefly and Renilla luciferase activities were determined using the Dual-Luciferase Reporter Assay System and a GloMax luminescent plate reader (both Promega).

### Results

In order to examine whether acute extracellular S100A9 release has a direct pathogenic role in the development of post-MI heart failure (HF), we tested whether S100A9 blockade during the acute inflammatory post-MI phase improves cardiac recovery and function. We induced MI by permanent left coronary artery ligation in wild-type C57BL/6 mice, and treated the mice for the first 3 days post-MI with the specific S100A9 blocker ABR-238901 that blocks the interaction between extracellular S100A9 and both its cell-surface receptors, TLR4 and RAGE (FIG. 7). A dose of 30 mg/Kg ABR-238901 (ABR) was administered as daily i.p. injections in PBS at 0 h, 24 h and 48 h after left coronary artery ligation. Sham-operated mice and mice with induced MI treated with PBS were used as controls. Cardiac function was evaluated by echocardiography at baseline and at 3, 7, and 21 days after MI (FIG. 1A). Left ventricular EF was higher throughout the follow-up period in mice receiving S100A9 blockade, reaching an average of 36% by day 21 compared with 17% in PBS-treated controls (FIG. 1B, P=0.021). On day 21, we also found a tendency towards smaller average myocardial scar size in ABR-treated mice compared to controls (FIG. 1C). In order to confirm these initial results and to evaluate the hemodynamic effect of the treatment, we repeated the experiment using cardiac magnetic resonance imaging (MR) to evaluate left ventricular EF, EDV, ESV, and cardiac output (CO). In this experiment, left ventricular EF was significantly higher on day 7 (25% vs 16%, P<0.01) and on day 21 (26% vs 12%, P<0.001) in mice receiving S100A9 blockade compared to PBS-treated MI controls (FIG. 1D-E). While EF remained constant from day 3 to day 21 in the ABR-treated group (29% vs 26%, P=n.s.), the control mice suffered progressive deterioration of left ventricular systolic function (24% vs 12%, P<0.001), confirming a long-lasting effect of S100A9 blockade beyond the 3-day treatment period. We recorded a remarkable hemodynamic recovery from day 3 to day 21 in the ABR-treated mice, with an approximately 30% increase in cardiac output (CO), from 8.1 mL/min on day 3 to 10.4 mL/min on day 21 (FIG. 1F). An opposite trend was observed in the PBS-treated control group (FIG. 1F). Cardiac remodeling, characterized by left ventricular dilation, occurred in both MI groups (FIG. 1G-H). There was a tendency towards lower EDV and ESV in the ABR-treated mice, but the differences between the groups did not reach statistical significance.

In a separate experiment, we investigated whether S100A9 blockade leads to reduced MI size. Following 3 days treatment with ABR-238901 or PBS, different mouse groups were sacrificed on days 3 and 7 post-MI. The ischemic area at risk (AAR) was identified by trans-apical Evans Blue injection at harvest, and the salvaged viable myocardium by the triphenyl tetrazolium chloride (TTC) reaction. We found that the size of the infarction, expressed as percentage of the AAR volume, was smaller on both days 3 and 7 post-MI (FIG. 11) in mice receiving S100A9 blockade (n=5-7 mice/group). Additionally, the treatment significantly reduced the percentage of apoptotic cells in the myocardium, identified by the TUNEL method (FIG. 1J).

### Conclusion

It was demonstrated that short-term treatment with ABR-238901 during the acute inflammatory post-MI period is advantageous compared to the control group, leading to better heart function and hemodynamic parameters, smaller infarction size and reduced apoptosis in the myocardium.

### Example 3: S100A9 Blockade Impairs Myeloid Cell Production and Egression from the Bone Marrow and Reduces the Presence of Neutrophils and Macrophages in the Infarcted Myocardium

### Materials and Methods

### Flow Cytometry

Immune cells from blood, spleen, heart and bone-marrow were collected at 2 or 7 days post-MI, and analysed by flow-cytometry. Mice were anesthetized by intraperitoneal injection of a mixture of xylazine (10 mg/kg) and ketamine (100 mg/kg). After 10 minutes, blood was collected by cardiac punction. Thereafter, the spleen was removed and the heart was perfused with 5 ml ice-cold PBS. Perfused hearts were excised and collected into tubes with PBS and kept on ice. Femurs and tibias from each mouse were used to obtain whole bone marrow for analysis. Blood was stained with fluorochrome-labelled antibodies against mouse monocyte and neutrophil markers diluted in MACS buffer (PBS, 10% FCS and 0.5% BSA), followed by treatment with erythrocyte lysis buffer, washing and analysis. Spleens were mashed trough a cell strainer in ice-cold PBS. Hearts were cut into small pieces and placed into tubes containing 2.5 ml digestion cocktail (Collagenase I, 450 U/ml; Collagenase XI, 125 U/ml; DNAase I, 60 U/ml; Hyaluronidase type 1-5, 60 U/ml diluted in PBS containing Ca and Mg) incubated for 1 h at 37° C. with shaking at 220 rpm. Bone marrow was flushed from femur and tibia using ice-cold PBS. Cell suspensions from spleen, heart and bone marrow were filtered through a 40 um cell strainer (BD), treated with erythrocyte lysis buffer, washed and stained. For intracellular staining, cells were fixed, permeabilized and stained. Cell populations were analyzed by flow cytometry in a Gallios Flow Cytometer (Backman Coulter), and the results were analyzed using FlowJo (FlowJo®, LLC; Tree Star). The following anti-mouse antibodies were used for the flow cytometric analysis: anti-CD45 (30-F11), anti-CD11b (M1/70), anti-Ly6C (HK1.4), anti-CD115 (AFS98), anti-Ly6G (1A8), anti-F4/80 (BM8), anti-CCR2 (475301), anti-Ter119 (TER119), anti-Gr1 (RB6-8C5), anti-CD45R/B220 (RA3-6B2), anti-CD3ξ (145-2C11), anti-NK1.1 (PK136), anti-Ly6A/E (Sca-1) (E13-1617), anti-CD117 (c-kit) (2B8), anti-CD48 (HM48.1), anti-CD150/SLAM (TC15-12F12.2), anti-CD16/32 (93), anti-CD34 (MEC14.7) (all from BioLegend, San Diego, Calif., USA); anti-MerTK (DSSMMER) and anti-Nur77 (12.14) (eBioscience).

### Proliferation Analysis by BrdU Incorporation

Mice were injected intraperitoneally with 1 mg BrdU 24 hours prior to organ harvest. Intracellular BrdU staining was performed in hematopoietic stem cells collected from the bone marrow using BrdU flow kits (BD Pharmigen™), according to the manufacturer instructions. BrdU expression was analysed by flow cytometry.

Other procedures were carried out as outlined in Example 2.

### Results

Neutrophilia and monocytosis, elevated circulating numbers of neutrophils and monocytes, have been associated with an adverse prognosis in MI patients. We studied the effects of S100A9 blockade on myeloid cell populations in blood, heart, and myeloid organs on day 2 post-MI, at the peak of the acute inflammatory period. The gating strategy is described in FIG. 8. We found that S100A9 blockade significantly lowered the numbers of neutrophils in blood and myocardium (FIG. 2A,H). In contrast, neutrophil counts in the bone marrow were highly increased compared to the MI control group (FIG. 2C). This effect could not be explained by increased production of neutrophils in the bone marrow, as ABR-238901 also reduced the numbers of proliferating BrdU⁺ Lin⁻Sca⁺Kit⁺CD150⁺CD48⁻ hematopoietic stem cells (HSC), measured at 24 h after i.v. BrdU administration (FIG. 2G). Taken together, these data suggest that the reduced presence of neutrophils in the circulation and in the heart in mice receiving S100A9 blockade is due to a combination of decreased production and impaired cell egression from the bone marrow. We also found significantly higher numbers of monocytes in the bone marrow (FIG. 2F; FIG. 9D), and lower numbers of macrophages in the heart of ABR-238901-treated mice (FIG. 2J), suggesting that the treatment has similar effects on monocyte/macrophage dynamics. S100A9 blockade did not have a significant effect on the numbers of neutrophils and monocytes in the spleen at this stage (FIG. 2B, E; FIG. 9C).

### Conclusion

It was demonstrated that S100A9 blockade impairs myeloid cell production and egression from the bone marrow and reduces the presence of pro-inflammatory neutrophils and macrophages in blood and in the infarcted myocardium.

### Example 4: Short-Term S100A9 Blockade Inhibits Inflammation and Favours a Reparatory Environment in the Ischemic Myocardium

### Materials and Methods

### Quantitative PCR

Total RNA was isolated from whole heart tissue using the TRI Reagent™ Solution (Invitrogen) according to the manufacturer's instructions. cDNA was generated from 2 μg of total RNA per sample using the High Capacity RNA to cDNA™ Kit (Applied Biosystems™). Quantitative real-time TaqMan PCR was performed using the following TaqMan primers (Applied Biosystems): Il-1b (Mm00434228_m1), Arg-1 (Mm00475988_m1), Ym-1 (Mm00657889_mH), Cc/5 (Mm01302427_m1), Tnf-α (Mm00443258_m1), Il-12 (Mm01288989_m1), IFN-γ (Mm01168134_m1), Il-10 (Mm00439614_m1), TGF-β (Mm01178820_m1), Il-4 (Mm00445259_m1) and housekeeping gene Hypoxantine-guanine phosphoribosyltransferase (HPRT) (Mm01324427_m1). Quantitative PCR was run on a ViiA7 Real-Time PCR System (Applied Biosystems) and data were quantified with the 2^{-ΔCt} method.

### Results

In order to assess the effects of the treatment on the post-MI cardiac environment, we performed qPCR analysis of mRNA encoding for proteins involved in myocardial inflammation and repair. The analysis was done in whole heart tissue collected at 3 days post-MI from mice receiving ABR-238901 or PBS (n=5 mice per group). Compared to MI controls, the treatment inhibited the expression of mRNA encoding for the pro-inflammatory cytokines IL-12 and IFN-γ, and enhanced expression of Arg-1, Ym-1 and IL-4 mRNA, signature genes of a reparatory macrophage phenotype (FIG. 3A, B, E, F, G, K). The expression of IL-1β, TNFα, IL-10 and TGFβ was not affected at this stage (FIG. 3C, D, H). Gene expression of the chemokine CCL5/RANTES, previously shown to play a central role in deleterious post-MI inflammation responses, was also inhibited by S100A9 blockade (FIG. 3J). Interestingly, ABR-238901 also reduced plasma levels of S100A8/A9 (FIG. 3L) and S100A8/A9 presence in the infarcted myocardium (FIG. 3M) at 3 days post-MI. As neutrophils are the main producers of S100A8/A9, this effect is in line with the blunted local and systemic neutrophil response to MI observed in ABR-238901-treated mice (FIG. 2A, H).

### Conclusion

It has been demonstrated that short-term S100A9 blockade inhibits inflammatory gene expression and favours a reparatory environment in the ischemic myocardium.

### Example 5: Long-Term S100A9 Blockade Impacts Myocardial Repair, Leading to Cardiac Remodeling and Impaired Function

### Materials and Methods

MI induction and other procedures were carried out as outlined in Example 2. For long-term experiments of up to 21 days, a daily dose of 30 mg/Kg ABR-238901 was diluted in PBS and administered by daily i.p. injections for the first 3 days post-MI until the mice recovered and started to drink normally, and thereafter p.o. ad-libitum in meglumine buffer (FIG. 4A). Adequate treatment dosage was ensured by closely monitoring daily fluid intake.

### Results

Considering the beneficial effects of short-term S100A9 blockade on cardiac inflammation and function post-MI, we asked if we could achieve improved therapeutic results by continuing the treatment long-term. To this end, we continued the ABR-238901 treatment for 21 days after permanent left coronary artery ligation. The control MI mice were treated with PBS (FIG. 4A). In contrast to the short-term S100A9 blockade, extending the treatment throughout the repair and recovery phase of the immune response to MI induced progressive deterioration of cardiac function (FIG. 4B). Myocardial remodeling was also accelerated in ABR-238901-treated mice, leading to increased EDV and ESV on day 21 compared with controls (FIG. 4C-D). The size of the myocardial scar at 21 days was also increased (FIG. 4E).

In an attempt to explain these intriguing results, we assessed the effects of the extended treatment on the presence of myeloid cells in blood, spleen, bone marrow and myocardium, with particular emphasis on cellular populations involved in myocardial repair. The cellular infiltration was analysed by flow cytometry on day 7, at the peak of the reparatory post-MI period. We found significantly lower numbers of circulating neutrophils and monocytes in mice receiving S100A9 blockade for 7 days (FIG. 4F, I; FIG. 10A). Conversely, the treatment induced monocyte accumulation in the spleen compared to the MI controls (FIG. 4J). There were no differences in cell counts in the bone marrow at this stage (FIG. 4H, K). Similar to day 3, the numbers of neutrophils in the spleen were not significantly affected (FIG. 4G). Consequent to the impaired monocyte dynamics, the presence of monocytes and total CD11b⁺F4/80⁺ macrophages in the heart was potently reduced by the treatment (FIG. 4m, n). Reparatory macrophages in the ischemic myocardium have previously been defined as CD11b⁺F4/80⁺Ly6C^{lo} cells. Further, the efferocytosis receptor myeloid-epithelial-reproductive receptor tyrosine kinase (MerTK), predominantly expressed on Ly6C^{lo} macrophages, has recently been found to play a major role in cardiac repair post-MI. In order to investigate the possible mechanisms leading to impaired cardiac function in mice receiving long-term S100A9 blockade, we examined the presence and function of Ly6C^{lo}MerTK^{hi} macrophages in the myocardium at 7 days post-MI. We found an approximately 50% reduction of the number of reparatory macrophages in hearts of mice receiving ABR-238901 compared to controls (FIG. 40). Moreover, the percentage of reparatory Ly6C^{lo}MerTK^{hi} out of total CD11b⁺F4/80⁺ macrophages was significantly decreased in these mice, suggestive of impaired phenotype switch of monocytes towards reparatory macrophages (FIG. 4P). In order to test the functional importance of these findings, we used α-MHC-mCherry mice expressing cardiac-specific red fluorescent Cherry protein under the mouse a-myosin heavy-chain promoter. Following MI, the number of macrophages having phagocytosed mCherry+cardiomyocytes were also reduced by approximately 50% (FIG. 4Q). mCherry levels were much higher in MerTK^{hi} compared to MerTK^{lo} macrophages, supporting the role of this macrophage subpopulation as dominant effectors of efferocytosis (FIG. 4R). S100A9 blockade did not affect mCherry mean fluorescent intensity (MFI) in either sub-population (FIG. 4R), suggesting that the inhibition of efferocytosis is due to reduced presence of reparatory macrophages in the myocardium, rather than to impaired MerTK function.

Expression of the transcription factor Nur77 in Ly6C^{hi} monocytes is critical for the generation of the non-classical or patrolling Ly6C^{lo} monocytes under homeostatic conditions, and has recently been demonstrated to play a major role in the differentiation of Ly6C^{hi} monocytes into reparatory F4/80+Ly6C^{lo} macrophages in the post-ischemic myocardium. In order to investigate the underlying molecular mechanisms involved in the inhibition of myocardial repair by S100A9 blockade, we induced MI in C57Bl/6 mice transplanted with Nur77-EGFP transgenic bone marrow expressing the fluorescent EGFP protein under the Nur77 promoter (FIG. 4S). The expression of Nur77 was measured by flow cytometry in blood monocytes and heart macrophages harvested on day 7 post-MI from mice receiving 7 days ABR-238901 treatment or PBS. We found that blockade of extracellular S100A9 led to lower intracellular Nur77 levels in blood Ly6C^{hi/int} monocytes (FIG. 4T) and reduced the numbers of reparatory Ly6C^{lo}MerTK^{hi} in the myocardium (FIG. 11A). Nur77 levels were unaffected in the fully-differentiated Ly6C^{lo} monocytes and Ly6C^{lo}MerTK^{hi} macrophages (FIG. 4t; FIG. 11b). Further, we studied the influence of S100A9 on Nur77 expression and activity in monocytes and macrophages in-vitro, and the ability of ABR-238901 to inhibit these effects. Stimulation with recombinant extracellular S100A9 for 24 h increased Nur77 expression in bone marrow-derived C57BL/6 monocytes (FIG. 4U). S100A9 also increased Nur77 activity in RAW264.7 macrophages transfected with firefly luciferase constructs coupled to the specific Nur77 response elements NBRE and NurRE (FIG. 4V). Both of these effects were inhibited by ABR-238901 (FIG. 4U-V). Taken together, these data reveal a stimulatory role of extracellular S100A9 on Nur77 expression and function in monocytes and macrophages, and provide mechanistic evidence for the impaired development of reparatory macrophages in the ischemic myocardium induced by the long-term ABR-238901 treatment.

As we found that short-term and long-term S100A9 blockade have opposite effects on post-MI cardiac function and repair, we directly compared the effects of the two treatment strategies on the presence of reparatory Ly6C^{lo}MerTK^{hi} macrophages in the myocardium. Reparatory macrophage numbers were analysed in hearts collected at 7 days post-MI from mice with induced MI treated with ABR-238901 for 3 or 7 days. In mice receiving 3 days ABR-238901 treatment, the intra-myocardial counts of reparatory macrophages were on par with the PBS-treated MI controls, whereas mice receiving continuous S100A9 blockade for 7 days had significantly lower levels of these cells in the heart (FIG. 4W). These findings suggest that, in contrast to the long-term treatment, short-term inhibition of S100A9 function does not impact cardiac repair, explaining the long-term persistence of the beneficial effects of the treatment.

### Conclusion

It was demonstrated that long-term S100A9 blockade negatively impacts myocardial repair, leading to increased cardiac remodelling and impaired function by inhibiting the activation of the transcription factor Nur77 and reducing the presence of reparatory macrophages in the post-ischemic myocardium.

### Example 6: Short-Term S100A9 Blockade Efficiently Improves Cardiac Function and Hemodynamic Parameters after Myocardial Ischemia/Reperfusion

### Materials and Methods

Procedures were carried out as outlined in Example 2. In contrast to permanent ischemia, the coronary artery occlusion was maintained for 60 minutes followed by removal of the ligature, allowing for reperfusion of the ischemic myocardium. Three doses of 30 mg/Kg ABR-238901 diluted in PBS were administered by daily i.p. injections at the time of the reperfusion, and after 24 h and 48 h (FIG. 5A).

### Results

As shown in FIG. 1, short-term ABR-238901 treatment improves cardiac function after permanent coronary ischemia associated with large transmural infarctions. However, the current clinical practice involves rapid coronary revascularization in MI, the most effective method to reduce myocardial damage and improve function. Thus, in order for a novel treatment to be clinically relevant, it has to be able to improve cardiac function on top of the beneficial effects of reperfusion. In order to mimic the clinical scenario, we induced temporary ischemia by coronary artery ligation for 60 minutes in C57BL/6 mice, followed by revascularization. The mice were treated with ABR-238901 or PBS i.p. for 3 days, and cardiac function was assessed at 3, 7, 14, and 21 days post-MI (FIG. 5A). The left ventricular ejection fraction was higher in the PBS-treated MI control mice in this experiment, reaching an average of 34% by day 21 (FIG. 5B), compared to 17% after permanent ischemia (FIG. 1B), confirming the efficiency of coronary revascularization in the model. Compared to the MI control group, 3 days S100A9 blockade led to highly improved left ventricular EF already at the end of the first week post-MI, reaching an average of 45% in ABR-238901 treated mice vs. 35% in controls on day 7 (FIG. 5B, P=0.006). In contrast to the control group, EF continued to improve in ABR-238901-treated mice from day 3 to day 21, increasing from 42% to 48%. No such improvement has been recorded in PBS-treated MI mice. Cardiac output was also significantly higher in mice receiving S100A9 blockade, reaching an average of 15.7 mL/min by day 21, compared to 11.1 mL/min in the untreated group (FIG. 5C, P=0.002). There were no significant differences in average ESV and EDV between the groups, although ESV remained lower in ABR-238901-treated mice at all time points (FIG. 5D-E). Similar to permanent ischemia, the treatment increased the numbers of neutrophils and monocytes in the bone marrow and decreased macrophage infiltration in the myocardium following myocardial ischemia/reperfusion (FIG. 5F, G, J).

### Conclusion

Our data reveal that short-term S100A9 blockade efficiently improves cardiac function and hemodynamic parameters after myocardial ischemia/reperfusion in mice.

### Example 7: S100A9 Blockade with ABR-238901 Improves Cardiac Function in Sepsis

### Materials and Methods

### Animal Models and Treatment

Female C57BL/6NRJ mice, 10 weeks of age, were purchased from Janvier Labs and housed under 12 h:12 h light:dark cycles with access to food and water ad libitum. The mice were randomly assigned to the two treatment groups, n=5 mice per group. Sepsis was induced by intraperitoneal (i.p.) injection of 5 mg/kg bacterial lipopolysaccharide (Sigma-Aldrich) in all animals. Immediately thereafter and 6 h later, the mice received treatment with 30 mg/kg ABR-238901 or an equivalent volume of PBS administered i.p. The mice were monitored daily to ensure their well-being. All mouse studies were approved by the Animal Research Ethics Committee of Lund University.

### Mouse Echocardiography

Transthoracic echocardiography was performed at baseline before induction of sepsis, and at 6 h, 12 h and 24 h using the Vevo 3100 platform (VisualSonics) equipped with a MX550D imaging transducer. Anesthesia was induced with 2% Isoflurane (AbbVie) and maintained with 1.25% Isoflurane throughout imaging. Hair was removed using depilatory cream before the application of pre-warmed ultrasound gel. Parasternal long axis recordings were performed in the plane containing the aortic valves and a maximal apex view. Analysis was carried out using the VevoLab software (Visual Sonics). Briefly, the perimeter and long axis of the left ventricle were traced at the end of diastole and at the end of systole and used to calculate left ventricular end-systolic and end-diastolic volumes. Left ventricular stroke volume was calculated as the difference between the left ventricular end-diastolic and end-systolic volumes. Left ventricular ejection fraction was calculated as percentage stroke volume out of the end-diastolic volume. Cardiac output was calculated by multiplying the stroke volume with the heart rate at the time of the examination.

### Results

The experimental layout is presented in FIG. 13A. We investigated the effects of S100A9 blockade with ABR-238901 on cardiac function and hemodynamic parameters in sepsis. Left ventricular ejection fraction (LVEF) at 12 h after induction of sepsis was significantly higher in mice treated with ABR-238901 compared with mice treated with PBS (44.7% vs 33.2%, P=0.015, FIG. 13B). At the 24 h time point, left ventricular ejection fraction returned to baseline levels in mice treated with ABR-238901, while mice treated with PBS maintained a severely reduced LVEF (54.74% vs 36.72%, P=0.0008, FIG. 13B). Cardiac output was also improved at 24 h in mice with sepsis treated with ABR-238901 (9.17 mL/min vs 6.66 mL/min, P=0.064, FIG. 13C).

### Conclusion

Inhibition of S100A9 with ABR-238901 reverses sepsis-induced cardiomyopathy and rescues cardiac function as measured by left ventricular ejection fraction and cardiac output.

### Example 8: S100A9 Blockade with ABR-238901 Reduces Mortality in Sepsis

### Materials and Methods

### Animal Models and Treatment

General animal care, induction of sepsis and treatments were performed as described under Example 7. We initially included a total of 10 mice per treatment group, pooled from two independent experiments. To assess mortality after induction of sepsis, the mice were monitored at least three times a day for 7 days. One mouse in the group treated with ABR-238901 did not complete the follow-up and was excluded from the experiment due to a higher percentage of weight loss during follow-up than the 20% allowed by the ethical permit. The final group size was n=9 mice in the ABR-238901-treated group, and n=10 in the PBS-treated group. The experimental layout is outlined in FIG. 14A. Mice showing clinical signs of terminal illness that fulfilled the pre-specified termination points specified in the ethical permit awarded by the Lund University Ethics Committee were euthanized. Weight loss more than 20% is not considered a clinical sign of terminal illness, but was defined by the Ethical Committee as an experiment termination criterion.

### Results

We investigated the effects of S100A9 blockade with ABR-238901 on animal mortality across a 7-day period following induction of sepsis (FIG. 14A). Animal survival in mice with sepsis treated with ABR-238901 was 100%, compared with 60% in mice with sepsis treated with PBS (P=0.038, FIG. 14B).

### Conclusion

S100A9 blockade by ABR-238901 treatment reverses the lethal effects of sepsis and improves survival.

### Example 9: S100A9 Blockade with ABR-238901 Ameliorates Weight Loss During Sepsis

### Materials and Methods

### Animal Models and Treatment

General animal care, induction of sepsis and treatments were performed as described under Example 7. We included 5 mice per treatment group. After induction of sepsis, the mice were monitored for 7 days (FIG. 15A). To evaluate bodyweight, mice were weighed daily at the same time points during the course of the experiment.

### Results

Weight loss occurs during sepsis due to increased catabolism and dehydration, and is correlated with increased mortality in animal models. Here, we investigated the effects of ABR-238901 treatment on weight loss during the acute period in an animal model of sepsis. The experimental layout is described in FIG. 15A. Septic mice treated with ABR-238901 lost significantly less weight compared to the PBS-treated controls during the first 4 days after sepsis induction. One day after induction of sepsis, ABR-238901-treated mice had a significantly lower decrease in body weight (5.2% vs 10.3%, P=0.0007), which persisted throughout days 2-4, as follows: day 2 (11.7% vs 17.7%, P=0.0145), day 3 (8.8% vs 16.6%, P=0.00002), day 4 (4% vs 8%, P=0.034).

### Conclusion

S100A9 blockade by treatment with ABR-238901 significantly prevents weight loss during the acute period of sepsis.

### Example 10: S100A9 Blockade with ABR-238901 Lowers Plasma Concentration of Pro-Inflammatory Mediators in Sepsis

### Materials and Methods

### Animal Models and Treatment

General animal care, induction of sepsis and treatments were performed as described under Example 7. We included 5 mice per treatment group. The mice were sacrificed 24 h after sepsis induction and blood was drawn using cardiac puncture with an EDTA 0.5M coated syringe. Blood was spun down at 3000 rpm for 10 minutes and plasma was collected.

### Cytokine Measurements

Cytokine measurements were performed using the Cytokine & Chemokine 36-Plex Mouse procartaPlex™ P1A (ThermoFischer Scientific) as per manufacturer's instructions. Data are presented as mean±SD.

### Results

Sepsis involves a widespread activation of the immune system, characterized by activation of inflammatory cells and subsequent release of pro-inflammatory mediators into the bloodstream. These mediators induce organ dysfunction, including cardiac inflammation and septic cardiomyopathy, weight loss, and mortality. We investigated the effects of S100A9 inhibition with ABR-238901 on the levels of cytokines, chemokines and growth factors in plasma during the acute period of sepsis. The experimental layout is outlined in FIG. 16A. ABR-238901 treatment decreased plasma levels of a large number of molecules involved in the immune response (IL-1b, P=0.031; IL-6, P=0.037; IL17-A, P=0.02; IL-22, P=0.0092; IFN-γ, P=0.03; MCP-1, P=0.04; LIX, P=0.01; LIF, P=0.03; GM-CSF, P=0.04).

### Conclusion

Treatment with ABR-238901 inhibits several immune and inflammatory pathways and lowers the systemic inflammatory burden associated with sepsis. This can explain the beneficial effects of the treatment on cardiac function, mortality and weight loss demonstrated in Examples 7-9.

### Example 11: S100A9 Blockade with ABR-238901 Improves Cardiac Function, Mortality, Weight Loss and Systemic Inflammation in Sepsis, Regardless of the Causal Bacteria

Cardiac dysfunction, mortality, weight loss and systemic inflammation in sepsis are due to an exaggerated immune response to a bacterial infection, rather than to the bacteria themselves. Accordingly, the beneficial effects of S100A9 blockade with ABR-238901 in sepsis, as demonstrated in Examples 7-10, are considered to present regardless of the original bacteria type that causes the septic reaction.

### Materials and Methods

### Animal Models and Treatment

Sepsis is induced in C57BL/6NRJ mice or other mouse strains by intraperitoneal (i.p.), intravenous (i.v.), intranasal, or subcutaneous (s.c.) injection of different types of live bacteria. Alternatively, sepsis is induced by caecum ligation and puncture, resulting in peritonitis and sepsis due to intestinal bacteria. I.p. treatment with a compound for formula (I), for example ABR-238901, is administered during the acute phase of sepsis as described in Examples 7-10, with the potential addition of additional time points, depending on the dynamics of the disease. PBS-treated mice are used as controls.

### Analysis

Mouse echocardiography, mortality, weight loss and inflammatory activation will be assessed as described under Examples 7-10.

### Results

It is expected that treatment with ABR-238901 or another compound of formula (I) inhibits sepsis-induced myocardial and systemic inflammation, reduces septic cardiomyopathy and improves cardiac function, as well as prevents mortality and weight loss in all sepsis models, mediated by live bacteria or by bacterial products.

### Conclusion

It is envisioned that S100A9 blockade with ABR-238901 or another compound of formula (I), mainly targeting the extracellular form of the protein, during the acute phase of the disease inhibits inflammation and improves cardiac function and survival in sepsis, regardless of the initial triggering factor of the disease.

### Example 12: S100A9 Blockade with ABR-238901 Improves Cardiac Function in Myocarditis

### Materials and Methods

### Animal Models and Induction of Myocarditis

Female BALB/c mice, 8 weeks of age, were purchased from Taconic and housed under 12 h:12 h light:dark cycles with access to food and water ad libitum. To induce myocarditis, all mice were injected subcutaneously (s.c.) with 100 μg murine cardiac a-myosin heavy chain peptide (Ac-RSLKLMATLFSTYASADR-OH, Anaspec) emulsified 1:1 in complete Freund's adjuvant containing 1 mg/mL heat-killed Mycobacterium tuberculosis H37Ra (Sigma-Aldrich), on days 0 and 7 (FIG. 17A). ABR-238901 was administered orally ad-libitum diluted in meglumine buffer (to prevent precipitation in the drinking bottle during the course of the experiment) at a dose of 30 mg/kg/day, starting on day 7 and throughout the course of the experiment (n=9 mice). Fluid intake was closely monitored during the experiment to ensure an appropriate drug dosage. Mice with induced myocarditis drinking meglumine buffer alone ad-libitum were used as controls (n=12 mice). Mice received daily attendance to ensure their well-being. All mouse studies were approved by the Animal Research Ethics Committee of Lund University.

### Mouse Echocardiography

Transthoracic echocardiography was performed as described under Example 7. Cardiac function was evaluated by echocardiography at baseline and at 21, 28 and 35 days after the first injection of murine cardiac a-myosin heavy chain peptide.

### Results

The immune cell-mediated cardiac inflammation characteristic for myocarditis leads to myocardial injury and progressive loss of function. We investigated the effects of S100A9 blockade with ABR-238901 on cardiac function during the acute phase of myocarditis. The experimental layout is presented in FIG. 17A. Cardiac function, measured by left ventricular ejection fraction (LVEF), was significantly improved by the ABR-238901 treatment compared with mice receiving meglumine buffer alone, as measured on days 28 (52% vs 47%, P=0.013) and day 35 (53% vs 46%; P=0.035) (FIG. 17B). Cardiac output of mice treated with ABR-238901 was also significantly higher compared with the meglumine controls on days 28 (11.26 vs 9.36 mL/min; P=0.027) and day 35 (12.03 vs 9.61 mL/min; P=0.034) (FIG. 17C).

### Conclusion

S100A9 blockade with ABR-238901 during the acute phase of the disease improves left ventricular function and hemodynamic parameters in myocarditis.

### Example 13: S100A9 Blockade with ABR-238901 Inhibits the Infiltration of Inflammatory Cells in the Heart and Heart-Draining Lymph Nodes During Myocarditis

### Materials and Methods

### Animal Models and Treatment

Animal care, induction of myocarditis and treatments were performed as described above (Example 11) (n=10 mice per group). The mice were sacrificed on day 21, and hearts and mediastinal heart-draining lymph nodes were isolated.

### Flow Cytometry

Single cell suspensions were obtained from the organs by mechanic sectioning and enzymatic digestion, and the cells were stained with fluorescently-conjugated antibodies for analysis by flow cytometry. After euthanization, the mice were perfused intra-cardially with 20 ml ice-cold PBS, hearts and mediastinal lymph nodes were harvested and placed in PBS on ice. Hearts were cut in small pieces for 2 min while placed in digestion mixture containing 450 U/ml Collagenase I-S (Sigma-Aldrich, C0130), 125 U/ml Collagenase XI (Sigma-Aldrich, C7657), 60 U/ml Hyaluronidase Type I-S(Sigma-Aldrich H3506), 60 U/ml DNAse I (Sigma-Aldrich, DN25), and 20 mM HEPES (Fischer Scientific, SH3023701) in PBS, and thereafter incubated for 1 h at 37° C. with gentle agitation. Enzymatically-digested tissues were passed through a 70 μm cell strainer and washed twice with PBS. Mediastinal lymph nodes were passed through a 70 μm cell strainer and washed twice with PBS before staining. Single cell suspensions were stained in PBS with Zombie Aqua live/dead (BioLegend, 423102) for 10 min at RT followed by extracellular staining in FC-buffer (PBS, 0.5% BSA and 20 mM EDTA) with fluorescently-conjugated antibodies for 20 min on ice. The following monoclonal antibodies were used: anti-CD3ε (clone 145-2C11), anti-CD4 (clone OKT4), anti-CD8 (clone RPA-T8), anti-CD11b (clone EP1345Y), anti-CD11c (clone N418), anti-CD19 (clone 6D5), anti-CD44 (clone IM7), anti-CD45.2 (clone 104), anti-CD49b (clone DX5) anti-CD62L (clone MEL-14), anti-CD64 (clone), anti-CD69 (clone X54-5/7.1), anti-CD115 (clone AFS98), anti-Ly-6C (clone HK1.4), anti-Ly-6G (clone 1A8), anti-MerTK (clone DS5MMER), anti-MHCII (clone M5/114.15.2), anti-Siglec-F (clone E50-2440). Appropriate single stains were performed using AbC Total Antibody Compensation Bead kit (Thermo Fischer Scientific) or with cells for live/dead. Flow cytometry analysis was done using a Gallios Flow Cytometer (Beckman Coulter) and data was analyzed using the FlowJo software (Tree Star).

### Results

Inflammatory infiltrates in the myocardium are the main drivers of myocarditis. The heart-draining lymph nodes in the mediastinum are the main location for priming of the pathogenic CD4+ and CD8+ effector T cells through myocardial antigen presentation by conventional dendritic cells (cDCs). Eosinophils have also been shown to play an important pathogenic role in myocarditis. Here, we investigated the effects of S100A9 inhibition with ABR-238901 during the acute phase of myocarditis on the cardiac infiltration of DCs and eosinophils, and on the presence of pathogenic T cells in the cardiac-draining lymph nodes. The experimental layout is presented in FIG. 18A. On day 21 after induction of myocarditis, the hearts of mice treated with ABR-238901 had decreased infiltration of cDCs (expressed as % of live CD45⁺ leukocytes) compared with the group with myocarditis receiving meglumine buffer alone (5.7 vs 8.9%, P=0.008, n=10 mice per group, FIG. 18B). Cardiac eosinophil infiltration was also reduced in the ABR-238901-treated group, also indicative of a lower degree of inflammation (7.5 vs 12.3%, P=0.03, FIG. 18C). In the heart-draining lymph nodes, the mice treated with ABR-238901 showed a trend of reduced presence of total CD3⁺ T cells proportion expressed as % of live CD45⁺ leukocytes (59.5 vs 71.9%, P=0.0518, FIG. 18D). Further, we found a markedly lower proportion of effector memory (CD44⁺CD62L⁻) CD4⁺ (15.9 vs 33.7%, P<0.0001, FIG. 18E) and CD8⁺ (23.3 vs 55.3%, P=0.0001, FIG. 18F) T cells in mice with myocarditis treated with ABR-238901 compared with controls.

### Conclusion

S100A9 blockade by ABR-238901 treatment during the acute phase of the disease significantly inhibits the inflammatory activation in myocarditis, as witnessed by lower infiltration of antigen-presenting cDCs and eosinophils in the heart, and reduced presence of pathogenic CD4⁺ and CD8⁺ effector T cell populations in the heart-draining lymph nodes.

### Example 14: S100A9 Blockade with ABR-238901 Inhibits the Infiltration of Inflammatory Cells in the Heart and Heart-Draining Lymph Nodes, and Improves Cardiac Function in Myocarditis Regardless of the Initial Trigger of the Disease

In myocarditis, myocardial damage is mediated by an immune and inflammatory attack against the myocardium, triggered, among others, by viruses, bacteria, autoantibodies or activated leukocytes. Myocarditis also occurs in systemic autoinflammatory diseases such as systemic lupus erythematosus, rheumatoid arthritis or sarcoidosis. Since S100A9 plays a central role in the activation of the innate immune response, which eventually leads to subsequent activation of the adaptive immune response, it is envisioned that S100A9 blockade with ABR-238901 effectively inhibits the immune and inflammatory activation, myocardial damage and cardiac dysfunction in myocarditis, regardless of the original trigger.

### Materials and Methods

### Animal Models and Treatment

Viral myocarditis is induced in BALC/c mice or other sensitive mouse strains by intraperitoneal (i.p.) injection of live Coxackievirus B3 or other viruses (103 plaque-forming units in 0.1 mL PBS). Dendritic cell (DC)-mediated myocarditis is induced by i.p. injection of three doses (day 0, 2 and 4) of 3-6×10⁵ DCs that have been antigen-loaded and pre-activated by incubation with murine cardiac a-myosin heavy chain peptide, LPS and anti-CD40 antibodies. T cell-mediated myocarditis is induced in immunodeficient Scid mice by i.p. or intravenous (i.v.) injection of 10⁷ T cells that have previously been primed and activated by antigen-presenting cells in the presence of murine cardiac α-myosin heavy chain peptide. The mice are treated by continuous per os administration of different doses of ABR-238901 diluted in meglumine buffer, starting at the same time as the injection of virus or of the activated immune cells. Mice with induced myocarditis drinking meglumine buffer alone serve as controls.

### Analyses

Mouse echocardiography, organ collection and flow cytometry are performed as described above, at the same time points in relation to the induction of the disease.

### Results

It is expected that ABR-238901 treatment in the acute phase of myocarditis improves cardiac function, and decreases the intensity of immune and inflammatory cell infiltration in the heart and in heart-draining lymph nodes, in a similar way to the results presented in Examples 12-13 above.

### Conclusions

It is expected that S100A9 blockade with ABR-238901 during the acute phase of the disease inhibits the immune and inflammatory activation, reduces myocardial damage and improves cardiac function in all forms of myocarditis, regardless of the initial trigger of the immune response against the cardiac muscle.

1. A compound of formula (I) 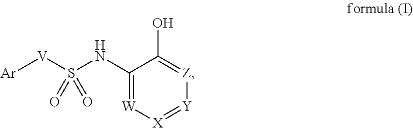
wherein
W is CH or N;
X is N or CR¹;
Y is CR² or N;
Z is CR³ or N;
at least one and at most two of W, X, Y, and Z are N;
R¹ is H, halogen, S(O)₂C1-C3 alkyl, cyano, or C1-C3 alkyl optionally substituted with one or more halogen(s);
R² is H, halogen, cyano, C(O)OH, C(O)OC1-C3 alkyl, C1-C3 alkyl optionally substituted with one or more F; hydroxy-C1-C3 alkyl, S(O)₂C1-C3 alkyl, S(O)₂C3-C6 cycloalkyl or S(O)₂C1-C3 hydroxyalkyl;
R³ is H, halogen or cyano;
V is (CHR⁴)ₘ;
m is 0 or 1;
R⁴ is H or C1-C3 alkyl optionally substituted with one or more halogen(s);
Ar is 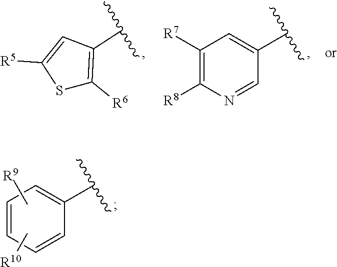
R⁵ is halogen, H, or cyano;
R⁶ is halogen, or H;
R⁷ is halogen, H, C1-C3 alkyl, cyano, S(O)₂C1-C3 alkyl, or phenyl;
R³ is C1-C3 alkoxy optionally substituted with one or more F, C1-C3 alkyl optionally substituted with one or more F, H, halogen, phenoxy, NHR¹¹, or NR¹¹R¹²;
R⁹ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹⁰ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹¹ is C1-C3 alkyl;
R¹² is C1-C3 alkyl; or
R¹¹ and R¹², together with the nitrogen atom to which they are both attached, form a ring of formula 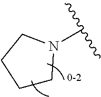
R¹³ is H or C1-C3 alkyl; and
R¹⁴ is H or C1-C3 alkyl;
or a pharmaceutically acceptable salt thereof, for use in the treatment of a disease or disorder involving myocardial inflammation wherein the disease or disorder is selected from the group consisting of myocardial infarction due to myocardial ischemia/reperfusion, myocardial infarction due to myocardial ischemia, myocarditis, sepsis, sepsis-induced cardiomyopathy, and sepsis-induced myocardial inflammation.

2. The compound for use according to claim 1, wherein R⁹ is H or halogen and R¹⁰ is halogen or cyano.

3. The compound for use according to any one of the preceding claims, wherein R⁵ and R⁶ are halogens.

4. The compound for use according to any one of the preceding claims, wherein Ar is

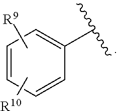

5. The compound for use according to any one of the preceding claims, wherein Ar is

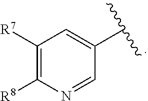

6. The compound for use according to any one of the preceding claims, wherein Ar is

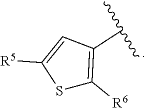

7. The compound or use according to any one of the preceding claims, wherein Y is CR².

8. The compound for use according to any one of the preceding claims, wherein W is N.

9. The compound for use according to any one of the preceding claims, wherein X is N.

10. The compound for use according to any one of the preceding claims, wherein W and X are N.

11. The compound for use according to any one of the preceding claims, wherein Y and Z are both CH.

12. The compound for use according to any one of the preceding claims, wherein Y is CH, Z is CR³, and R³ is halogen.

13. The compound for use according to any one of the preceding claims, wherein Y is CR², Z is CH, and R² is halogen or S(O)₂C1-C3 alkyl.

14. The compound for use according to any one of the preceding claims, wherein Z is N.

15. The compound for use according to any one of the preceding claims, wherein m is 0.

16. The compound for use according to any one of the preceding claims, wherein the compound is selected from

17. The compound for use according to claim 1, wherein
W is N or CH;
X is N or CR¹;
Y is CR²;
Z is N or CR³;
at least one and at most two of W, X, Y, and Z are N;
R¹ is halogen;
R² is H or halogen;
R³ is H or halogen;
V is (CHR⁴)ₘ;
m is 0;
R⁴ is H or C1-C3 alkyl optionally substituted with one or more halogen(s);
Ar is 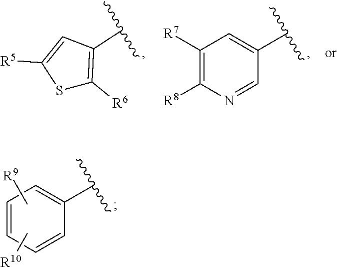
R⁵ is halogen;
R⁶ is halogen;
R⁷ is halogen;
R³ is C1-C3 alkoxy or C1-C3 alkyl;
R⁹ is halogen; and
R¹⁰ is halogen.

18. The compound for use according to any one of the preceding claims, wherein the compound is selected from the group consisting of:
5-bromo-N-(5-chloro-4-hydroxypyridin-3-yl)-6-methoxypyridine-3-sulfonamide;
3,5-dichloro-N-(6-chloro-4-hydroxypyridazin-3-yl)benzene-1-sulfonamide;
2,5-dichloro-N-(4-hydroxypyridin-3-yl)thiophene-3-sulfonamide;
2,5-dichloro-N-(6-chloro-4-hydroxypyridin-3-yl)thiophene-3-sulfonamide;
2,5-dichloro-N-(5-chloro-2-hydroxypyridin-3-yl)thiophene-3-sulfonamide and
5-chloro-N-(5-chloro-4-hydroxypyridin-3-yl)-6-methylpyridine-3-sulfonamide;
or a pharmaceutically acceptable salt thereof.

19. The compound for use according to any one of the preceding claims, wherein the compound is 5-bromo-N-(5-chloro-4-hydroxypyridin-3-yl)-6-methoxypyridine-3-sulfonamide.

20. The compound for use according to any one of the preceding claims, wherein the disease or disorder is myocardial infarction due to myocardial ischemia or myocardial infarction due to myocardial ischemia/reperfusion.

21. The compound for use according to any one of the preceding claims, wherein the myocardial infarction is selected from type 1, type 2, type 4 or type 5 myocardial infarction.

22. The compound for use according to any one of the preceding claims, wherein the myocardial infarction is selected from type 1 or type 2 myocardial infarction.

23. The compound for use according to any one of the preceding claims, wherein the myocardial infarction is type 1 myocardial infarction.

24. The compound for use according to any one of the preceding claims, wherein the myocardial infarction is selected from the group consisting of ST-segment elevation myocardial infarction (STEMI) and non-ST-segment elevation myocardial infarction (NSTEMI).

25. The compound for use according to any one of the preceding claims, wherein the myocardial infarction is ST-segment elevation myocardial infarction (STEMI).

26. The compound for use according to any one of the preceding claims, wherein the myocardial infarction is non-ST-segment elevation myocardial infarction (NSTEMI).

27. The compound for use according to any one of the preceding claims, wherein the disease or disorder is myocarditis.

28. The compound for use according to any one of the preceding claims, wherein the disease or disorder is selected from the group consisting of infectious myocarditis, autoimmune myocarditis and isolated myocarditis.

29. The compound for use according to any one of the preceding claims, wherein the disease or disorder is infectious myocarditis.

30. The compound for use according to any one of the preceding claims, wherein the disease or disorder is autoimmune myocarditis.

31. The compound for use according to any one of the preceding claims, wherein the disease or disorder is isolated myocarditis.

32. The compound for use according to any one of the preceding claims, wherein the disease or disorder is sepsis.

33. The compound for use according to any one of the preceding claims, wherein the disease or disorder is sepsis-induced cardiomyopathy.

34. The compound for use according to any one of the preceding claims, wherein the disease or disorder is sepsis-induced myocardial inflammation.

35. The compound for use according to any one of the preceding claims, wherein the compound is administered in combination with at least one pharmaceutically acceptable excipient.

36. The compound for use according to any one of the preceding claims, wherein the treatment is performed alone or in combination with coronary revascularization.

37. The compound for use according to any one of the preceding claims, wherein the coronary revascularization and/or administration takes place during the acute phase of the disease or disorder.

38. The compound for use according to any one of the preceding claims, wherein the administration is discontinued before onset of the reparatory phase of the disease or disorder.

39. The compound for use according to any one of preceding claims, wherein the disease or disorder is selected from the group consisting of myocardial infarction due to myocardial ischemia/reperfusion and myocardial infarction due to myocardial ischemia, and wherein the administration is discontinued before the onset of the reparatory phase.

40. The compound for use according to any one of the preceding claims, wherein the coronary revascularization and/or administration takes place during the acute phase of the myocardial infarction.

41. The compound for use according to any one of the preceding claims, wherein the administration is discontinued before onset of the reparatory phase of the myocardial infarction.

42. The compound for use according to any one of the preceding claims, wherein the compound is administered at least once daily, such as at least twice daily, e.g. at least three times daily.

43. The compound for use according to any one of the preceding claims, wherein the compound is administered twice daily.

44. The compound for use according to any one of the preceding claims, wherein the compound is administered once daily.

45. The compound for use according to any one of the preceding claims, wherein duration of treatment is one, two, three, four, or five days following onset of the disease or disorder.

46. The compound for use according to any one of the preceding claims, wherein duration of treatment is one, two, three, four, or five days following onset of the myocardial infarction.

47. The compound for use according to any one of the preceding claims, wherein the duration of treatment is one, two, or three days following onset of the myocardial infarction.

48. The compound for use according to any one of the preceding claims, wherein the duration of treatment is three days following onset of the myocardial infarction.

49. The compound for use according to any one of the preceding claims, wherein the duration of treatment is two days following onset of the myocardial infarction.

50. The compound for use according to any one of the preceding claims, wherein the duration of treatment is one day following onset of the myocardial infarction.

51. The compound for use according to any one of the preceding claims, wherein duration of treatment is one, two, three, four, or five days following onset of the sepsis.

52. The compound for use according to any one of the preceding claims, wherein the duration of treatment is five days following onset of the sepsis.

53. The compound for use according to any one of the preceding claims, wherein the duration of treatment is four days following onset of the sepsis.

54. The compound for use according to any one of the preceding claims, wherein the duration of treatment is three days following onset of the sepsis.

55. The compound for use according to any one of the preceding claims, wherein the duration of treatment is two days following onset of the sepsis.

56. The compound for use according to any one of the preceding claims, wherein the duration of treatment is one day following onset of the sepsis.

57. The compound for use according to any one of the preceding claims, wherein the duration of treatment is one week, two weeks, three weeks, or four weeks, following the onset of the disease or the disorder.

58. The compound for use according to any one of the preceding claims, wherein the duration of treatment is one week, two weeks, three weeks, or four weeks following the onset of the myocarditis.

59. The compound for use according to any one of the preceding claims, wherein the duration of treatment is four weeks following onset of the myocarditis.

60. The compound for use according to any one of the preceding claims, wherein the duration of treatment is three weeks following onset of the myocarditis.

61. The compound for use according to any one of the preceding claims, wherein the duration of treatment is two weeks following onset of the myocarditis.

62. The compound for use according to any one of the preceding claims, wherein the duration of treatment is one week following onset of the myocarditis.

63. The compound for use according to any one of the preceding claims, wherein the route of administration is oral, intravenous, subcutaneous and/or intramuscular.

64. A method of treating a disease or disorder involving myocardial inflammation in a subject in need thereof, the method comprising the steps of:
a. providing a blood sample obtained from said subject;
b. measuring the level of S100A8/A9 in the blood sample;
c. comparing the level of S100A8/A9 in the blood of said subject to that of a healthy subject and/or a standard;
d. determining if the level of S100A8/A9 in said blood sample is higher compared to that of the healthy subject and/or the standard;
e. administering the compound according to any one of the preceding claims to the subject having a higher S100A8/A9 level than that of the healthy subject and/or the standard.

65. A method of treating a disease or disorder involving a myocardial inflammation, said method comprising administering a therapeutically effective amount of a compound according to formula (I), 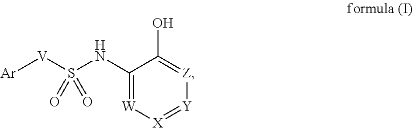
wherein
W is CH or N;
X is N or CR¹;
Y is CR² or N;
Z is CR³ or N;
at least one and at most two of W, X, Y, and Z are N;
R¹ is H, halogen, S(O)₂C1-C3 alkyl, cyano, or C1-C3 alkyl optionally substituted with one or more halogen(s);
R² is H, halogen, cyano, C(O)OH, C(O)OC1-C3 alkyl, C1-C3 alkyl optionally substituted with one or more F; hydroxy-C1-C3 alkyl, S(O)₂C1-C3 alkyl, S(O)₂C3-C6 cycloalkyl or S(O)₂C1-C3 hydroxyalkyl;
R³ is H, halogen or cyano;
V is (CHR⁴)ₘ;
m is 0 or 1;
R⁴ is H or C1-C3 alkyl optionally substituted with one or more halogen(s);
Ar is 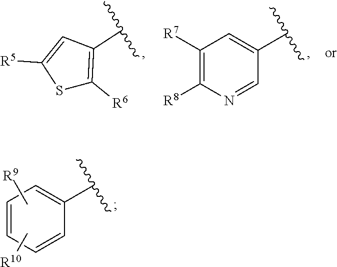
R⁵ is halogen, H, or cyano;
R⁶ is halogen, or H;
R⁷ is halogen, H, C1-C3 alkyl, cyano, S(O)₂C1-C3 alkyl, or phenyl;
R³ is C1-C3 alkoxy optionally substituted with one or more F, C1-C3 alkyl optionally substituted with one or more F, H, halogen, phenoxy, NHR¹¹, or NR¹¹R¹²;
R⁹ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹⁰ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹¹ is C1-C3 alkyl;
R¹² is C1-C3 alkyl; or
R¹¹ and R¹², together with the nitrogen atom to which they are both attached, form a ring of formula 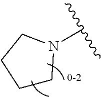
R¹³ is H or C1-C3 alkyl; and
R¹⁴ is H or C1-C3 alkyl;
or a pharmaceutically acceptable salt thereof to a subject in need thereof.

66. Use of a compound according to formula (I) 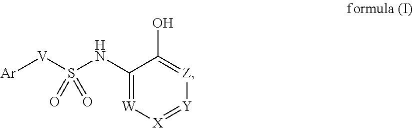
wherein
W is CH or N;
X is N or CR¹;
Y is CR² or N;
Z is CR³ or N;
at least one and at most two of W, X, Y, and Z are N;
R¹ is H, halogen, S(O)₂C1-C3 alkyl, cyano, or C1-C3 alkyl optionally substituted with one or more halogen(s);
R² is H, halogen, cyano, C(O)OH, C(O)OC1-C3 alkyl, C1-C3 alkyl optionally substituted with one or more F; hydroxy-C1-C3 alkyl, S(O)₂C1-C3 alkyl, S(O)₂C3-C6 cycloalkyl or S(O)₂C1-C3 hydroxyalkyl;
R³ is H, halogen or cyano;
V is (CHR⁴)ₘ;
m is 0 or 1;
R⁴ is H or C1-C3 alkyl optionally substituted with one or more halogen(s);
Ar is 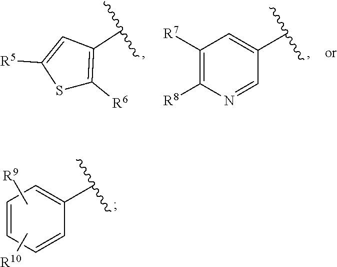
R⁵ is halogen, H, or cyano;
R⁶ is halogen, or H;
R⁷ is halogen, H, C1-C3 alkyl, cyano, S(O)₂C1-C3 alkyl, or phenyl;
R³ is C1-C3 alkoxy optionally substituted with one or more F, C1-C3 alkyl optionally substituted with one or more F, H, halogen, phenoxy, NHR¹¹, or NR¹¹R¹²;
R⁹ is H, halogen, cyano, C1-C3 alkyl optionally substituted with one or more F; C1-C3 alkylthio optionally substituted with one or more F; C1-C3 alkoxy optionally substituted with one or more F; or C(O)NR¹³R¹⁴;
R¹⁰ is H, halogen, cyano, C1-C3 alkyl optionally substituted with one or more F C1-C3 alkylthio optionally substituted with one or more F; C1-C3 alkoxy optionally substituted with one or more F; or C(O)NR¹³R¹⁴;
R¹¹ is C1-C3 alkyl;
R¹² is C1-C3 alkyl; or
R¹¹ and R¹², together with the nitrogen atom to which they are both attached, form a ring of formula 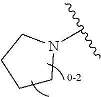
R¹³ is H or C1-C3 alkyl; and
R¹⁴ is H or C1-C3 alkyl;
or a pharmaceutically acceptable salt thereof for the manufacture of a medicament for the treatment of a disease or disorder involving a myocardial inflammation.
